(19) United States
(12) Patent Application Publication
(10) Pub. No.: US 2004/0122001 A1
(43) Pub. Date: Jun. 24, 2004

(54) Pharmaceutical compounds

(76) Inventors: Javier Agejas-Chicharro, Alcobendas (ES); Ana Belen Bueno Melendo, Alcobendas (ES); Nicholas Paul Camp, Windlesham (GB); Jeremy Gilmore, Windlesham (GB); Alma Maria Jimenez-Aguado, Alcobendas (ES); Carlos Lamas-Peteira, Alcobendas (ES); Alicia Marcos-Llorente, Alcobendas (ES); Michael Philip Mazanetz, Windlesham (GB); Carlos Montero Salgado, Alcobendas (ES); Graham Henry Timms, Windlesham (GB); Andrew Caerwyn Williams, Windlesham (GB)

Correspondence Address: Michael Sayles, Eli Lilly & Company, Patent Division, PO Box 6288, Indianapolis, IN, 46206-6288, US

(21) Appl. No.: 10/433,912

(22) PCT Filed: Dec. 19, 2001
(86) PCT No.: PCT/US01/45856

(30) Foreign Application Priority Data
Dec. 20, 2000 (GB) 0031084.7

(51) Int. Cl. A61K 31/551; A61K 31/496; C07D 413/14

(52) U.S. Cl. 514/218; 514/253.09; 514/254.02; 514/254.05; 514/254.04; 540/575; 544/360; 544/368; 544/369; 514/254.03

(57) ABSTRACT

This invention relates to compounds of formula (I) where R¹ to R¹², —W—V—, —X—Y—, m and n have the values defined in claim 1, their preparation and use as pharmaceuticals.

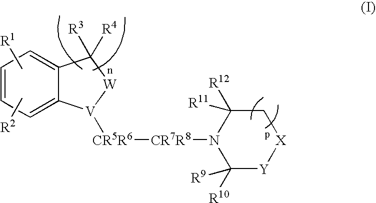

This invention relates to novel compounds, their preparation and use as pharmaceuticals.

Certain isochroman compounds useful as antipsychotics and in the treatment of disorders of the central nervous system, are disclosed in WO 95/18118 and WO 97/02259.

The compounds of the invention are of the following general formula:

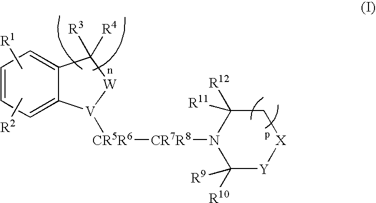

in which

R¹ is

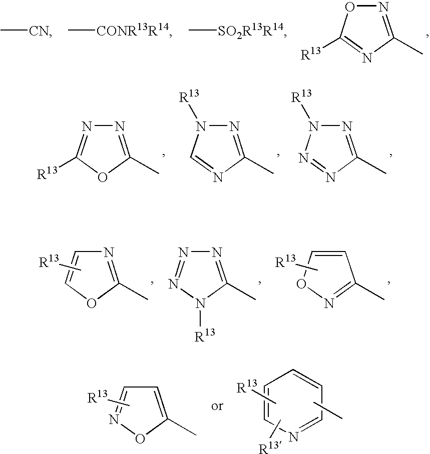

where R¹³ and R¹⁴ are each hydrogen or C₁₋₆ alkyl, or R¹³ and R¹⁴ taken together with the nitrogen atom to which they are attached form a morpholino, pyrrolidino or piperidinyl ring optionally substituted with one or two C₁₋₆ alkyl groups;

R¹³′ is selected from hydrogen, C₁₋₆ alkyl, C₁₋₆ alkoxy, carboxy, hydroxy, cyano, halo, trifluoromethyl, nitro, amino, C₁₋₆ acylamino, C₁₋₆ alkylthio, phenyl or phenoxy;

R² is one of the values defined for R¹, or hydrogen, C₁₋₆ alkyl, C₁₋₆ alkoxy or halo;

R³, R⁴, R⁵, R⁶, R⁷, R⁸, R¹⁰ and R¹² are each hydrogen or C₁₋₆ alkyl;

R⁹ and R¹¹ are each hydrogen, C₁₋₆ alkyl or —(CH₂)_{q}—OR²⁰,

wherein R²⁰ is C₁₋₆ alkyl;

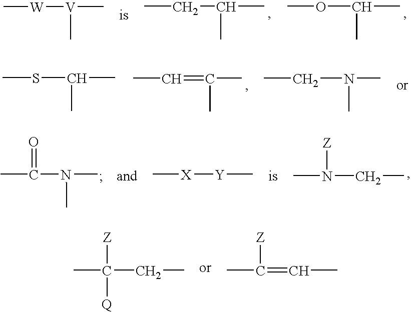

where Z is

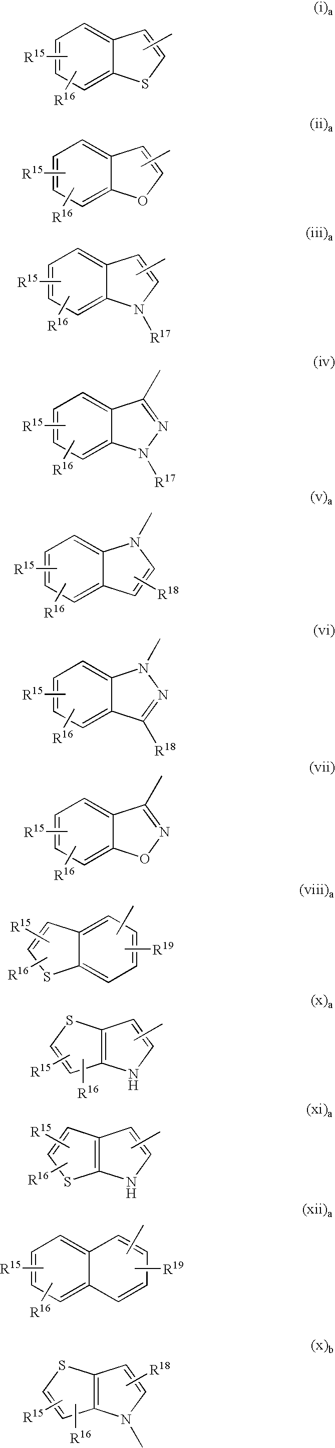

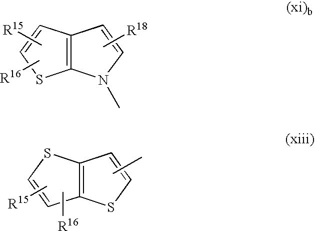

where R¹⁵, R¹⁶ and R¹⁹ are each hydrogen, halo, C₁₋₆ alkyl or C₁₋₆ alkoxy, carboxy-C₁₋₆ alkyl, cyano, halogen, trifluoromethyl, trifluoromnethoxy, nitro, amino, C₁-C₆ acylamino and C₁-C₆ alkylthio; and R¹⁷ and R¹⁸ are each hydrogen or C₁₋₆ alkyl;

Q is hydrogen, halo, nitrile, carboxy-C₁_₆ alkyl, hydroxy, C₁₋₆ alkyl or C₁₋₆ alkoxy;

and pharmaceutically acceptable salts thereof.

The compounds of the invention and their pharmaceutically acceptable salts are indicated for use in the treatment of disorders of the central nervous system.

In the above formula (I), a C₁₋₆ alkyl group can be branched or unbranched and, for example, includes methyl, ethyl, propyl, isopropyl, butyl, isobutyl, t-butyl, pentyl and hexyl, and is preferably methyl or ethyl, and especially methyl. A C₁₋₆ alkoxy group is one such alkyl group linked to a ring through an oxygen atom, and is preferably methoxy or ethoxy, and especially methoxy. A halo group is preferably fluoro, chloro or bromo, and especially fluoro. A (C₁-C₆)alkylthio is an alkyl group linked to a sulphur atom, where the alkyl is as defined above. A (C₁-C₆)alkylthio group includes for example thiomethyl or thioethyl. A C₁-C₆ acylamino group is an alkyl group linked to an amide group, where the alkyl is as defined above, and is preferably of the formula R^{IV}—NH—CO— where R^{IV} is C₁-C₅ alkyl. A C₁-C₆ acylamino group includes for example acetamide.

When n is 2 it will be appreciated that the values of R³ and R⁴ in the repeated-units can be different.

Preferably R² is hydrogen, C₁₋₆ alkyl, C₁₋₆ alkoxy or halo.

Preferred compounds of the invention have one or more of the following features:

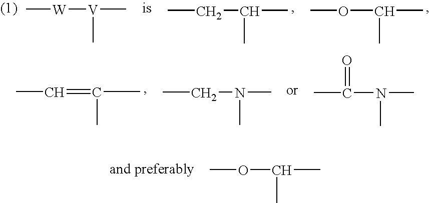

(2) R³, R⁴, R⁵, R⁶, R⁷, R⁸, R⁹, R¹⁰, R¹¹ and R¹² are each hydrogen or C₁₋₆ alkyl;

(3) groups R³ to R¹² are hydrogen, or R³ to R¹⁰ and R¹² are hydrogen and R¹¹ is C₁₋₆ alkyl, especially methyl

(4) R¹¹ is methyl, ethyl or propyl

(5) R¹¹ is C₁₋₆ alkyl or —(CH₂)_{q}—OR²⁰, and R²⁰ is C₁₋₆ alkyl

(6) q is 1

(7) R¹ is —CONR¹³R¹⁴, and R¹³ and R¹⁴ are hydrogen

(8) R¹ is —CONR¹³R¹⁴, and R¹³ and R¹⁴ are each hydrogen or methyl

(9) R² is hydrogen

(10) R² is hydrogen, chloro, fluoro or methyl

(11) R¹⁵, R¹⁶ and R¹⁸ are each hydrogen, halo or methoxy

(12) R¹⁵, R¹⁶ and R¹⁸ are each hydrogen, halo, cyano or methoxy

(13) R¹⁷ is hydrogen or C₁₋₆ alkyl, preferably methyl

(14) R¹⁹ is hydrogen, halo, cyano or C₁₋₆ alkyl

(15) p is 0 or 1

(16) p is 1

(17) Z is

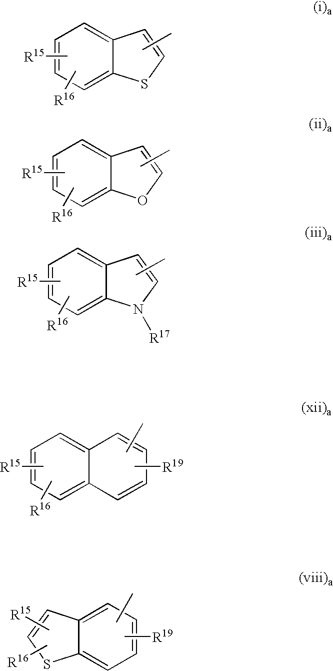

(18) Z is

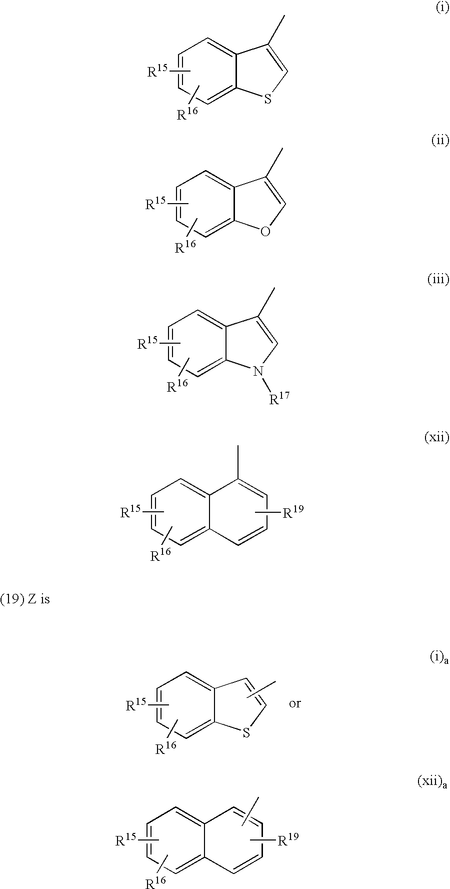

In one embodiment the compounds of the present invention are of the above general formula (I), in which R¹ is

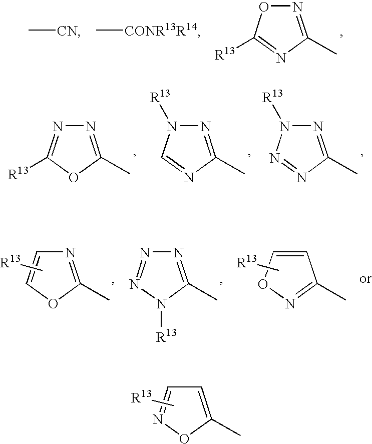

where R¹³ and R¹⁴ are each hydrogen or C₁₋₆ alkyl, or R¹³ and R¹⁴ taken together with the nitrogen atom to which they are attached form a morpholino, pyrrolidino or piperidinyl ring optionally substituted with one or two C₁₋₆ alkyl groups;

R² is one of the values defined for R¹, or hydrogen, C₁₋₆ alkyl, C₁₋₆ alkoxy or halo;

R³, R⁴, R⁵, R⁶, R⁷, R⁸, R⁹, R¹⁰, R¹¹ and R¹² are each hydrogen or C₁₋₆ alkyl;

n is 1 or 2;

p is 0, 1 or 2;

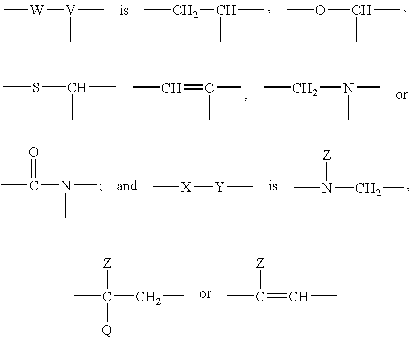

where Z is

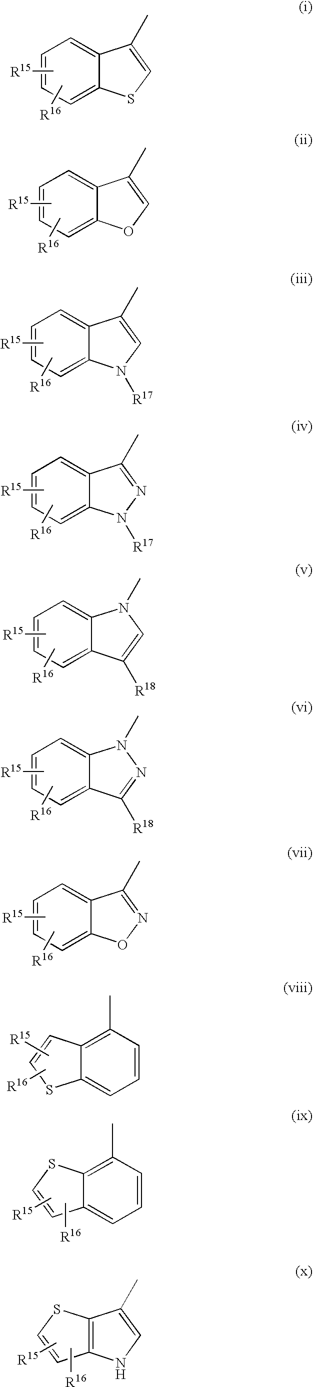

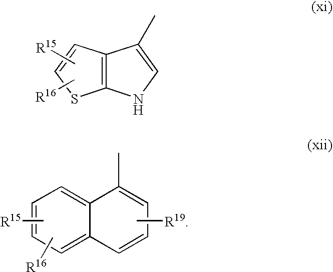

where R¹⁵, R¹⁶ and R¹⁹ are each hydrogen, halo, C₁₋₆ alkyl or C₁₋₆ alkoxy, carboxy-C₁₋₆ alkyl, cyano, halogen, trifluoromethyl, trifluoromethoxy, nitro, amino, C₁-C₆ acylamino and C₁-C₆ alkylthio; and R¹⁷ and R¹⁸ are hydrogen or C₁₋₆ alkyl;

Q is hydrogen, halo, nitrile, carboxy-C1-6 alkyl, hydroxy, C₁₋₆ alkyl or C₁₋₆ alkoxy;

and pharmaceutically acceptable salts thereof, in which preferred compounds have one or more of the following features:

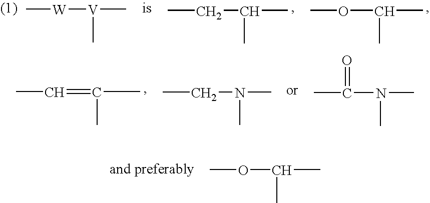

(2) groups R³ to R¹² are hydrogen, or R³ to R¹⁰ and R¹² are hydrogen and R¹¹ is C₁₋₆ alkyl, especially methyl

(3) R¹ is —CONR¹³R¹⁴, and R¹³ and R¹⁴ are hydrogen

(4) R² is hydrogen

(5) p is 0 or 1

(6) Z is

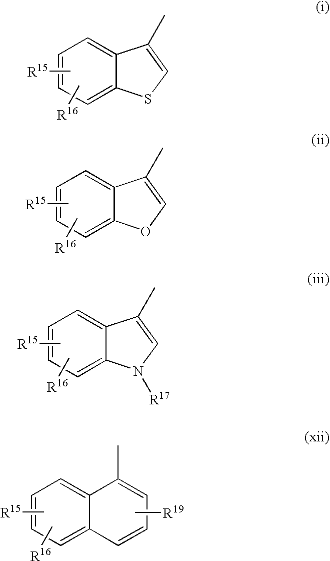

(7) R¹⁵, R¹⁶ and R¹⁸ are each hydrogen, halo or methoxy

(8) R¹⁷ is hydrogen or C₁₋₆ alkyl, preferably methyl.

A preferred group of compounds is of the formula:

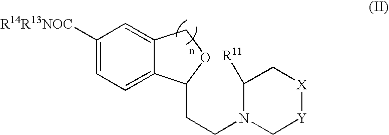

in which n is 1 or 2, R¹³ and R¹⁴ are each hydrogen or C₁₋₆ alkyl, and are preferably both hydrogen, R¹¹ is hydrogen or C₁₋₆ alkyl, preferably methyl, and —X—Y— is

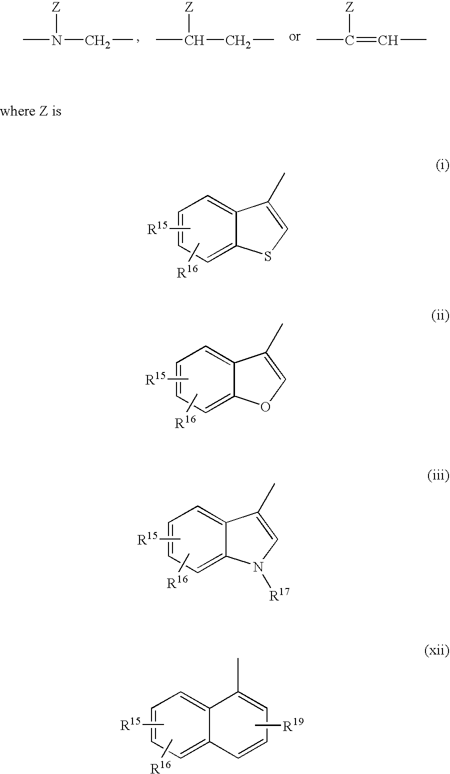

and R¹⁵, R¹⁶ and R¹⁹ are each hydrogen, halo or alkoxy, and R¹⁷ is hydrogen or C₁₋₆ alkyl; and pharmaceutically acceptable salts thereof.

As indicated above, it is, of course, possible to prepare salts of the compounds of the invention and such salts are included in the invention. Acid addition salts are preferably the pharmaceutically acceptable, non-toxic addition salts with suitable acids, such as those with inorganic acids, for example hydrochloric, hydrobromic, nitric, sulphuric or phosphoric acids, or with organic acids, such as organic carboxylic acids, for example glycollic, maleic, hydroxymaleic, fumaric, malic, tartaric, citric, salicyclic, o-acetoxybenzoic, or organic sulphonic, 2-hydroxyethane sulphonic, toluene-p-sulphonic, naphthalene-2-sulphonic or bisethane sulphonric acids. The phosphate is a most preferred salt.

In addition to the pharmaceutically acceptable salts, other salts are included in the invention. They may serve as intermediates in the purification of compounds or in the preparation of compounds or in the preparation of other, for example pharmaceutically acceptable acid addition salts, or are useful for identification, characterisation or purification.

It will be appreciated that the compounds of the invention can contain one or more asymmetric carbon atoms which gives rise to isomers. The compounds are normally prepared as racemic mixtures, but individual isomers can be isolated by conventional techniques if so desired. Such racemic mixtures and individual optical isomers form part of the present invention, the compounds being employed as racemates or in enantiomerically pure form.

Preferred compounds of the invention are those of formula:

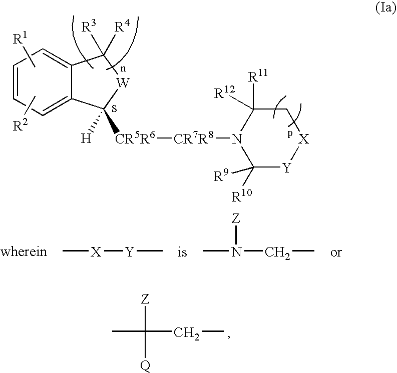

R¹ to R¹², Q, Z, n and p have the values defined for formula I above, —W— is —CH₂—, —O—, or —S—.

Compounds of formula Ia can contain more asymmetric carbons. For example when the R¹¹ and R¹² groups are different, this gives rise to isomers R and S, such as compounds of formula (Ib) and (Ic). Said isomers are also an aspect of the invention.

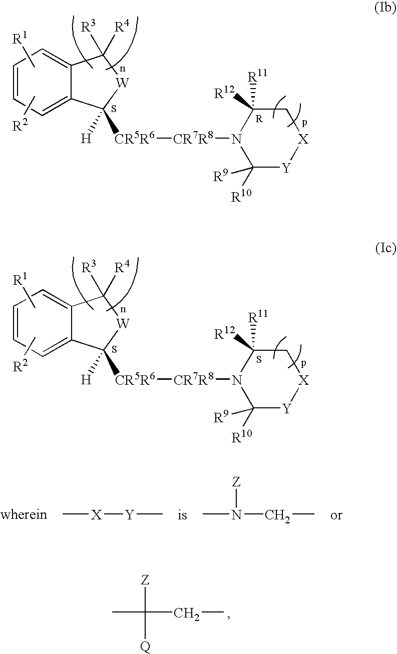

R¹ to R¹², Q, Z, n and p have the values defined for formula I above, preferably R¹¹ is C₁₋₆ alkyl, especially methyl and R¹² is H, —W— is —CH₂—, —O—, or —S—.

Preferred compounds of the invention are those compounds of the formula Ib.

In the same way, when the R⁹ and R¹⁰ groups are different in compounds of formula Ia, this also gives rise to isomers R and S, such as compounds of formula (Id) and (Ie). Said isomers are also an aspect of the invention.

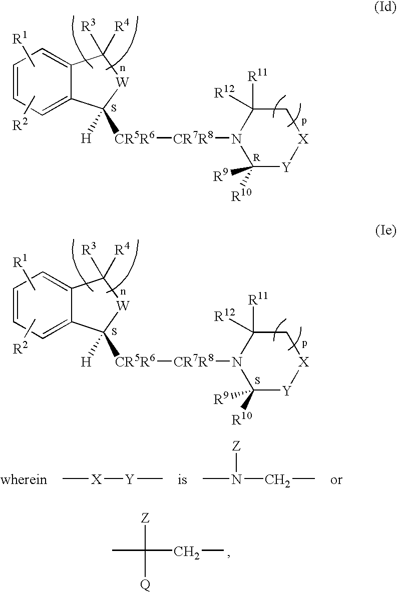

R¹ to R¹², Q, Z, n and p have the values defined for formula I above, preferably R¹¹ is C₁₋₆ alkyl, especially methyl and R¹² is H, —W— is —CH₂—, —O—, or —S—.

Preferred compounds of the invention are those compounds of the formula Id.

The compounds of the invention can be produced by reacting a compound having the formula:

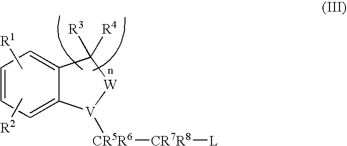

where L is a leaving group, with a compound of the formula:

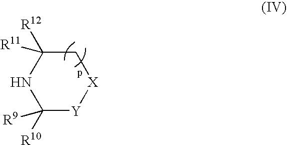

where the substituents have the values defined for formula (I) above.

The reaction is preferably carried out in the presence of a base such as potassium carbonate, in an organic solvent such as a polar aprotic solvent, for example, acetonitrile, at a temperature of from 20° C. to 100° C. Examples of suitable leaving groups are mesylate, tosylate, triflate, chloride, bromide and iodide.

Intermediate compounds of formula (III) can, for example, be prepared from the corresponding alcohols of the formula:

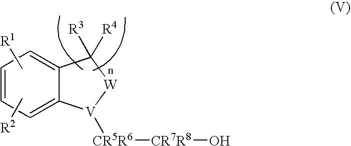

using standard methods known in the literature such as the ones shown in March, Advanced Organic Chemistry, Fourth Edition, for example the methods mentioned on pages 353 and 354.

Compounds of formula (IV) can be prepared by a variety of methods well known in the art. Substituted 3-(1,2,3,6-tetrahydro-4-pyridinyl)-1H-indoles, fluoro substituted -3-(4-piperidinyl)-1H-indoles and (3R)-6-fluoro-3-(3-pyrrolidinyl)-1H-indole were prepared using methods described in European patent application 1999 EP 897921 and WO patents 9958525 and 002341. Substituted and unsubstituted 4-(1-naphthyl)-1,2,3,6-tetrahydropyridines and 4-(1-naphthyl)piperidines were prepared using methods described in U.S. Pat. Nos. 5,472,966, 5,250,544, and 5,292,711. Substituted and unsubstituted 1-(1-naphthyl)piperazines were prepared using methods described in U.S. Pat. No. 5,166,156. (2R,4S)-2-methyl-4-(2-naphthyl)piperidine was prepared using methods referred to in Med. Chem. Res. (1997), 7(4), 207-218. Substituted and unsubstituted 4-(1-benzopyran-3-yl)-1,2,3,6-tetrahydropyridines and 4-(1-benzopyran-3-yl)piperidines were prepared using methods described in Eur. Pat. Appl. (1992) EP 466585 or in Japanese patent JP 2000086603. 6-fluoro-3-(1,2,3,6-tetrahydro-4-pyridinyl)-1,2-benzisoxazole was prepared methods based on U.S. Pat. No. 3,678,062. Substituted and unsubstituted 6-fluoro-1-3-(1,2,3,6-tetrahydro-4-pyridinyl)-1H-indazoles were prepared by methods described in EP 135781 (1985). 4-(Thieno[3,2-b]pyrrol-6-yl)-1,2,3,6-tetrahydropyridine was prepared by methods found in Heterocycl. Commun. (1999), 5(4) 305-310. Substituted and unsubstituted 4-(1-benzothieny-7-yl)-1,2,3,6-tetrahydropyridines and 4-(4-fluoro-1-benzopyran-7-yl)-1,2,3,6-tetrahydro-pyridine were prepared using methods described in WO-0000198. 6-Substituted 2-[3,4-dihydro-1H-2-benzopyran-1-yl]ethyl methanesulfonates were made by procedures described in WO 9518118. 5-Methoxy-3-(1,2,3,6-tetrahydro-4-pyridinyl)-1H-indole could be obtained from Tocris Cookson. 3-[2-(4-piperidinyl)ethyl)-1H-indoles were prepared using methods described in J. Med. Chem. 1993, 36(15), 2242 and J. Med. Chem, 36(9) 1194.

Compounds of formula (IV) wherein R¹⁰ is —CH₂—OR²⁰ can be prepared as described in the synthetic scheme below:

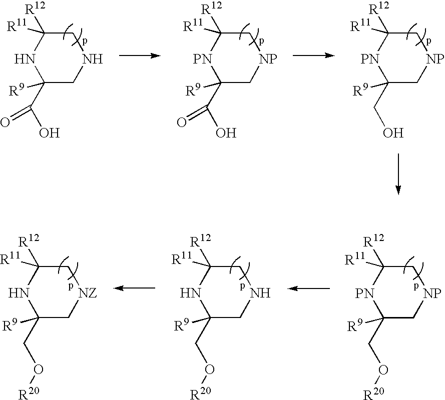

wherein the nitrogen atoms are protected with a suitable protecting group such as N-tert-butoxycarbonyl (BOC) using methods described in Greene and Wuts, Protecting Groups in Organic Synthesis, 3rd. Ed., John Wiley & Sons, followed by reduction of the acid moiety to the alcohol, alkylation of said alcohol and deprotection of the nitrogen atoms.

The unprotected piperidine is then reacted with a compound of formula Z-Lⁱⁱⁱ in the presence of a palladium catalyst such as palladium acetate, BINAP ((R)-2,2′-bis(diphenylphosphino)-1,1′-binaphthyl) and a base such as Cesium carbonate.

The nitrogen groups can for example be protected with a BOC group using di-tert-butyl dicarbonate in the presence of a base such as sodium hydroxide in an organic solvent such as ethanol.

The reduction is preferably carried out in the presence of a reducing agent such as borane dimethyl sulfide in a organic solvent such as THF at a temperature ranging from 0° C. to room temperature.

The alkylation reaction is preferably carried out in an organic solvent such as DMF, in the presence of a base such as sodium hydride and an alkylating agent such as iodomethane (for compounds where R²⁰ is methyl).

Compounds of formula (V) wherein R⁷ and R⁸ are hydrogen can, for example, be prepared from the appropriate esters of the formula:

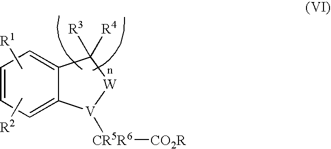

where R is C₁₋₆ alkyl. Such esters can be reduced in the presence of a reducing agent such as lithium borohydride or lithium aluminium hydride in a suitable organic solvent such as tetrahydrofuran (THP).

Compounds of formula (V) wherein R¹ is —CONR¹³R¹⁴ can be prepared from the appropriate halo-substituted alcohols of the formula:

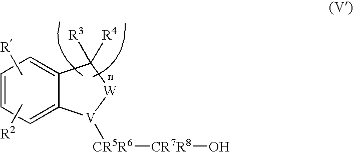

where R′ is a halo group, such as chloro, bromo or iodo. Such alcohols are prepared using the same conditions as shown above. Then the alcohol is protected using a suitable protecting group as shown in Greene and Wuts, Protecting Groups in Organic Synthesis, 3rd. Ed., John Wiley & Sons. Preferred protecting groups are silyloxy protecting groups such as for example tertbutyldimethylsilyl group.

The halogen is then converted to the corresponding carboxamido group (—CONR¹³R¹⁴), via formation of the corresponding carboxy group and then condensation with the appropriate amine of formula HNR¹³R¹⁴. The carboxy group is formed by reaction of the intermediate organolithium reagent with carbon dioxide in a suitable organic solvent such as THF. The subsequent condensation reaction with the appropriate amine of formula HNR¹³R¹⁴ is preferably carried out in the presence of a coupling reagent such as carbonyldiimidazole (CDI) in a suitable solvent such as dioxan.

Similarly the halogen can be converted in one step to the corresponding carboxamido group by reaction of the organolithium reagent described above with trimethylsilyl isocyanate.

Alternatively the halogen can be converted to the corresponding carboxamido group by reaction with an inorganic cyanide, such as zinc cyanide, in the presence of a palladium catalyst, such as tris(dibenzylideneacetone) dipalladium, and a phosphine ligand, such as tri-tert-butylphosphine. The reaction is carried out in a suitable solvent such as dioxan, usually at reflux.

Then the alcohols are deprotected using standard methods known in the literature, such as Greene and Wuts, Protecting Groups in Organic Synthesis, 3rd. Ed., John Wiley & Sons.

Compounds of the formula (VI) wherein

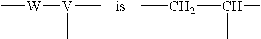

and R⁵, R⁶, R⁷ and R⁸ are hydrogen can be prepared from the appropriate ketones of formula (VII) as shown in Scheme I below.

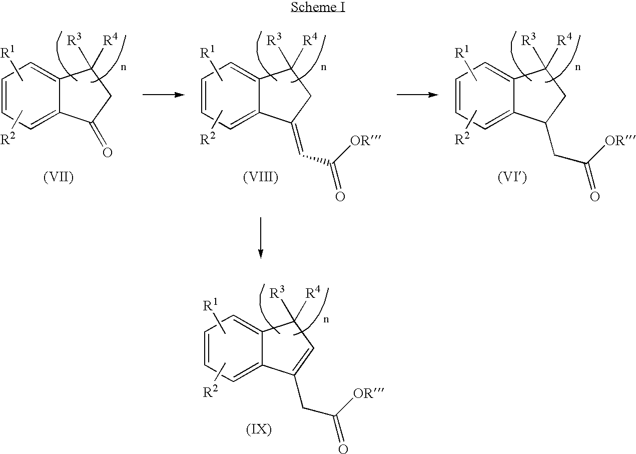

Such ketones react with activated ylides such as for example a phosphonate of the formula (R″O)₂P(O)CH₂CO₂R′″, wherein R″ and R′″ are each C₁₋₆ alkyl, in the presence of a base such as sodium hydride in a suitable solvent such as for example THF to form the corresponding unsaturated ester (VIII). The alkene is reduced for example via hydrogenation in the presence of a catalyst such as Pd on charcoal in a suitable solvent such as ethanol or methanol.

Unsaturated esters of formula (IX) can be prepared via isomerisation of the corresponding unsaturated ester of formula (VIII) as shown in scheme I above. This reaction is carried out in the presence of a suitable base such as sodium methanide in a suitable solvent such as THF.

Compounds of the formula (VI) wherein

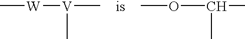

can be prepared as shown in scheme II from the appropriate lactones of formula (X).

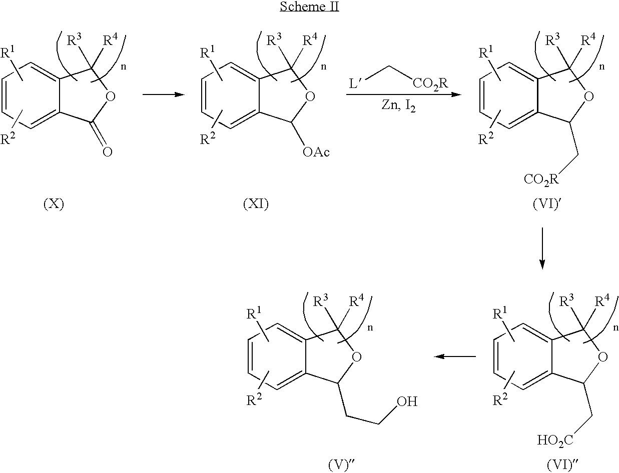

Such lactones are converted to the corresponding hemiacetals via reduction of the lactone using a reducing agent such as diisobutylaluminium hydride (DIBAL) in the presence of a suitable solvent such as dichloromethane, followed by the protection of the intermediate hemiacetal with a suitable protecting group such as acetate. The protected hemiacetal is reacted with an appropriate organozincate derived from the corresponding haloacetal of formula L′-CH₂—CO₂R wherein L′ is a halogen group such as bromo or iodo and R has the value defined above, in the presence of a Lewis acid such as trimethylsilyltriflate to form esters of the formula (VI)′.

Alternatively the hemiacetal is reacted directly with an activated ylid such as for example a phosphonate of the formula (R″O)₂P(O)CH₂CO₂R′″, wherein R″ and R′″ are each C₁₋₆ alkyl, in the presence of a base such as cesium carbonate in a suitable solvent such as for example THF, to form the corresponding ester (VI)′.

Such esters can be converted to the corresponding alcohols using the method mentioned above. Alternatively they can be hydrolysed in acidic conditions to the acid, followed by formation of the mixed anhydride and final reduction of such a mixed anhydride to the corresponding alcohol of formula (V)″.

Alternatively compounds above wherein n is 2 can be synthesised via standard acid catalysed cyclisation of the corresponding phenyl alcohol of formula (XII) with an appropriate aldehyde of the formula CHO—CH₂—COOR^{iv} or its corresponding acetal of the formula (R^{v}O)₂CH—CH₂—COOR^{iv}, wherein R^{iv} and R^{v} are each independently a C₁-C₆ alkyl group, in the presence of a Lewis acid such as titanium tetrachloride in a suitable solvent such as dichloromethane, see Scheme III below.

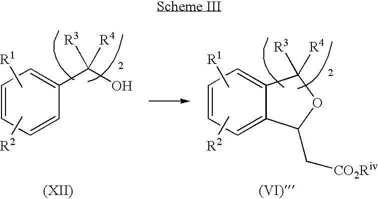

Compounds of the formula (V) wherein

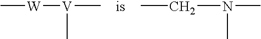

can be prepared as shown in scheme IV from the appropriate quinolines of formula (XIII).

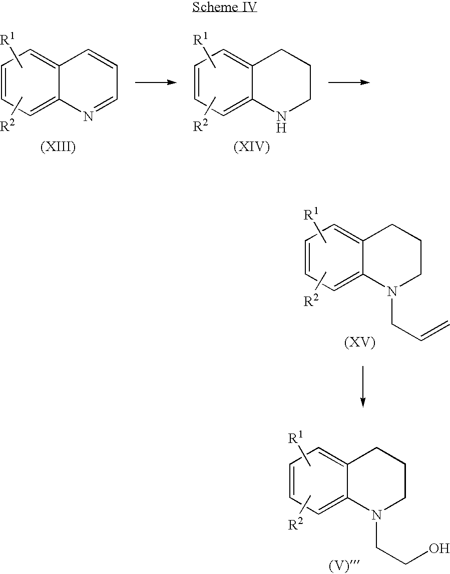

Such quinolines are converted to the corresponding 1,2,3,4 tetrahydroquinolines by reduction, for example by hydrogenation in the presence of ammonium formate and a suitable catalyst such as Palladium on charcoal in a suitable solvent such as methanol. The tetrahydroquinoline is then alkylated with allyl halide for example allyl bromide in the presence of a suitable base such as sodium hydride in a suitable solvent such as dimethylformamide (DMF). The double bond of the allyl group is then cleaved for example via ozonolysis and subsequently the aldehyde formed is reduced with a suitable reducing agent such as sodium borohydride to give the corresponding alcohol. Alternatively such a double bond can be cleaved for example with osmium tetroxide and sodium periodate in the presence of a suitable reducing agent such as sodium borohydride. This way the aldehyde formed is reduced in situ.

Compounds of the formula (V) wherein

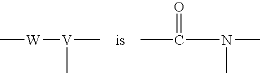

can be prepared as shown in scheme V from the appropriate 2-oxo-1,2,3,4-tetrahydroquinoline of formula (XVI).

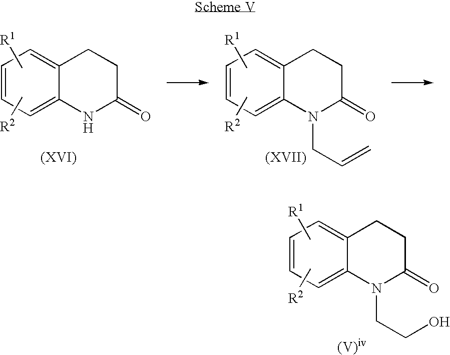

Such 2-oxo-1,2,3,4-tetrahydroquinolines can be alkylated with an allyl halide for example allyl bromide in the presence of a suitable base such as sodium hydride in a suitable solvent such as dimethylformamide (DMF). The allyl group can be converted to the corresponding alcohol using the method shown above.

Compounds of the invention can also be synthesised via reaction of the corresponding amine of the formula (XIX) with a compound of the formula Z-Lⁱⁱⁱ wherein Lⁱⁱⁱ is a leaving group such as triflate or a halide such as bromide or iodide.

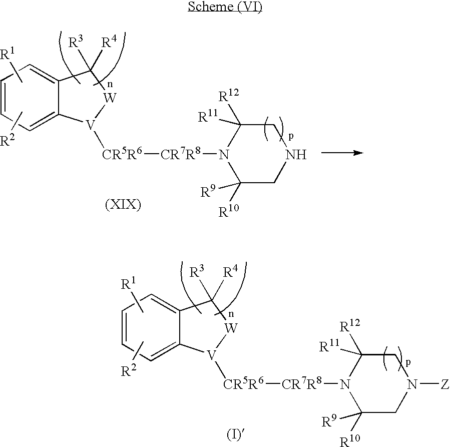

Such reactions are usually carried out in the presence of a palladium catalyst such as palladium acetate and a base such as potassium tertbutoxide.

Some intermediates of the general formula Z-Lⁱⁱⁱ wherein Lⁱⁱⁱ is a halogen group such as bromo are commercially available. Alternatively, they can be synthesised from known literature routes, such as by brominating the corresponding aromatic group with NBS. Intermediates wherein Lⁱⁱⁱ is a triflate can be prepared using methods known in the art such as from the corresponding ketones in the presence of triflic anhydride. Such intermediates are illustrated in scheme (VIII) for compound wherein Z is (i) and (xii), but it will appreciated that such method can be used for any values of Z.

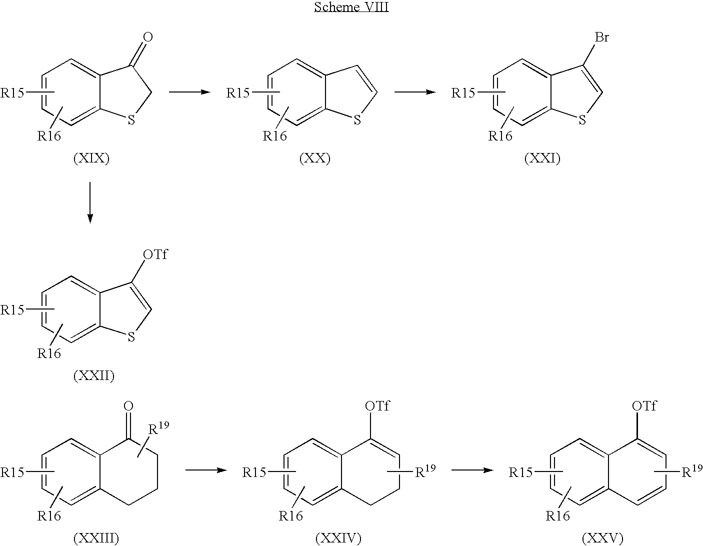

Compounds of formula (I) wherein R¹ is

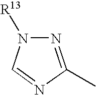

can be synthesised from the corresponding amide intermediates of formula (V) wherein the alcohol moiety is protected with an appropriate alcohol protecting group P, such as those shown in Greene and Wuts, Protecting Groups in Organic Synthesis, 3rd. Ed., John Wiley & Sons.

Such intermediates are cyclised via reaction with dimethylformamide dimethylacetal in a suitable solvent such as toluene, followed by reaction with the corresponding hydrazine of the formula R13-NH—NH2 in a suitable solvent such as for example methanol. Then the alcohols are deprotected using methods known in the art such as those shown in Greene and Wuts, Protecting Groups in Organic Synthesis, 3rd. Ed., John Wiley & Sons.

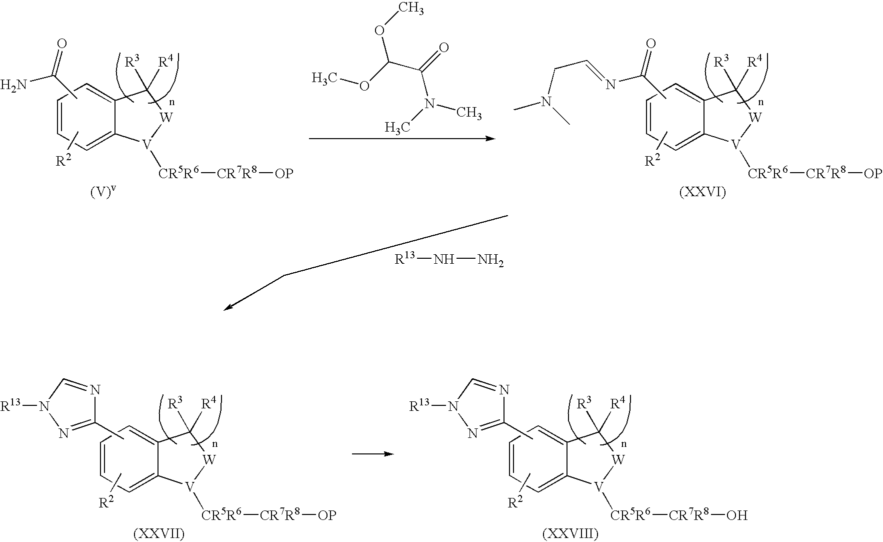

As described above the compounds of the invention can have an asymmetric centre, said compounds, for example compounds of formula Ia, can be prepared in a similar way as those compounds of general formula I, by reacting a compound of formula:

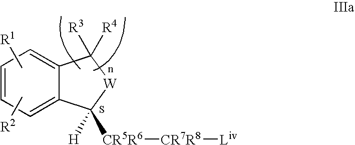

where n and R¹ to R⁸ have the values defined for formula I above, —W— is —CH₂—, —O—, or —S—, and L^{iv} is a leaving group, with a compound of formula (IV).

The reaction is preferably carried out using the same conditions as described above, such as in the presence of a base such as potassium carbonate, in an organic solvent such as a polar aprotic solvent, for example, acetonitrile, at a temperature of from 20° C. to 100° C. Examples of suitable leaving groups are mesylate, tosylate, triflate, chloride, bromide and iodide.

Intermediate compounds of formula (IIIa) can, for example, be prepared from the corresponding alcohols of the formula:

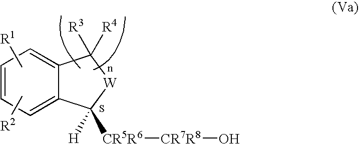

where the substituents have the values defined for formula (IIIa) above, using standard methods known in the literature such as the ones shown in March, Advanced Organic Chemistry, Fourth Edition, for example the methods mentioned on pages 353 and 354.

Said alcohols of formula (Va) can be prepared via methods known in the literature such as for example the procedure described in TenBrink et al., J. Med. Chem., 1996, 39, 2435-2437.

In the same way compounds of the invention having two asymmetric carbon atoms such as compounds of formula (Ib), can be prepared by reacting the corresponding chiral intermediates such as a compound of formula (IIIa) with a compound of the formula:

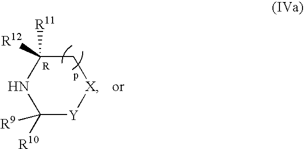

for compounds of formula (Ic), by reacting a compound of formula (IIIa) with a compound of the formula:

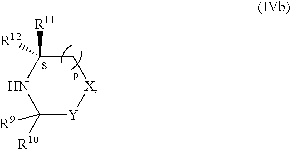

where the substituents have the values defined for formulae (Ib) and (Ic) above.

The reaction is preferably carried out in the presence of a base such as potassium carbonate, in an organic solvent such as a polar aprotic solvent, for example, acetonitrile, at a temperature of from 20° C. to 100° C. Examples of suitable leaving groups are mesylate, tosylate, triflate, chloride, bromide and iodide.

It will be appreciated that compounds of formulae (Ib) and (Ic) can also be produced by the preparation of compounds of formula (Ia) as the racemic mixture, followed by the separation of the corresponding isomers.

Intermediates of formula (IVa) wherein —X—Y is —N(Z)-CH₂— can be synthesised via reaction of the unprotected piperazine of the formula

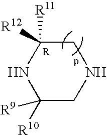

with a compound of formula Z-Lⁱⁱⁱ.

Such reactions are usually carried out in the presence of a palladium catalyst such as palladium acetate, BINAP ((R)-2,2′-bis(diphenylphosphino)-1,1′-binaphthyl) and a base such as Cesium carbonate.

In the same way intermediates of formula (IVb) wherein —X—Y is —N(Z)-CH₂— can be synthesised via reaction of the unprotected piperazine of the formula

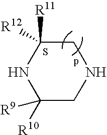

with a compound of formula Z-Lⁱⁱⁱ, using the same reaction conditions as described above.

Methods for then preparation of intermediates of formula (IVa) wherein —X—Y is —N(Z)-CH₂— are further illustrated below. Said methods refer to compounds wherein Z has different values and has several substitution patterns. These methods can be used for the preparation of a variety of intermediates of formula (IVa) depending on the starting materials and can equally be used to obtain the corresponding intermediates of formula (IVb). They all use the common step of reaction of the unprotected piperazine with a compound of formula Z-Lⁱⁱⁱ using the conditions described above unless stated otherwise.

Method a)

Intermediates of formula (IVa) wherein Z is (xii)ₐ and R¹⁶ is CN can be prepared as shown in the scheme below

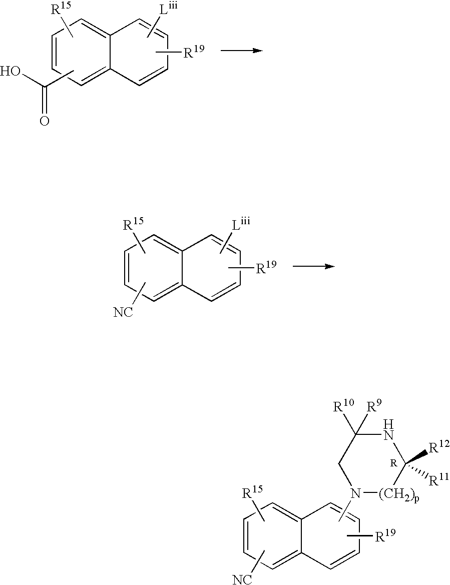

via conversion of the naphthoic acid into the corresponding naphthonitrile, followed by reaction with the unprotected piperazine as described above. The last reaction is preferably carried out in a solvent such as toluene and in the presence of a Palladium catalyst such as tris(dibenzylideneacetone)dipalladium(0), (R)-2,2′-bis(diphenylphosphino)-1,1′-binaphthyl (BINAP), and a base such as sodium tert-butoxide.

The acid moiety is converted to the nitrile using general methods known in the art, for example the reaction can be carried out in the presence of an activating reagent such as methanesulfonyl chloride and reacting the reactive intermediate with ammonia in an organic solvent such as pyridine. Further addition of methanesulfonyl chloride dehydrates the intermediate carboxamide to the nitrile.

Method b)

Intermediates of formula (IVa) wherein Z is (xii)ₐ and R¹⁹ is Cl can be prepared as shown in the scheme below:

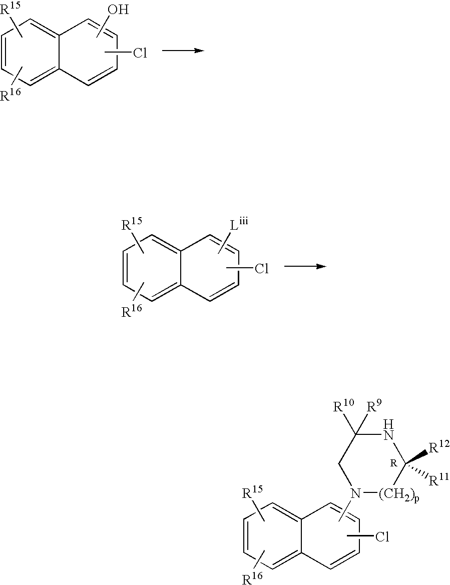

via conversion of the alcohol into a suitable leaving group Lⁱⁱⁱ followed by reaction with the unprotected piperazine as described above. When the Liii group is a triflate the reaction can for example be carried out in an organic solvent such as THF in the presence of a base such as sodium tert-butoxide and a triflating agent such as for example N-phenyltrifluoromethanesulfonimide.

Method c)

Intermediates of formula (IVa) wherein Z is (xii)ₐ and R¹⁹ is CN can also be prepared as shown in the scheme below:

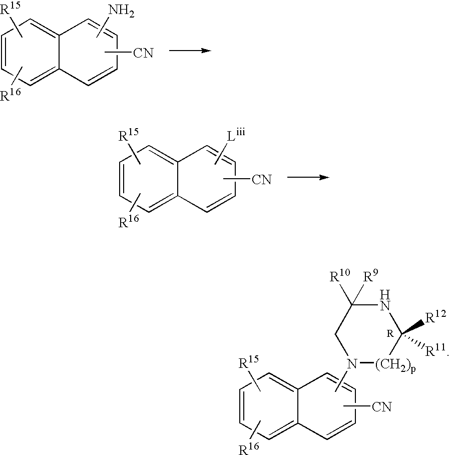

via conversion of the amino group of the corresponding aminonaphthonitrile into a suitable leaving group Lⁱⁱⁱ, followed by reaction with the unprotected piperazine as described above. When the Lⁱⁱⁱ group is a halide the reaction can for example be carried out in the presence of copper(I)halide and nitrous acid, such as a mixture of aqueous sodium nitrite and an acid such as hydrochloric acid.

Method d)

Intermediates of formula (IVa) wherein Z is (xii)ₐ and R¹⁶ and R¹⁹ are both F can be prepared as shown in the scheme below:

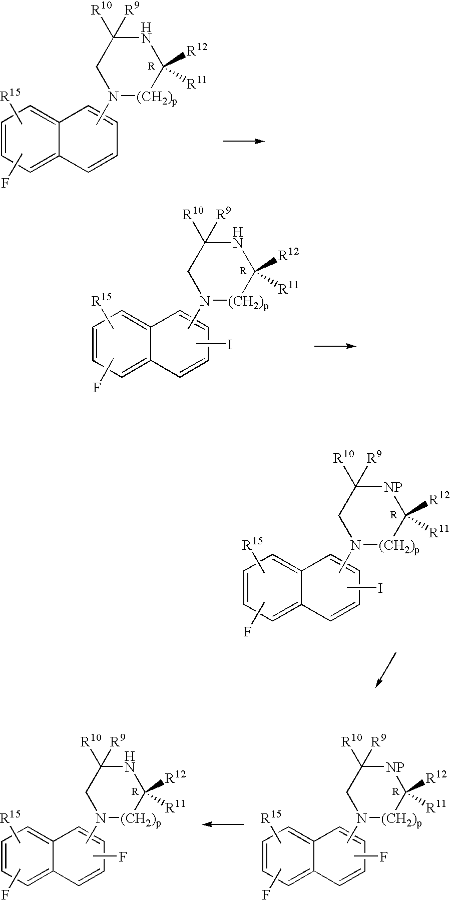

An iodo group is introduced into the napthalene ring, followed by protection of the nitrogen atom with a suitable protecting group P, conversion of the iodo group to a fluoro group and final deprotection.

The introduction of the iodo group is preferably carried out using general iodination conditions such as in the presence of a mixture of bis(pyridine)iodonium(I) tetrafluoroborate and tetrafluoroboric acid in an organic solvent such as dichloromethane.

The nitrogen atom can be protected using general conditions as those shown in Greene and Wuts, Protecting Groups in Organic Synthesis, 3rd. Ed., John Wiley & Sons and a suitable protecting group is for example CBZ. Said protecting groups can be cleaved following the procedures also described in Greene and Wuts, Protecting Groups in Organic Synthesis, 3rd. Ed., John Wiley & Sons.

The iodo group is converted to a fluoro group in the presence of N-fluorobenzenesulfonimide and a base such as tert-butillithium in an organic solvent such as tetrahydrofuran.

Intermediates of formula (IVa) wherein Z is (xii)ₐ and R¹⁶ is F and R¹⁹ is CN can be prepared as shown in the scheme below:

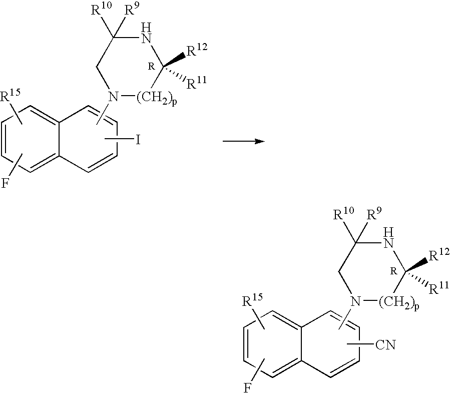

via conversion of the iodo group into the corresponding nitrile group.

The reaction is preferably carried out in the presence of a cyanide such as potassium cyanide, a catalyst such as copper (I) iodide and a palladium catalyst such as tetrakis(triphenylphosphine)palladium (0) in an organic solvent such as tetrahydrofuran. The reaction mixture is preferably heated for example at a temperature around 100° C.

Method e)

Intermediates of formula (IVa) wherein Z is (xii)ₐ and R¹⁶ and R¹⁹ are both methyl can be prepared as shown in the scheme below:

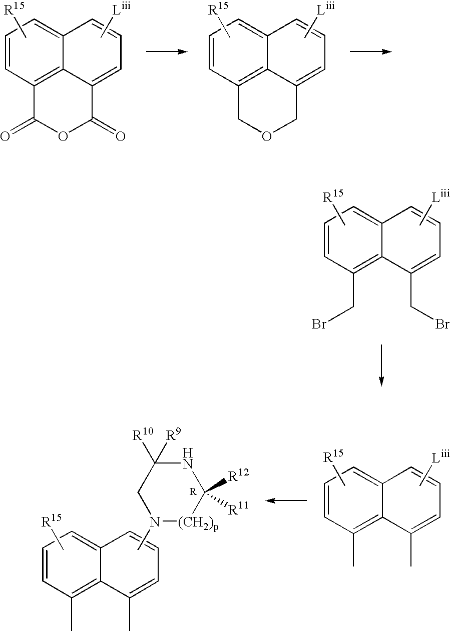

wherein the 1H,3H-naphtho[1,8-cd]pyran-1,3-diones is reduced to the corresponding 1H,3H-naphtho[1,8-cd]pyran, then the pyran ring of the 1H,3H-naphtho[1,8-cd]pyran is opened to give the corresponding bis(bromomethyl)naphthalene derivative, which is subsequently converted to the dimethyl compound, followed by reaction with the corresponding unprotected piperazine as described above.

The reduction is preferably carried out in the presence of a reducing agent such as sodium borohydride in an organic solvent such as ethanol, followed by reaction with an acid such as trifluoroacetic acid in an organic solvent such as dichloromethane and in the presence of a reducing agent such as triethylsilane.

The pyran ring is preferably opened in the presence of a reagent such as boron tribromide in an organic solvent such as dichloromethane at reflux.

The dimethyl compound is preferably prepared in the presence of a reducing agent such as sodium borohydride, in the presence of an activating agent such as silver nitrate, in an organic solvent such as dimethylformamide.

Method f)

Intermediates of formula (IVa) wherein Z is (xii)ₐ and R¹⁶ is F can be prepared as shown in the scheme below:

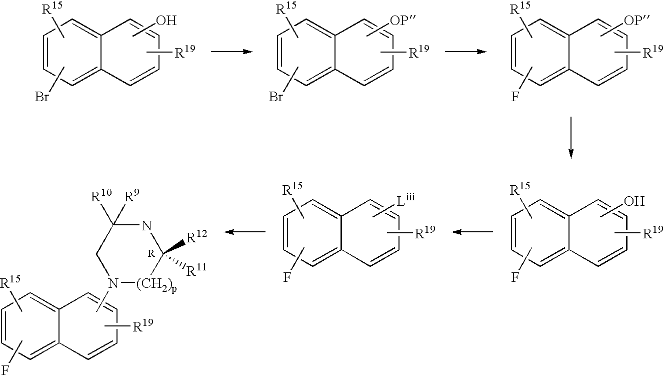

wherein the naphthol compound is protected with a suitable alcohol protecting group P″, as those described in Greene and Wuts, Protecting Groups in Organic Synthesis, 3rd. Ed., John Wiley & Sons, followed by conversion of the bromo group into a fluoro group, deprotection of the alcohol and conversion into a suitable leaving group Lⁱⁱⁱ, then by reaction with the corresponding unprotected piperazine as described above.

The alcohol can be protected using general conditions as those shown in Greene and Wuts, Protecting Groups in Organic Synthesis, 3rd. Ed., John Wiley & Sons and a suitable protecting group is for example tert-butyldimethylsilyl. Said protecting groups can be cleaved following the procedures also described in Greene and Wuts, Protecting Groups in Organic Synthesis, 3rd. Ed., John Wiley & Sons.

The bromo group is converted to a fluoro group in the presence of N-fluorobenzenesulfonimide and a base such as tert-butillithium in an organic solvent such as tetrahydrofuran.

The conversion of the alcohol into a suitable leaving group such as a triflate can be carried out in an organic solvent such as THF in the presence of a base such as sodium tert-butoxide and a triflating agent such as for example N-phenyltrifluoromethanesulfonimide.

Method g)

Intermediates of formula (IVa) wherein Z is (xii)ₐ and R¹⁶ is Cl can also be prepared as shown in the scheme below:

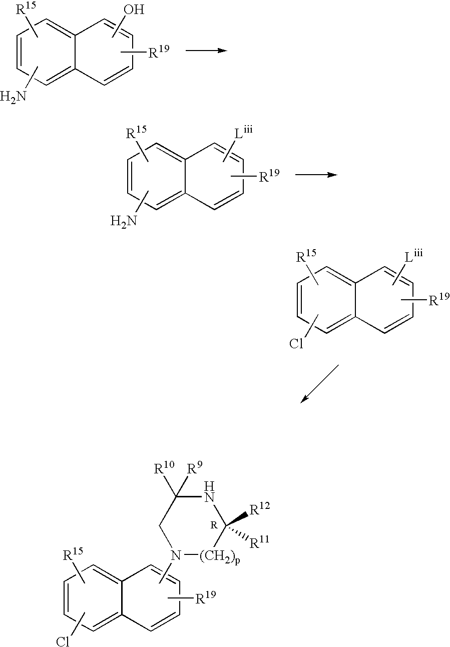

via conversion of the alcohol into a suitable leaving group Lⁱⁱⁱ, the amino group into a chloro group, followed by reaction with the unprotected piperazine as described above. When the Liii group is a triflate the first reaction can for example be carried out in an organic solvent such as THF in the presence of a base such as sodium tert-butoxide and a triflating agent such as for example N-phenyltrifluoromethanesulfonimide.

The amino group is preferably reacted with copper(I)chloride and nitrous acid, such as a mixture of aqueous sodium nitrite and an acid such as hydrochloric acid.

Method h)

Intermediates of formula (IVa) wherein Z is (xii)ₐ and R¹⁶ is CN can also be prepared as shown in the scheme below:

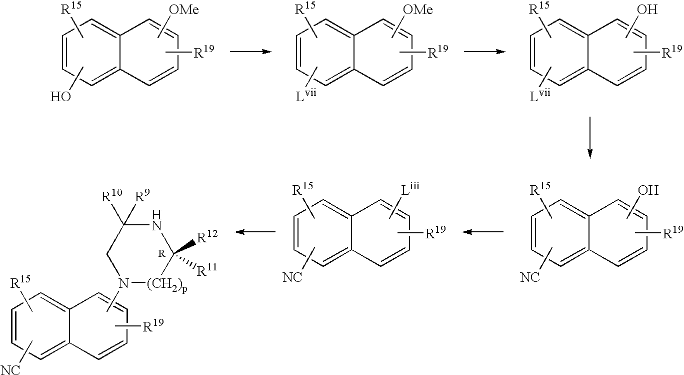

via conversion of the alcohol into a suitable leaving group L^{vii}, deprotection of the ether to give an alcohol, displacement of L^{vii} with a nitrile group, conversion of the alcohol into a suitable leaving group Lⁱⁱⁱ, followed by reaction with the unprotected piperazine as described above.

The conversions of the alcohol into suitable leaving groups Lⁱⁱⁱ and L^{vii}, when the Lⁱⁱⁱ and L^{vii} groups are triflates can for example be carried out in an organic solvent such as THF in the presence of a base such as sodium tert-butoxide and a triflating agent such as for example N-phenyltrifluoromethanesulfonimide.

The methyl ether is deprotected with boron tribromide in a suitable organic solvent such as dichloromethane.

The displacement of L^{vii} with a nitrile group is preferably carried out by heating the compound in a suitable organic solvent such as DMF, in the presence of a cyanide such as for example zinc cyanide and a palladium catalyst such as tetrakis triphenylphosphine palladium (0).

Method i)

Intermediates of formula (IVa) wherein Z is (i)ₐ and R¹⁶ is CN can be prepared as shown in the scheme below:

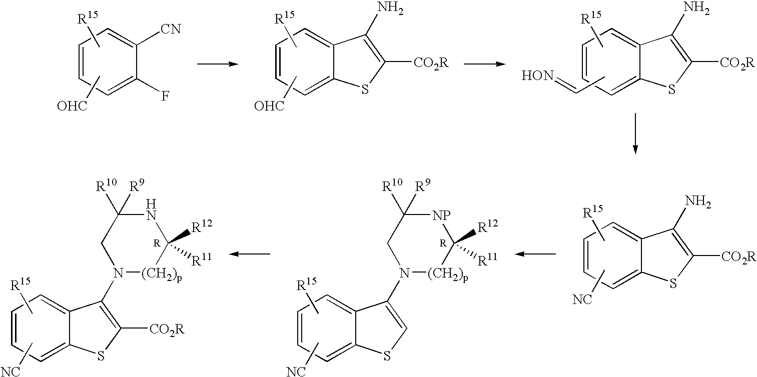

via formation of the benzothiophene ring, conversion of the formaldehyde into nitrile, and displacement of the 3-amino group with a monoprotected piperazine and concomitant decarboxylation of the benzothiophene. The piperazine is protected using general conditions as those shown in Greene and Wuts, Protecting Groups in Organic Synthesis, 3rd. Ed., John Wiley & Sons, a suitable protecting group being for example the trifluoroacetamide.

The benzothiophene ring is preferably formed by heating the fluoroformylbenzonitrile compound in the presence of ethyl thioglycolate and a base such as triethylamine in an organic solvent such as DMSO.

The conversion of the formaldehyde into the corresponding nitrile is carried out via formation of the corresponding hydroxylimine in the presence of hydroxylamine hydrochloride in a suitable organic solvent such as acetonitrile and a suitable base such as triethylamine.

Method j)

Intermediates of formula (IVa) wherein Z is (xiii) can be prepared as shown in the scheme below:

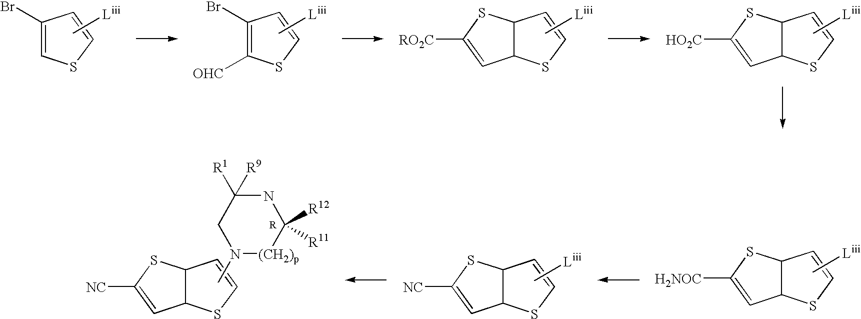

via insertion of a carbaldehyde group into the thiophene ring, formation of the bromothieno[3,2-b]thiophene, to the carboxylic acid, to the carboxamide, then a nitrile and reaction with the unprotected piperazine using the conditions above.

The carbaldehyde is preferably inserted in a suitable solvent such as tetrahydrofuran, in the presence of dimethylformamide and a base such as lithium diisopropylamide.

The formation of the bromothieno[3,2-b]thiophene is preferably carried out in a mixture of solvents such as DMSO and acetonitrile, in the presence of an alkyl thioglycolate and a suitable base such as triethylamine. The thieno[3,2-b]thiophene-2-carboxylate compound is preferably saponified in basic conditions such as for example aqueous sodium hydroxide in a suitable solvent such as ethanol under reflux.

The thieno[3,2-b]thiophene-2-carboxamide is preferably prepared in the presence of Ammonia, a coupling reagent such as carbonyl diimidazole and a base such as triethylamine in a suitable solvent such as THF. The thieno[3,2-b]thiophene-2-carbonitrile is preferably formed by dehydration of the carboxamide with for example methanesulfonyl chloride in the presence of a suitable base such as pyridine.

Method k)

Intermediates of formula (IVa) wherein —X—Y— is

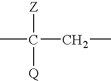

Q is hydrogen and for example Z is (xii)ₐ can be prepared as shown in the scheme below:

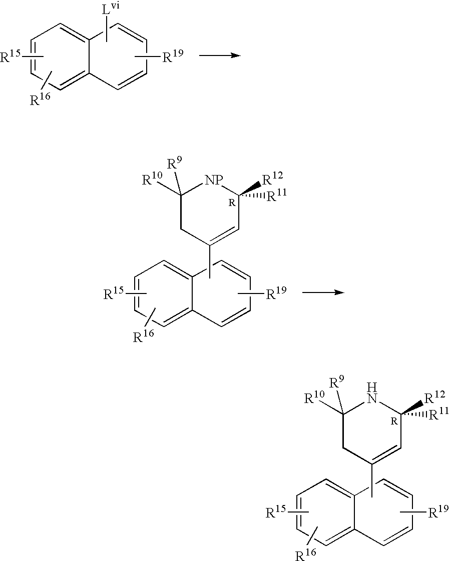

via reaction of the Z-Lvi compound wherein Lvi is a suitable leaving group such as triflate with a N-protected 4-(4,4,5,5-tetramethyl-1,3,2-dioxaborolan-2-yl)-3,6-dihydro-1(2H)-pyridine, (which can be synthesised according to the procedure described by Paul R. Eastwood in Tetrahedron Letters, 2000, 41, 3705-3708) in the presence of a base such as potassium carbonate and a palladium catalyst such as bis(diphenylphosphino)-ferrocenedichloropalladium(II) in a suitable solvent such as DMF to give the corresponding protected 3,6-dihydro-1(2H)-pyridine, which is converted to the corresponding protected piperidine, and then deprotected.

The piperidine compound can be prepared by reduction with hydrogen in the presence of a palladium catalyst such as palladium on carbon in a suitable solvent such as methanol.

The deprotection of the piperidine can be carried out according to the nitrogen-protecting group (P) used. Suitable protecting groups are shown in Greene and Wuts, Protecting Groups in Organic Synthesis, 3rd. Ed., John Wiley & Sons and include tert-Butylcarboxylate (BOC) and can be deprotected for example in a suitable solvent such as dichloromethane and in the presence of trifluoroacetic acid.

Method l)

Intermediates of formula (IIIa) wherein R¹ is

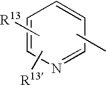

can be prepared from the corresponding protected alcohols of formula (Va) via deprotection following suitable conditions as those described in Greene and Wuts, Protecting Groups in Organic Synthesis, 3rd. Ed., John Wiley & Sons. Suitable protecting groups (P′) are also described in the above reference and include tertbutyldimethylsilyl groups. Said alcohols of formula (Va) can be prepared as shown in the scheme below:

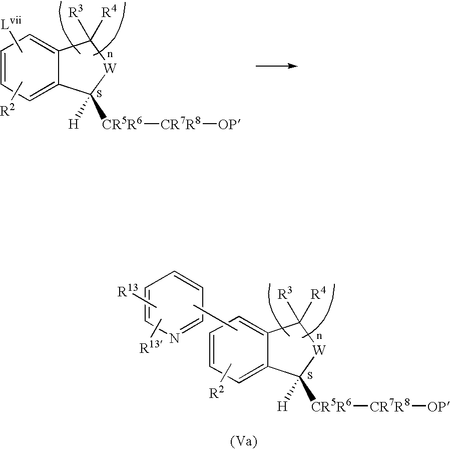

wherein L^{vii} is a suitable leaving group such as Bromo, via reaction with the corresponding dioxaborinanyl pyridine. This reaction is preferably carried out in the presence of a suitable solvent such as toluene and in the presence of a palladium catalyst such as tetrakis(triphenylphosphine) palladium and a suitable base such as potassium hydroxide.

Method m)

Intermediates of formula (IIIa) wherein R¹ is SO₂NR¹³R¹⁴ can be prepared from the corresponding protected alcohols of formula (Vb) via deprotection following suitable conditions as those described in Greene and Wuts, Protecting Groups in Organic Synthesis, 3rd. Ed., John Wiley & Sons. Suitable protecting groups (P′) are also described in the above reference and include tertbutyldimethylsilyl groups. Said alcohols of formula (Vb) can be prepared as shown in the scheme below:

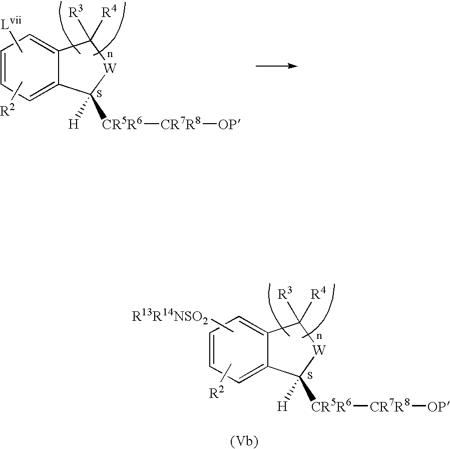

wherein L^{vii} is a suitable leaving group such as Bromo, via formation of the corresponding sulfonamide. This reaction is preferably carried out in a suitable solvent such as tetrahydrofuran and in the presence of sulphur dioxide and a suitable base such as n-butyllithium, followed by reaction in a suitable solvent such as dichloromethane in the presence of N-chlorosuccinimide and the corresponding amine (HNR¹³R¹⁴).

As shown above substituents in any of the aromatic ring, such as R¹ and R², may be present in the starting materials or introduced at an appropriate point in the manufacture of the product compound. If necessary said substituents may be protected during the reaction procedure.

Compounds of the invention have been demonstrated to be active at the serotonin, 5-HT 1D receptor. Their binding activity has been demonstrated in a tests described by Pullar I. A. et al, European Journal of Pharmacology, 407 (2000), 39-40.

As mentioned above, the compounds of the invention and their pharmaceutically acceptable salts have useful central nervous system activity. They have been shown to increase release of tritiated-5HT from guinea pig cortical slices in a test with the following procedure.

Cortical slices from the brains of male guinea pigs were incubated with 50 nM [³H]-5-HT for 30 minutes at 37° C. The slices were washed in basal buffer containing 1 μM paroxetine and then transferred to baskets. The baskets were used to transfer the tissue between the washing and release buffers, all of which contained 1 μM paroxetine.

In order to obtain a stable baseline release, the slices were incubated for 11 minutes in buffer and then transferred for 4 minutes to a second tube containing buffer. Following incubation they were again transferred, for a further 4 minutes, to a buffer in which NaCl had been substituted, on an equimolar basis, to give a KCl concentration of 30 mM (release sample).

The tritium in the tissue samples and in the buffers from the three incubation periods was estimated by liquid scintillation spectroscopy. Test compound was present throughout the three incubation periods. The compounds of the invention enhanced release of 5-HT.

The compounds of the invention are serotonin reuptake inhibitors, and possess excellent activity as, for example, in the test described by Carroll et al., J. Med. Chem. (1993), 36, 2886-2890, in which the intrinsic activity of the compound to competitively inhibit the binding of selective serotonin reuptake inhibitors to the serotonin transporter is measured. These results were also confirmed by in vivo tests in which the effect of the compound on a behavioural syndrome in mice dosed with 5-HTP and a monoamine oxidase inhibitor (MAOI) such as pargyline, is measured, see Christensen, A. V., et al., Eur. J. Pharmacol. 41, 153-162 (1977).

In view of the selective affinity of the compounds of the invention for the serotonin receptors, they are indicated for use in treating a variety of conditions such as depression, bipolar disorder, anxiety, obesity, eating disorders such as anorexia and bulimia, alcoholism, pain, hypertension, ageing, memory loss, sexual dysfunction, psychotic disorders, schizophrenia, gastrointestinal disorders, headache, cardiovascular disorders, smoking cessation, epilepsy, drug abuse and addiction, emesis, Alzheimer's disease and sleep disorders. The compounds of the invention are principally intended for the treatment of depression or anxiety, or disorders with depressive or anxiety symptoms.

The compounds of the invention are effective over a wide dosage range, the actual dose administered being dependent on such factors as the particular compound being used, the condition being treated and the type and size of animal being treated. However, the dosage required will normally fall within the range of 0.001 to 20, such as 0.01 to 20 mg/kg per day, for example in the treatment of adult humans, dosages of from 0.5 to 100 or 200 mg per day may be used.

The compounds of the invention will normally be administered orally or by injection and, for this purpose, the compounds will usually be utilised in the form of a pharmaceutical composition. Such compositions are prepared in a manner well known in the pharmaceutical art and comprise at least one active compound.

Accordingly the invention includes a pharmaceutical composition comprising as active ingredient a compound of formula (I) or a pharmaceutically acceptable salt thereof, associated with a pharmaceutically acceptable diluent or carrier. In making the compositions of the invention, the active ingredient will usually be mixed with a carrier, or diluted by a carrier, or enclosed within a carrier which may be in the form of a capsule, sachet, paper or other container. More than one active ingredient or excipient may, of course, be employed. The excipient may be a solid, semi-solid or liquid material which acts as a vehicle, excipient or medium for the active ingredient. Some examples of suitable excipients are lactose, dextrose, sucrose, sorbitol, mannitol, starches, gum acacia, calcium phosphate, alginates, tragacanth, gelatin, syrup, methyl cellulose, methyl- and propyl-hydroxybenzoate, talc, magnesium stearate or oil. The compositions of the invention may, if desired, be formulated so as to provide quick, sustained or delayed release of the active ingredient after administration to the patient.

Depending on the route of administration, the foregoing compositions may be formulated as tablets, capsules or suspensions for oral use and injection solutions or suspensions for parenteral use or as suppositories. Preferably the compositions are formulated in a dosage unit form, each dosage containing from 0.5 to 100 mg, more usually 1 to 100 mg, of the active ingredient.

The following Preparations and Examples illustrate routes to the synthesis of the compounds of the invention.

Preparation

### 1-(2-{[tert-butyl(dimethyl)silyl]oxy}ethyl)-3,4-dihydro-1H-2-benzopyran-6-carboxamide

Method A

### a) 2-(6-Bromo-3,4-dihydro-1H-2-benzopyran-1-yl)ethanol

Lithium borohydride (2.9 g, 133 mmol) was added over one hour to a stirred solution of ethyl (6-bromo-3,4-dihydro-1H-2-benzopyran-1-yl)acetate (log, 33.4 mmol) in THF (250 mL) under a nitrogen atmosphere. After stirring overnight, water (100 mL) was added cautiously and, after stirring for 1 h, the product extracted into ethyl acetate. The organic extracts were washed with water, dried (MgSO₄), and evaporated in vacuo to give the title compound as an oil.

b) 2-(6-Bromo-3,4-dihydro-1H-2-benzopyran-1-yl)ethyl tert-butyl(dimethyl)silyl ether

A 1M solution of tert-butyldimethylsilyl chloride in dichloromethane (30 mL, 30 mmol) was added dropwise under nitrogen to an ice/water-cooled solution of 2-(6-bromo-3,4-dihydro-1H-2-benzopyran-1-yl)ethanol (6.7 g, 24.4 mmol), diisopropylethylamine (6.7 g. 51.8 mmol) and dimethylaminopyridine (0.32 g, 2.5 mmol) in dry dimethylformamide (70 mL). After stirring overnight at room temperature, the mixture was quenched with ice/water and extracted with ether (2×). The combined organic extracts were washed with water (5×), dried (MgSO₄) and evaporated in vacuo to give an oil. This was purified by flash chromatography on silica, eluting with ethyl acetate/hexane (0:100 to 10:90), to give the title compound as oil.

### c) 1-(2-{[tert-Butyl(dimethyl)silyl]oxy}ethyl)-3,4-dihydro-1H-2-benzopyran-6-carboxamide

A 1.7M solution of tert-butyllithium in pentane (17.5 mL, 29.7 mmol) was added under nitrogen to a solution of 2-(6-bromo-3,4-dihydro-1H-2-benzopyran-1-yl)ethyl tert-butyl(dimethyl)silyl ether (5 g, 14.0 mmol) in dry tetrahydrofuran (80 mL), maintained at −70° C. After 45 min at −70° C., trimethylsilylisocyanate (2.5 g, 21.7 mmol) in tetrahydrofuran (10 ml) was added dropwise, then the reaction mixture was allowed to warm to room temperature overnight. A solution of saturated ammoniun chloride in water was added and, after 15 min, the solution basified with 2N sodium hydroxide. The product was extracted into dichloromethane, dried (MgSO₄) and evaporated in vacuo to give a oil (4.8 g). This was purified by flash chromatography on silica, eluting with ethyl acetate/hexane (0:100 to 100:0), then methanol/ethyl acetate (10:90), to give the title compound as a solid.

Method B

### a) 1-(2-{[tert-Butyl(dimethyl)silyl]oxy}ethyl)-3,4-dihydro-1H-2-benzopyran-6-carboxylic acid

A 1.7M solution of tert-butyl lithium in pentane (1.75 mL, 2.97 mmol) was added under nitrogen to a solution of 2-(6-bromo-3,4-dihydro-1H-2-benzopyran-1-yl)ethyl tert-butyl(dimethyl)silyl ether (0.5 g, 1.35 mmol) in tetrahydrofuran (10 mL), maintained at −70° C. After 35 min, carbon dioxide was bubbled through the reaction mixture for 35 min. After stirring at room temperature overnight, saturated ammonium chloride in water was added and the product extracted into ethyl acetate. The organic extracts were dried (MgSO₄) and evaporated in vacuo to give an oil (0.57 g). This was purified by flash chromatography on silica, eluting with ethyl acetate/hexane (0:100 to 25:75) to give the title compound as a white solid.

### b) 1-(2-{[tert-Butyl(dimethyl)silyl]oxy}ethyl)-3,4-dihydro-1H-2-benzopyran-6-carboxamide

A solution of 1-(2-{[tert-butyl(dimethyl)silyl]oxy}ethyl)-3,4-dihydro-1H-2-benzopyran-6-carboxylic acid (20.7 g, 61.2 mmol) and 1,1′-cabonyldiimidazole (20 g, 123 mmol) in dry tetrahydrofuran (450 mL) was stirred under nitrogen at room temperature for 16 h. A 0.5M solution of ammonia in dioxane (620 mL, 310 mmol) was added and the mixture stirred at room temperature for 1 day. Water (1 L) was added and the product extracted into dichloromethane (2×1 L). The combined organic extracts were washed with saturated aqueous sodium bicarbonate (2×500 mL) and brine (2×500 mL), dried (MgSO₄) and evaporated in vacuo to give a solid (21 g). This was purified by flash chromatography on silica , eluting with hexane/ethyl acetate (1:1) then ethyl acetate to give the title compound.

### EXAMPLE 1

### 1-[2-(4-(6-Fluoro-1H-indol-3yl-)-3,6-dihydro-1(2H)-pyridinyl)ethyl]-3,4-dihydro-1H-2-benzopyran-6-carboxamide

### a) 1-(2-Hydroxyethyl)-3,4-dihydro-1H-2-benzopyran-6-carboxamide

1-(2-{[tert-Butyl(dimethyl)silyl]oxy}ethyl)-3,4-dihydro-1H-2-benzopyran-6-carboxamide (1 g, 2.98 mmol) was dissolved in a mixture of acetic acid (10 mL) and water (5 mL), then stirred for 2 h. The solution was evaporated to give a residue that was dried in vacuo at 55° C. to give the title compound as a white solid.

### b) 2-[6-(Aminocarbonyl)-3,4-dihydro-1H-2-benzopyran-1-yl]ethyl methanesulfonate

1-(2-Hydroxyethyl)-3,4-dihydro-1H-2-benzopyran-6-carboxamide (5 g, 22.6 mmol) was dissolved in a mixture of dry tetrahydrofuran (375 mL) and dry dimethylformamide (15 mL) with the aid of gentle heating. Triethylamine (4.6 g, 45.5 mmol) was added, followed by methanesulfonyl chloride (2.72 g, 23.8 mmol). The mixture was stirred under nitrogen at room temperature for 1 day. The reaction mixture was quenched with water (1000 mL) and the product extracted into ethyl acetate (2×500 mL). The combined organic extracts were washed with brine (2×500 mL), dried (MgSO₄), and evaporated in vacuo to give the crude product as a white solid (6.5 g, 97%). The solid was triturated with ether (300 mL) to give 2-[6-(aminocarbonyl)-3,4-dihydro-1H-2-benzopyran-1-yl]ethyl methanesulfonate as a white solid.

### c) 1-[2-(4-(6-Fluoro-1H-indol-3yl-)-3,6-dihydro-1(2H)-pyridinyl)ethyl]-3,4-dihydro-1H-2-benzopyran-6-carboxamide

6-Fluoro-3-(1,2,3,6-tetrahydropyridin-4-yl)-1H-indole (1.1 g, 5.0 mmol), 2-[6-(aminocarbonyl)-3,4-dihydro-1H-2-benzopyran-1-yl]ethyl methanesulfonate (1.3 g, 4.35 mmol), potassium carbonate (1.8 g, 13 mmol), potassium iodide (0.07 g, 0.42 mmol) and acetonitrile (60 mL) were heated under reflux for 1 day with stirring under nitrogen. After cooling to room temperature, water (60 mL) was added and the product extracted into ethyl acetate. The combined organic extracts were washed with brine, dried (MgSO₄) and evaporated in vacuo to give the crude product as a yellow oil (2 g). This was purified by flash chromatography on silica, eluting with ethyl acetate, then with increasing amounts of methanol (1:99 to 50:50) in ethyl acetate, to give the title compound as a yellow solid (mp 110.0-121.9° C.). M+H=420.

The following Examples were made by substituting the 6-fluoro-3-(1,2,3,6-tetrahydropyridin-4-yl)-1H-indole in the above example with alternative secondary amines as indicated in each example below:

### EXAMPLE 2

### 1-[2-(4-(1H-Indol-3yl-)-3,6-dihydro-1(2H)-pyridinyl)ethyl]-3,4-dihydro-1H-2-benzopyran-6-carboxamide

Prepared from 3-(1,2,3,6-tetrahydropyridin-4-yl)-1H-indole. M+H=402.

### EXAMPLE 3

### 1-[2-(4-(5-Fluoro-1H-indol-3yl-)-3,6-dihydro-1(2H)-pyridinyl)ethyl]-3,4-dihydro-1H-2-benzopyran-6-carboxamide

Prepared from 5-fluoro-3-(1,2,3,6-tetrahydropyridin-4yl)-1H-indole. M+H=420.

### EXAMPLE 4

### 1-(2-{4-[2-(1H-Indol-3-yl)ethyl]-1-piperidinyl}ethyl)-3,4-dihydro-1H-2-benzopyran-6-carboxamide

Prepared from 3-[2-(4-piperidinyl)ethyl]-1H-indole. M+H=432.

### EXAMPLE 5

### 1-[2-(4-(6H-Thieno[3,2]pyrrol-6-yl)-3,6-dihydro-1(2H)-pyridinyl)ethyl]-3,4-dihydro-1H-2-benzopyran-6-carboxamide

Prepared from 4-(thieno[3,2-b]pyrrol-6-yl)-1,2,3,6-tetrahydropyridine:. M+H=408.

### EXAMPLE 6

### 1-{2-[(2R)-2-Methyl-4-(1-naphthyl)piperazinyl]ethyl}-3,4-dihydro-1H-2-benzopyran-6-carboxamide

Prepared from (3R)-3-methyl-1-(1-naphthyl)piperazine. M+H=430.

### EXAMPLE 7

### 1-{2-[(2S)-2-Methyl-4-(1-naphthyl)piperazinyl]ethyl}-3,4-dihydro-1H-2-benzopyran-6-carboxamide

Prepared from (3S)-3-methyl-1-(1-naphthyl)piperazine. M+H=430.

### EXAMPLE 8

### 1-{2-[-4-(6-Fluoro-1-naphthyl)piperazinyl]ethyl)-3,4-dihydro-1H-2-benzopyran-6-carboxamide

Prepared from 1-(6-fluoro-1-naphthyl)piperazine. M+H=434.

### EXAMPLE 9

### 1-{2-[-4-(7-Fluoro-1-naphthyl)piperazinyl]ethyl}-3,4-dihydro-1H-2-benzopyran-6-carboxamide

Prepared from 1-(7-fluoro-1-naphthyl)piperazine. M+H=434.

### EXAMPLES 10 and 11

### 1-[2-(4-(6-Fluoro-7-methyl-1H-indol-3-yl)-3,6-dihydro-1(2H)-pyridinyl)ethyl]-3,4-dihydro-1H-2-benzopyran-6-carboxamide and 1-[2-(4-(6-fluoro-7-methyl-1H-indol-3-yl)-3,6-dihydro-1(2H)-pyridinyl)ethyl]-3,4-dihydro-1H-2-benzopyran-6-carbonitrile

Methanesulphonyl chloride (0.14 g, 1.2 mmol) was added under nitrogen to a mixture of 2-[6-(aminocarbonyl)-3,4-dihydro-1H-2-benzopyran-1-yl]ethyl methanesulfonate (0.3 g, 1 mmol) and triethylamine (0.3 g, 3 mmol) in tetrahydrofuran (10 mL). After stirring at room temperature overnight water was added and the product extracted into ethyl acetate. The combined organic extracts were washed (water), dried (MgSO₄) and evaporated in vacuo to give the crude product as an oil (0.3 g), containing a mixture of 2-[6-(cyano)-3,4-dihydro-1H-2-benzopyran-1-yl]ethyl methanesulfonate and unreacted 2-[6-(aminocarbonyl)-3,4-dihydro-1H-2-benzopyran-1-yl]ethyl methanesulfonate, which was used directly in the next step below.

The oil above was coupled with 6-fluoro-7-methyl-3-(1,2,3,6-tetrahydro-4-pyridinyl)-1H-indole, using conditions described above, to give the required product 1-[2-(4-(6-fluoro-7-methyl-1H-indol-3-yl)-3,6-dihydro-1(2H)-pyridinyl)ethyl]-3,4-dihydro-1H-2-benzopyran-6-carbonitrile (M+H=416) and 1-[2-(4-(6-fluoro-7-methyl-1H-indol-3-yl)-3,6-dihydro-1(2H)-pyridinyl)ethyl]-3,4-dihydro-1H-2-benzopyran-6-carboxamide (M+H=434).

### EXAMPLE 12

### 1-{2-[4-(5-Methoxy-1H-indol-3-yl)-1-piperidinyl]ethyl}-3,4-dihydro-1H-2-benzopyran-6-carboxamide

### a) tert-Butyl 4-(5-methoxy-1H-indol-3-yl)-3,6-dihydro-1(2H)-pyridinecarboxylate

To a solution of 5-methoxy-1H-indole (3 g, 20.4 mmol) in tert-butanol (150 mL), cooled in an ice water bath, were added N-BOC-piperidone (6.1 g, 30.6 mmol) and potassium tert-butoxide (9.2 g, 82 mmol) and the resultant mixture heated at reflux under nitrogen overnight. The mixture was poured into water and the product extracted into ethyl acetate. The combined organic extracts were washed (water), dried (MgSO₄) and evaporated in vacuoto. The crude oil was purified by flash chromatography on silica, eluting with ethyl acetate/hexane (0:100 to 50:50), to give the title compound as a glass.

### b) tert-Butyl 4-(5-methoxy-1-methyl-1H-indol-3-yl)-3,6-dihydro-1(2H)-pyridinecarboxylate

Sodium hydride (50% oil dispersion) was added at 0° C. under nitrogen to tert-butyl 4-(5-methoxy-1H-indol-3-yl)-3,6-dihydro-1(2H)-pyridinecarboxylate (2 g, 6.1 mmol) in dry DMF (20 mL). After stirring for 1 h at room temperature, iodomethane (1.73 g, 12.2 mmol) was added. After stirring overnight, the mixture was evaporated in vacuo, water added and the mixture extracted with ethyl acetate. The combined organic extracts were washed with water, dried (MgSO₄) and evaporated in vacuo. The resultant oil was purified by flash chromatography on silica, eluting with ethyl acetate/hexane (0:100 to 100:0), to give the title compound as a glass.

### c) 5-Methoxy-1-methyl-3-(4-piperidinyl)-1H-indole

tert-Butyl 4-(5-methoxy-1-methyl-1H-indol-3-yl)-3,6-dihydro-1(2H)-pyridinecarboxylate (0.22 g, 0.64 mmol)and 5% Pd/C (70 mg) in ethanol (50 mL) were hydrogenated at 60 psi in a Parr hydrogenator for 2 h. The catalyst was filtered off and the solvent removed in vacuo to give tert-butyl 4-(5-methoxy-1-methyl-1H-indol-3-yl)-1-piperidinecarboxylate (0.2 g). The crude oil was dissolved in a mixture of trifluoroacetic acid (1.1 mL) and dichloromethane (4 mL), and the solution stirred under nitrogen at room temperature for 1 h. The mixture was evaporated to dryness, then water added, followed by 2N sodium hydroxide until basic. The product was extracted into ethyl acetate, washed (water), dried (MgSO₄) and evaporated in vacuo to give the title compound as an oil.

### d) 1-{2-[4-(5-Methoxy-1-methyl-1H-indol-3-yl) -1-piperidinyl]ethyl}-3,4-dihydro-1H-2-benzopyran-6-carboxamide

A mixture of 5-methoxy-1-methyl-3-(4-piperidinyl)-1H-indole (79 mg, 0.33 mmol), 2-[6-(aminocarbonyl)-3,4-dihydro-1H-2-benzopyran-1-yl]ethyl methanesulphonate (0.12 g, 0.4 mmol), potassium carbonate (0.1 g, 0.72 mmol) and potassium iodide (1 mg, 0.006 mmol) in acetonitrile (2 mL) was heated at reflux for 36 h. Water was added and the product extracted into ethyl acetate. The combined organic extracts were washed (water), dried (MgSO₄) and evaporated in vacuo. The crude product was purified by flash chromatography on silica, eluting with ethyl acetate then methanol/ethyl acetate (10:90), to yield the title compound. M+H=448.

### EXAMPLE 13

### 1-{2-[(2S)-4-(6-Fluoro-1-benzothien-3-yl)-2-methylpiperazinyl]ethyl}-3,4-dihydro-1H-2-benzopyran-6-carboxamide

### a) [(3-Fluorophenyl)thio]acetic acid

A solution of 2-chloroacetic acid (7.4 g, 78 mmol) and 3-fluorobenzenethiol (10 g, 78 mmol) in 10% aqueous sodium hydroxide (100 mL) was stirred at room temperature for 10 min. The resultant precipitate was filtered off. Addition of solid citric acid to the mother liquors gave further precipitation of the product. The combined solids were dried in vacuo to give the title compound as a white solid.

### b) 6-Fluoro-1-benzothiophen-3 (2H) -one

To a stirred solution of [(3-fluoro-phenyl)thio)acetic acid (10 g, 53.7 mmol) in chloroform (100 mL) was added thionyl chloride (8.33 g, 70 mmol) dropwise. The reaction mixture was heated at reflux for 3 h, then cooled to 0° C. Excess aluminium trichloride (56.7 g, 500 mmol) was added cautiously and the resultant solution stirred for 14 h at room temperature. The mixture was poured onto ice-water and extracted into diethyl ether. The organic extracts were dried (MgSO₄), filtered and evaporated in-vacuo. The crude product was purified by elution with ethyl acetate through a short silica pad, to yield the title compound.

### c) 6-Fluoro-1-benzothiophene

To a stirred solution of 6-fluoro-1-benzothiophen-3(2H)-one (6.0 g, 35.7 mmol) in ethanol (120 mL) was added sodium borohydride (1.35 g, 35.7 mmol). After stirring for 1 h at room temperature, the solvent was removed in vacuo and the resultant red oil taken up in ethyl acetate and washed with water (2×). The combined organic extracts were dried (MgSO4), filtered and evaporated in vacuo. The crude solid was dissolved in chloroform (50 mL) and stirred as a few drops of 2M hydrochloric acid were added. After stirring for 30 min, the solution was washed with water, dried (MgSO₄), filtered and evaporated in vacuo. The crude product was purified by flash chromatography on silica, eluting with ethyl acetate/hexane (15:85) to yield the title compound.

### d) 3-Bromo-6-fluoro-1-benzothiophene

To a solution of 6-fluoro-1-benzothiophene (15 g, 98.7 mmol) in dimethylformamide (100 mL) was added N-bromosuccinimide (17.6 g, 98.7 mmol) and stirred at room temperature overnight. The reaction mixture was washed with water and the organic extract dried (MgSO₄), filtered and evaporated in vacuo. Elution with hexane through a silica pad yielded the title compound as a white solid.

### e) (3S)-1-(6-Fluoro-1-benzothien-3-yl)-3-methyl-piperazine

A solution of 3-bromo-6-fluoro-1-benzothiophene (1.91 g, 8.3 mmol) in toluene (60 mL) was purged with dry nitrogen, then (2S)-methylpiperazine (1 g, 9.9 mmol), sodium tert-butoxide (1.1 g, 11.6 mmol), tris(dibenzylideneacetone)dipalladium (125 mg) and R-(+)-2,2′-bis(diphenylphosphino)-1,1′-binaphthyl (250 mg) added. After purging the reaction vessel for a further 15 min with dry nitrogen, the reaction mixture was heated at 90° C. for 2 h. Ethyl acetate (250 mL) was added and the mixture washed with ammonia solution (250 mL), then water. The organic extracts were dried (MgSO₄), filtered and evaporated in vacuo. The crude product was purified by flash chromatography on silica, eluting with ethyl acetate, then methanol/dichloromethane (10:90), to yield the title compound.

### f) 1-{2-[(2S)-4-(6-Fluoro-1-benzothien-3-yl)-2-methyl-piperazinyl)ethyl}-3,4-dihydro-1H-2-benzopyran-6-carboxamide

Prepared from (2S)-4-(6-fluoro-1-benzothien-3-yl)-2-methylpiperazine and 2-[6-(aminocarbonyl)-3,4-dihydro-1H-2-benzopyran-1-yl]ethyl methanesulfonate as described for Example 1c), to yield the title compound. M+1=454.

### EXAMPLE 14

### 1-{2-[(2R)-4-(6-Fluoro-1-benzothien-3-yl)-2-methylpiperazinyl]ethyl}-3,4-dihydro-1H-2-benzopyran-6-carboxamide

Prepared according to the method described for Example 13, replacing (2S)-methylpiperazine by the (2R)-enantiomer in Example 13e), to yield the title compound. M+1=454.

### EXAMPLE 15

### 3-{1-[2-(6-Bromo-3,4-dihydro-1H-2-benzopyran-1-yl)ethyl]-1,2,3,6-tetrahydro-4-pyridinyl}-6-fluoro-1H-indole

A mixture of 6-fluoro-3-(1,2,3,6-tetrahydro-4-pyridinyl)-1H-indole (Q.43 g, 2 mmol), 2-[6-bromo-3,4-dihydro-1H-2-benzopyran-1-yl]ethyl methanesulfonate (0.67 g, 2 mmol), potassium carbonate (0.6 g, 4.3 mmol) and potassium iodide (0.033 g, 0.2 mmol) in acetonitrile (25 mL) was heated at reflux for 1 day with stirring under nitrogen. After cooling to room temperature, water (60 mL) was added and the product extracted into diethyl ether. On washing the combined organic extracts with water, the resultant solid was was filtered off to give the product as a yellow powder (mp 184.0-185.6° C.). M+H=455/7.

### EXAMPLE 16

### 6-Fluoro-3-(1-{2-[6-(1-methyl-1H-1,2,4-triazol-3-yl)-3,4-dihydro-1H-2-benzopyran-1-yl]ethyl}-1,2,3,6-tetrahydro-4-pyridinyl)-1H-indole

### a) 1-(2-{[tert-Butyl(dimethyl)silyl]oxy}ethyl)-N-[(Z)-(dimethylamino)methylidene]-3,4-dihydro-1H-2-benzopyran-6-carboxamide

A mixture of 1-(2-{[tert-butyl(dimethyl)silyl]oxy}ethyl)-3,4-dihydro-1H-2-benzopyran-6-carboxamide (2.8 g, 8.5 mmol), N,N-dimethylformamide dimethylacetal (2.43 g, 20.5 mmol) and toluene (15 ml) was heated under nitrogen at 90° C. for 2 h. The solution was evaporated to dryness to give the title compound as an oil.

### b) 2-[6-(1-Methyl-1H-1,2,4-triazol-3-yl)-3,4-dihydro-1H-2-benzopyran-1-yl]ethanol

A solution of 1-(2-{[tert-butyl(dimethyl)silyl]oxy}ethyl)-N-[(Z)-(dimethylamino)methylidene]-3,4-dihydro-1H-2-benzopyran-6-carboxamide (1.2 g, 3.08 mmol)) and methylhydrazine (0.28 g, 6.2 mmol) in methanol (5 mL) was stirred under nitrogen at room temperature for 3 h. Acetic acid (5 mL) and water (1 mL) were added and the reaction stirred overnight. The mixture was evaporated to dryness, saturated aqueous sodium carbonate solution added and the product extracted into dichloromethane. The combined organic extracts were washed (brine), dried (MgSO₄) and evaporated in vacuo. The crude product was purified by flash chromatography on silica, eluting with methanol/ethyl acetate (0:100 to 10:90), to give the title compound as an oil.

### c) 2-[6-(1-Methyl-1H-1,2,4-triazol-3-yl)-3,4-dihydro-1H-2-benzopyran-1-yl)ethyl methanesulfonate was prepared from 2-[6-(1-methyl-1H-1,2,4-triazol-3-yl)-3,4-dihydro-1H-2-benzopyran-1-yl]ethanol using the method described in Example 1b)

### d) 6-Fluoro-3-(1-{2-[6-(1-methyl-1H-1,2,4-triazol-3-yl)-3,4-dihydro-1H-2-benzopyran-1-yl]ethyl}-1,2,3,6-tetrahydro-4-pyridinyl)-1H-indole was prepared from 2-[6-(1-methyl-1H-1,2,4-triazol-3-yl)-3,4-dihydro-1H-2-benzopyran-1-yl]ethyl methanesulfonate, using the method described in Example 1c)

### EXAMPLE 17

### 1-[2-(4-(6-Fluoro-1H-indol-3-yl)-3,6-dihydro-1(2H)-pyridinyl)ethyl]-N,N-dimethyl-3,4-dihydro-1H-2-benzopyran-6-carboxamide

### a) 1-(2-{[tert-Butyl(dimethyl)silyl]oxy}ethyl)-N,N-dimethyl-3,4-dihydro-1H-2-benzopyran-6-carboxamide

Ethyl chloroformate (0.26 g, 2.4 mmol) was added under nitrogen to a solution of 1-(2-{[tert-butyl(dimethyl)silyl]oxy}ethyl)-3,4-dihydro-1H-2-benzopyran-6-carboxylic acid (0.77 g, 2.29 mmol) and triethylamine (0.7 g, 6.8 mmol) in dichloromethane (15 mL), maintaining the temperature at 0° C. After 1 h at 0° C., the solution was stirred at room temperature for 15 min, then recooled to 0° C. A 2M solution of dimethylamine in tetrahydrofuran (5 mL, 10 mmol) was added and the mixture stirred at room temperature for 3 days. Water was added and the product extracted into dichloromethane. The combined organic extracts were washed (brine), dried (MgSO4) and evaporated in vacuo to give the product as an oil.

### b) 2-[6-(N,N-Dimethylaminocarbonyl)-3,4-dihydro-1H-2-benzopyran-1-yl]ethanol

The title compound was prepared from 1-(2-{[tert-butyl(dimethyl)silyl)oxy}ethyl)-N,N-dimethyl-3,4-dihydro-1H-2-benzopyran-6-carboxamide as described for Example 1a).

### c) 2-[6-(N,N-Dimethylaminocarbonyl)-3,4-dihydro-1H-2-benzopyran-1-yl]ethyl methanesulfonate

The title compound was prepared from 2-[6-(N,N-dimethylaminocarbonyl)-3,4-dihydro-1H-2-benzopyran-1-yl]ethanol as described for Example 1b).

### d) 1-[2-(4-(6-Fluoro-1H-indol-3-yl)-3,6-dihydro-1(2H)-pyridinyl)ethyl]-N,N-dimethyl-3,4-dihydro-1H-2-benzopyran-6-carboxamide

A mixture of 6-fluoro-3-(1,2,3,6-tetrahydro-4-pyridinyl)-1H-indole (0.2 g, 0.93 mmol), 2-[6-(N,N-dimethylaminocarbonyl)-3,4-dihydro-1H-2-benzopyran-1-yl]ethylmethanesulfonate (0.27 g, 0.83 mmol), potassium carbonate (0.25 g, 1.8 mmol) and potassium iodide (0.013 g, 0.08 mmol) in acetonitrile (10 mL) was refluxed for 2 days with stirring under nitrogen. After cooling to room temperature, water (15 mL) was added and the product extracted into ethyl acetate. The combined organic extracts were washed with water, dried (MgSO₄) and evaporated in vacuo. The crude product was purified by flash chromatography on silica, eluting with methanol/ethyl acetate (0:100 to 20:80), to give the title compound as an oil. M+H=448.

The following Examples were made by substituting the 2-[6-(N,N-dimethylaminocarbonyl)-3,4-dihydro-1H-2-benzopyran-1-yl]ethyl methanesulfonate in the above example with alternative methanesulfonate derivatives:

### EXAMPLE 18

### 1-[2-(4-(6-Fluoro-1H-indol-3-yl)-3,6-dihydro-1(2H)-pyridinyl)ethyl]-N-propyl-3,4-dihydro-1H-2-benzopyran-6-carboxamide

Prepared from 2[6-(N-propylaminocarbonyl)-3,4-dihydro-1H-2-benzopyran-1-yl]ethyl methanesulfonate. M+H=462.

### EXAMPLE 19

### 6-Fluoro-3-(1-{2-[6-(methylsulfonyl)-3,4-dihydro-1H-2-benzopyran-1-yl]ethyl}-1,2,3,6-tetrahydro-4-pyridinyl)-1H-indole

Prepared from 2-[6-methylsulfonyl-3,4-dihydro-1H-2-benzopyran-1-yl]ethyl methanesulfonate. M+H=455.

### EXAMPLE 20

### 1-[2-(4-(6-Fluoro-1H-indol-3-yl)-3,6-dihydro-1(2H)-pyridinyl)ethyl]-N-methyl-3,4-dihydro-1H-2-benzopyran-6-carboxamide

Prepared from 2-[6-(N-methylaminocarbonyl)-3,4-dihydro-1H-2-benzopyran-1-yl]ethyl methanesulfonate. M+H=434.

### EXAMPLE 21

### 6-Fluoro-3-(1-{2-[6-(4-morpholinylcarbonyl)-3,4-dihydro-1H-2-benzopyran-1-yl]ethyl}-1,2,3,6-tetrahydro-4-pyridinyl)-6-fluoro-1H-indole

Prepared from 2-[6-(4-morpholinylcarbonyl)-3,4-dihydro-1H-2-benzopyran-1-yl]ethyl methanesulfonate. M+H=490.

Mixtures of racemates or diastereoisomers were separated by preparative HPLC using an appropiate chiral column (e.g. Chiralpak-AD or Chiralcel-OJ) and solvent system (e.g. mixtures of hexane, ethyl acetate and diethylamine) to produce examples of single enantiomers:

### EXAMPLE 22

### (1S)-1-[2-(4-(6-Fluoro-1H-indol-3-yl)-3,6-dihydro-1(2H)-pyridinyl)ethyl]-3,4-dihydro-1H-2-benzopyran-6-carboxamide

M+H=420.

### EXAMPLE 23

### (1S)-1-[2-(4-(6-Fluoro-1H-indol-3-yl)-3,6-dihydro-1(2H)-pyridinyl)ethyl]-N-methyl-3,4-dihydro-1H-2-benzopyran-6-carboxamide

M+H=450.

### EXAMPLE 24

### (1S)-1-{2-[(2R)-2-Methyl-4-(1-naphthyl)piperazinyl]ethyl}-3,4-dihydro-1H-2-benzopyran-6-carboxamide

M+H=450.

### EXAMPLE 25

### (1S)-1-{2-[(2S)-2-Methyl-4-(1-naphthyl)piperazinyl]ethyl}-3,4-dihydro-1H-2-benzopyran-6-carboxamide

M+H=450.

The following Examples were prepared by Rapid Parallel Synthesis (RPS), varying the secondary amine indicated, using the general description as described below:

To 2-[6-(aminocarbonyl)-3,4-dihydro-1H-2-benzopyran-1-yl]ethyl methanesulfonate (13.5 mg, 0.045 mmol) in acetonitrile (0.5 mL) was added potassium iodide (0.5 mg, 0.003 mmol) in acetonitrile (1 mL), solid potassium carbonate (8.28 mg, 0.06 mmol) and an example from a number of selected secondary amines (0.03 mmol) dissolved in acetonitrile (1 mL), and the resultant suspension heated at 80° C. for 3 to 4 days. Each reaction was added to an ‘Isolute SCX’ ion exchange column (1 mL) previously activated with methanol (3 mL) and the column eluted with methanol (6 mL). Each column was then eluted with 2.3M ammonia in methanol (8 mL) and the fractions evaporated to give the required product (average yield 78%). In cases where there was unreacted secondary amine present, the appropriate product was dissolved in chloroform (3 mL) and agitated at room temperature in the presence of methylisocyanate polystyrene resin (100 mg). The resin was filtered off and each solution purified via an exchange column as described above.

### EXAMPLE 26

### 1-{2-[4-(6-Fluoro-1H-indol-3yl-)piperidinyl]ethyl}-3,4-dihydro-1H-2-benzopyran-6-carboxamide

Prepared from 6-fluoro-3-(4-piperidinyl)-1H-indole. M+H=422.

### EXAMPLE 27

### 1-[2-(4-(1-Naphthyl)-3,6-dihydro-1(2H)-pyridinyl)ethyl]-3,4-dihydro-1H-2-benzopyran-6-carboxamide

Prepared from 4-(1-naphthyl)-1,2,3,6-tetrahydropyridine. M+H=413.

### EXAMPLE 28

### 1-[2-(4-(4-Fluoro-1-benzofuran-7-yl)-3,4-dihydro-1(2H)-pyridinyl)ethyl]-3,4-dihydro-1H-2-benzopyran-6-carboxamide

Prepared from 4-(4-fluoro-1-benzofuran-7-yl)-1,2,3,6-tetrahydropyridine. M+H=421.

### EXAMPLE 29

### 1-[2-(4-(1-Benzothieny-7-yl)-3,4-dihydro-1(2H)-pyridinyl)ethyl]-3,4-dihydro-1H-2-benzopyran-6-carboxamide

Prepared from 4-(1-benzothieny-7-yl)-1,2,3,6-tetrahydropyridine. M+H=419.

### EXAMPLE 30

### 1-{2-[(2R,4S)-4-(6-Fluoro-1H-indol-3-yl)-2-methylpiperidinyl]ethyl}-3,4-dihydro-1H-2-benzopyran-6-carboxamide

Prepared from 6-fluoro-3-[(2R,4S)-2-methylpiperidinyl)-1H-indole. M+H=436.

### EXAMPLE 31

### 1-{2-[(2S,4R)-4-(6-Fluoro-1H-indol-3-yl)-2-methylpiperidinyl]ethyl}-3,4-dihydro-1H-2-benzopyran-6-carboxamide

Prepared from 6-fluoro-3-[(2S, 4R)-2-methylpiperidinyl]-1H-indole. M+H=436.

### EXAMPLE 32

### 1-{2-[(2R,4S)-2-Methyl-4-(2-naphthyl)piperidinyl]ethyl}-3,4-dihydro-1H-2-benzopyran-6-carboxamide

Prepared from (2R,4S)-2-methyl-4-(2-naphthyl)piperidine. M+H=429.

### EXAMPLE 33

### 1-{2-[(3R)-3-(6-Fluoro-1H-indol-3-yl)pyrrolidinyl]ethyl}-3,4-dihydro-1H-2-benzopyran-6-carboxamide

Prepared from (3R)-6-fluoro-3-(3-pyrrolidinyl)-1H-indole. M+H=408.

### EXAMPLE 34

### 1-[2-(4-(6-Fluoro-1,2-benzisoxazol-3-yl)-3,6-dihydro-1(2H)-pyridinyl)ethyl]-3,4-dihydro-1H-2-benzopyran-6-carboxamide

Prepared from 6-fluoro-3-(1,2,3,6-tetrahydro-4-pyridinyl)-1,2-benzisoxazole. M+H=422.

### EXAMPLE 35

### 1-[2-(4-(6,7-Difluoro-1H-indol-3yl-)-3,6-dihydro-1(2H)-pyridinyl)ethyl]-3,4-dihydro-1H-2-benzopyran-6-carboxamide

Prepared from 6,7-difluoro-3-(1,2,3,6-tetrahydro-4-pyridinyl)-1H-indole. M+H=438.

### EXAMPLE 36

### 1-{2-[4-(6,7-Difluoro-1H-indol-3-yl)-1-piperidinyl)ethyl}-3,4-dihydro-1H-2-benzopyran-6-carboxamide

Prepared from 6,7-difluoro-3-(4-piperidinyl)-1H-indole. M+H=440.

### EXAMPLE 37

### 1-[2-(4-(6-Fluoro-1-methyl-1H-indazol-3yl-)-3,6-dihydro-1(2H)-pyridinyl)ethyl]-3,4-dihydro-1H-2-benzopyran-6-carboxamide

Prepared from 6-fluoro-1-methyl-3-(1,2,3,6-tetrahydro-4-pyridinyl)-1H-indazole. M+H=435.

### EXAMPLE 38

### 1-[2-(4-(6-Fluoro-1H-indazol-3yl-)-3,6-dihydro-1(2H)-pyridinyl)ethyl]-3,4-dihydro-1H-2-benzopyran-6-carboxamide

Prepared from 6-fluoro-1-3-(1,2,3,6-tetrahydro-4-pyridinyl)-1H-indazole. M+H=421.

### EXAMPLE 39

### 1-[2-(4-(6-Fluoro-1-benzofuran-3-yl)-3,4-dihydro-1(2H)-pyridinyl)ethyl]-3,4-dihydro-1H-2-benzopyran-6-carboxamide

Prepared from 4-(6-fluoro-1-benzofuran-3-yl)-1,2,3,6-tetrahydropyridine. M+H=421.

### EXAMPLE 40

### 1-[2-(4-(6,7-Difluoro-1-benzofuran-3-yl)-3,4-dihydro-1(2H)-pyridinyl)ethyl]-3,4-dihydro-1H-2-benzopyran-6-carboxamide

Prepared from 4-(6,7-difluoro-1-benzofuran-3-yl)-1,2,3,6-tetrahydropyridine. M+H=439.

### EXAMPLE 41

### 1-[2-(4-(7-Fluoro-1H-indol-3yl-)-3,6-dihydro-1(2H)-pyridinyl)ethyl]-3,4-dihydro-1H-2-benzopyran-6-carboxamide

Prepared from 7-fluoro-3-(1,2,3,6-tetrahydro-4-pyridinyl)-1H-indole. M+H=420.

### EXAMPLE 42

### 1-{2-[4-(7-Fluoro-1H-indol-3yl-)piperidinyl]ethyl}-3,4-dihydro-1H-2-benzopyran-6-carboxamide

Prepared from 7-fluoro-3-(4-piperidinyl)-1H-indole. M+H=422.

### EXAMPLE 43

### 1-{2-[4-(6-Fluoro-1-benzofuran-3-yl)piperidinyl]ethyl}-3,4-dihydro-1H-2-benzopyran-6-carboxamide

Prepared from 4-(6-fluoro-1-benzofuran-3-yl)piperidine. M+H=423.

### EXAMPLE 44

### 1-[2-(4-(7-Fluoro-1-naphthyl)-3,6-dihydro-1(2H)-pyridinyl)ethyl]-3,4-dihydro-1H-2-benzopyran-6-carboxamide

Prepared from 4-(7-fluoro-1-naphthyl)-1,2,3,6-tetrahydropyridine. M+H=431.

### EXAMPLE 45

### 1-[2-(4-(6-Fluoro-1-methyl-1H-indol-3yl-)-3,6-dihydro-1(2H)-pyridinyl)ethyl]-3,4-dihydro-1H-2-benzopyran-6-carboxamide

Prepared from 6-fluoro-1-methyl-3-(1,2,3,6-tetrahydro-4-pyridinyl)-1H-indole. M+H=434.

### EXAMPLE 46

### 1-[2-(4-(5-Methoxy-1H-indol-3yl-)-3,6-dihydro-1(2H) -pyridinyl)ethyl]-3,4-dihydro-1H-2-benzopyran-6-carboxamide

Prepared from 5-methoxy-3-(1,2,3,6-tetrahydro-4-pyridinyl)-1H-indole. M+H=432.

### EXAMPLE 47

### 1-{2-[4-(1-Naphthyl)piperidinyl]ethyl}-3,4-dihydro-1H-2-benzopyran-6-carboxamide

Prepared from 4-(1-naphthyl)piperidine. M+H=415.

### EXAMPLE 48

### 1-{2-[(2R)-4-(6-Fluoro-1-naphthyl)-2-methylpiperazinyl]ethyl}-1,3-dihydro-2-benzofuran-5-carboxamide

### a) 5-Cyano-1,3-dihydro-2-benzofuran-1-yl acetate

To a stirred solution of 1-oxo-1,3-dihydro-2-benzofuran-5-carbonitrile (1.0 g, 6.28 mmol) in dry dichloromethane (50 mL) at −78° C. under nitrogen was added 1M DIBAL-H in toluene (7.54 mL, 7.54 mmol). After stirring for 4 h 4-dimethylaminopyridine (850 mg, 6.9 mmol) and pyridine (1.52 mL, 18.9 mmol) were added; acetic anhydride (2.36 mL, 25.1 mmol) was then added dropwise and the reaction stirred at −78° C. for 12 h. The reaction mixture was neutralised with aqueous ammonium chloride and extracted with dichloromethane, dried (MgSO₄), filtered and the solvent removed in vacuo. The title compound was obtained as a yellow solid.

### b) Ethyl (5-cyano-1,3-dihydro-2-benzofuran-1-yl)acetate

A suspension of ethyl bromoacetate (1.07 mL, 9.72 mmol), zinc (950 mg, 14.6 mmol) and iodine (422 mg, 1.62 mmol) in dioxane (50 mL) was sonicated under nitrogen for 50 min. The solvent was removed in vacuo and the residue dissolved in dry dichloromethane (50 mL). The slurry was cooled to −78° C. and a solution of 5-cyano-1,3-dihydro-2-benzofuran-1-yl acetate (659 mg, 3.24 mmol) in dichloromethane (5 mL) was added. Trimethylsilyl trifluoromethanesulfonate (1.2 mL, 6.48 mmol) was then added dropwise and the mixture stirred at −78° C. for 1 h. The reaction mixture was quenched with aqueous ammonium chloride and extracted into dichloromethane. The organic layer was dried (MgSO₄) and the solvent removed in vacuo to give the title compound.

### c) (5-Cyano-1,3-dihydro-2-benzofuran-1-yl)acetic acid

A solution of ethyl (5-cyano-1,3-dihydro-2-benzofuran-1-yl)acetate (225 mg, 0.973 mmol) and aqueous lithium hydroxide (2.5M) (0.920 mL, 2.3 mmol) in tetrahydrofuran (28 mL) was stirred at room temperature. After 4 h the mixture was diluted with 1M sodium hydroxide and washed with diethylether. The organic layer was acidified with 1N hydrochloric acid, extracted with ethyl acetate and dried (MgSO₄). The solvent was removed in vacuo to give the corresponding acid as a white solid.

### d) 1-(2-Hydroxyethyl)-1,3-dihydro-2-benzofuran-5-carbo-nitrile

To a solution of (5-cyano-1,3-dihydro-2-benzofuran-1-yl)acetic acid (187 mg, 0.92 mmol) in tetrahydrofuran (8 mL) at 0° C. under nitrogen was added diisopropylethylamine (0.321 mL, 1.84 mmol) and ethyl chloroformate (0.105 mL, 1.10 mmol) and stirred at 0° C. After 3 h, a solution of sodium borohydride (119 mg, 3.13 mmol) in water (7 mL) was added and stirred for 15 min. The reaction was quenched by addition of aqueous ammonium chloride and the residue extracted with ethyl acetate. Evaporation of the organic extracts gave the title compound.

### e) 1-(2-Hydroxyethyl)-1,3-dihydro-2-benzofuran-5-carbox-amide

To a stirred solution of 1-(2-hydroxyethyl)-1,3-dihydro-2-benzofuran-5-carbonitrile (192 mg, 1.01 mmol) in dichloromethane (2.5 mL) cooled at 0° C. was added 33% hydrogen peroxide (0.522 mL, 5.07 mmol), tetrabutylammonium hydrogen sulfate (86 mg, 0.25 mmol) and 2N sodium hydroxide (1 mL, 2.02 mmol). The reaction mixture was kept in an ice-bath for 5 min., then warmed to room temperature. After 1 h, the reaction mixture was diluted in dichloromethane and washed twice with brine. The aquous layer was acidified with 1N hydrochloric acid and extracted with an ethyl acetate:methanol mixture. The organic layer was dried (MgSO₄) and the solvents removed in vacuo to give the title compound.

### f) 1-{2-[(2R)-4-(6-Fluoro-1-naphthyl)-2-methylpiperazinyl]ethyl}-1,3-dihydro-2-benzofuran-5-carboxamide

The title compound was prepared from 1-(2-hydroxyethyl)-1,3-dihydro-2-benzofuran-5-carboxamide by initial formation of the methanesulfonate as described for Example 1b), and condensation of this sulfonate with (2R)-4-(6-fluoro-1-naphthyl)-2-methylpiperazine as described for Example 1c). M+1=434.

### EXAMPLE 49

### 5-[2-(4-(6-Fluoro-1H-indolyl-3-yl)-3,6-dihydro-1(2H)-pyridinyl)ethyl]-5,6,7,8-tetrahydro-2-naphthalenecarboxamide, maleate

### a) Ethyl (6-cyano-3,4-dihydro-1(2H)-naphthalenylidene)ethanoate

To a stirred solution of triethyl phosphonoacetate (4.39 mL, 22.5 mmol) in dry tetrahydrofuran was added sodium hydride (60% dispersion in oil) (0.94 g, 23.5 mmol) over 5 min. The solution was cooled in ice-water and 6-cyano-1-tetralone (3.21 g, 18.8 mmol) in tetrahydrofuran (30 mL) added over 5 min., then allowed to warm to room temperature and stirred overnight. The reaction mixture was quenched with water and extracted with ethyl acetate. The combined-organic extracts were washed with brine, dried (MgSO₄), filtered and evaporated in vacuo. The crude product was purified by flash chromatography on silica, eluting with ethyl acetate/hexane (20:80 to 30:70), to yield the title compound as a mixture of the E and Z isomers.

### b) Ethyl (6-cyano-1,2,3,4-tetrahydro-1-naphthalenyl)ethanoate

To a solution of ethyl (6-cyano-3,4-dihydro-1(2H)-naphthalenylidene)ethanoate (0.5 g, 2.1 mmol) in methanol (50 mL) was added 5% palladium on charcoal (0.05 g) and the reaction mixture shaken under an atmosphere of hydrogen (25 psi) in a Parr apparatus until tlc showed complete reaction of starting material. The catalyst was filtered off through a bed of Celite and the solvent removed in vacuo. The crude product was purified by flash chromatography on silica, eluting with diethyl ether/hexane (20:80) to yield the title compound as a white solid.

### c) 2-(6-Cyano-1,2,3,4-tetrahydro-1-naphthalenyl)ethanol

To a stirred solution of ethyl (6-cyano-1,2,3,4-tetrahydro-1-naphthalenyl)acetate (0.65 g, 2.67 mmol) in tetrahydrofuran (30 mL) was added lithium borohydride (0.118 g, 5.3 mmol), and the reaction mixture heated at reflux for 6 h. The solution was cooled, diluted with ethyl acetate and washed with water, then brine. The organic extracts were dried (MgSO₄), filtered and evaporated in vacuo. The crude product was purified by flash chromatography on silica, eluting with ethyl acetate/hexane (25:75 to 50:50), to yield the title compound as a white solid.

### d) 5-[2-(4-(6-Fluoro-1H-indolyl-3-yl) -3,6-dihydro-1(2H) -pyridinyl)ethyl]-5,6,7,8-tetrahydro-2-naphthalenecarbonitrile

To a stirred solution of 2-(6-cyano-1,2,3,4-tetrahydro-1-naphthalenyl)ethanol (0.19 g, 0.95 mmol) in dichloromethane (10 mL) and triethylamine (0.158 mL, 1.13 mmol) at 0° C. was added methanesulfonyl chloride (0.088 mL, 1.13 mmol). After stirring for 2 h at 0° C., the reaction mixture was diluted with ethyl acetate and washed with water, then brine. The organic extracts were dried (MgSO₄), filtered and evaporated in vacuo.

To a solution of the crude methanesulfonate in acetonitrile (8 mL) was added 6-fluoro-3-(1,2,3,6-tetrahydropyridin-4-yl)-1H-indole (0.31 g, 1.43 mmol), potassium carbonate (0.20 g, 1.43 mmol) and potassium iodide (10 mg). The reaction mixture was heated at reflux for 18 h, cooled and extracted from water into ethyl acetate. The combined organic extracts were washed with brine, dried (MgSO₄), filtered and evaporated in vacuo. The crude product was purified by flash chromatography on silica, eluting with ethyl acetate, to yield the title compound as a gum.

### e) 5-[2-(4-(6-Fluoro-7H-indolyl-3-yl)-3,6-dihydro-1(2H)-pyridinyl)ethyl]-5,6,7,8-tetrahydro-2-naphthalenecarboxamide, maleate

5-[2-(4-(6-Fluoro-7H-indolyl-3-yl)-3,6-dihydro-1(2H)-pyridinyl)ethyl]-5,6,7,8-tetrahydro-2-naphthalenecarbonitrile (0.1 g, 0.25 mmol) in boron trifluoride.acetic acid complex (2 mL) and water (0.045 mL, 2.5 mmol) was heated at 80° C. for 1 h. The reaction mixture was cooled, diluted with ethyl acetate and washed with 2M sodium hydroxide (aq), then with brine. The organic extract was dried (MgSO₄), filtered and evaporated in vacuo. The crude product was purified by flash chromatography on silica, eluting with ethyl acetate, then methanol/chloroform (5:95 to 20:80), to yield the title compound as its free base. This was dissolved in methanol and maleic acid (1 equiv.) in methanol added. The solvent was removed in vacuo and the resultant gum triturated with diethyl ether, then ethyl acetate, to yield the title compound as a yellow solid. M+H=418.

### EXAMPLE 50

### 5-[2-(4-(6-Fluoro-1H-indolyl-3-yl)-3,6-dihydro-1(2H)-pyridinyl)ethyl]-7,8-dihydro-2-naphthalenecarboxamide

### a) Ethyl (6-cyano-3,4-dihydro-1-naphthalenyl)ethanoate

To a solution of ethyl (2E/Z)-(6-cyano-3,4-dihydro-1(2H)-naphthalenylidene)ethanoate (0.66 g, 2.74 mmol) in tetrahydrofuran (25 mL) and ethanol (0.25 mL) was added sodium hydride (60% dispersion in oil) (0.11 g, 2.74 mmol), and the reaction mixture heated at reflux for 1.5 h. The reaction was cooled, acidified with acetic acid and diluted with ethyl acetate. The solution was washed with saturated sodium bicarbonate (aq), dried (MgSO₄), filtered and evaporated in vacuo. The crude product was purified by flash chromatography on silica, eluting with ethyl acetate/hexane (0:100 to 20:80), to yield the title compound as a white solid.

### b) 2-(6-cyano-3,4-dihydro-1-naphthalenyl)ethanol

To a stirred solution of ethyl (6-cyano-3,4-tetrahydro-1-naphthalenyl) ethanoate (0.185 g, 0.77 mmol) in tetrahydrofuran (8 mL) was added lithium borohydride (0.034 g, 1.54 mmol), and the reaction mixture heated at reflux for 4 h. The solution was cooled, diluted with ethyl acetate and washed with water, then brine. The organic extracts were dried (MgSO₄), filtered and evaporated in vacuo. The crude product was purified by flash chromatography on silica, eluting with ethyl acetate/hexane (20:80 to 50:50), to yield the title compound as a white solid.

### c) 5-[2-(4-(6-fluoro-1H-indolyl-3-yl)-3,6-dihydro-1(2H)-pyridinyl)ethyl]-7,8-dihydro-2-naphthalenecarbonitrile

To a stirred solution of 2-(6-cyano-3,4-tetrahydro-1-naphthalenyl)ethanol (0.15 g, 0.75 mmol) in dichloromethane (12 mL) and triethylamine (0.115 mL, 1.12 mmol) at 0° C. was added methanesulfonyl chloride (0.064 mL, 0.82 mmol). After stirring for 2 h at 0° C., the reaction mixture was diluted with ethyl acetate and washed with water, then brine. The organic extracts were dried (MgSO₄), filtered and evaporated in vacuo.

To a solution of the crude methanesulfonate in acetonitrile (8 mL) was added 6-fluoro-3-(1,2,3,6-tetrahydropyridin-4-yl)-1H-indole (0.24 g, 1.12 mmol), potassium carbonate (0.15 g, 1.12 mmol) and potassium iodide (20 mg). The reaction mixture was heated at reflux for 18 h, cooled and extracted from water into ethyl acetate. The combined organic extracts were washed with brine, dried (MgSO₄), filtered and evaporated in vacuo. The crude product was purified by flash chromatography on silica, eluting with ethyl acetate then methanol/chloroform (10:90), to yield the title compound as a gum.

### d) 5-[2-(4-(6-Fluoro-1H-indolyl-3-yl)-3,6-dihydro-1(2H) -pyridinyl)ethyl]-7,8-dihydro-2-naphthalenecarboxamide

5-[2-(4-(6-fluoro-1H-indolyl-3-yl-)-3,6-dihydro-1(2H) -pyridinyl)ethyl]-7,8-dihydro-2-naphthalenecarbonitrile (0.165 g, 0.42 mmol) in boron trifluoride.acetic acid complex (3 mL) and water (0.075 mL, 4.2 mmol) was heated at 80° C. for 1.5 h. The reaction mixture was cooled, diluted with ethyl acetate and washed with 5M sodium hydroxide (aq), then with brine. The organic extract was dried (MgSO₄), filtered and evaporated in vacuo. The crude product was purified by flash chromatography on silica, eluting with methanol/chloroform (0:100 to 20:80), to yield the title compound as a yellow gum. M+1=416.

### EXAMPLE 51

### N-{8-[2-(4-(6-fluoro-1H-indolyl-3-yl)-3,6-dihydro-1(2H) -pyridinyl)ethyl]-5,6,7,8-tetrahydro-2-naphthalenyl}acetamide

### a) Ethyl (2E/Z)-(7-nitro-3,4-dihydro-1(2H) -naphthalenylidene)ethanoate

To a stirred solution of triethyl phosphonoacetate (8.36 mL, 42.9 mmol) in dry THF (200 mL) was added sodium hydride (60% oil dispersion) (1.72 g, 42.9 mmol) in portions over 10 min. After stirring for 30 min, 7-nitro-1-tetralone (7.3 g, 39 mmol) in THF (40 mL) was added rapidly and the solution stirred for a further 20 h. The reaction mixture was diluted with ethyl acetate and washed with water (2×), then brine. The combined organic extracts were dried (MgSO₄), filtered and evaporated in vacuo. The crude product was purified by flash chromatography on silica, eluting with ethyl acetate/hexane (0:100 to 10:90), to yield the title compound as a mixture of geometric isomers.

### b) Ethyl (7-amino-1,2,3,4-tetrahydro-1-naphthalenyl) ethanoate

A mixture of ethyl (2E/Z)-(7-nitro-3,4-dihydro-1(2H)-naphthalenylidene)ethanoate (0.7 g, 2.7 mmol) and 10% palladium on carbon (0.1 g) in ethanol (50 mL) was hydrogenated at 65 psi for 2 h. The catalyst was filtered off through Celite and the solvent removed in vacuo to yield the title compound as an oil.

### c) Ethyl [7-(acetylamino)-1,2,3,4-tetrahydro-1-naphthalenyl]ethanoate

To a stirred solution of ethyl (7-amino-1,2,3,4-tetrahydro-1-naphthalenyl)ethanoate (0.57 g, 2.5 mmol) in dichloromethane (15 mL) and pyridine (0.4 mL, 5 mmol) was added acetic anhydride (0.28 mL, 3 mmol). The reaction was stirred for 4 days at room temperature, diluted with ethyl acetate and washed with aqueous citric acid, then brine. The organic extracts were dried (MgSO₄), filtered and evaporated in vacuo. The crude product was purified by flash chromatography on silica, eluting with ethyl acetate/hexane (25:75 to 60:40), to yield the title compound.

### d) N-[8-(2-Hydroxyethyl)-5,6,7,8-tetrahydro-2-naphthalenyl]acetamide

To a stirred solution of ethyl [7-(acetylamino)-1,2,3,4-tetrahydro-1-naphthalenyl]ethanoate (0.25 g, 0.92 mmol) in THF (10 mL) was added lithium borohydride (0.041 g, 1.84 mmol), and the reaction heated at reflux for 3 h. the mixture was cooled to room temperature, diluted with ethyl acetate and washed with water, then brine. The organic extracts were dried (MgSO₄), filtered and evaporated in vacuo. The crude product was purified by flash chromatography on silica, eluting with ethyl acetate, then methanol/chloroform (10:90), to yield the title compound.

### e) N-{8-[2-(4-(6-fluoro-1H-indolyl-3-yl)-3,6-dihydro-1(2H)-pyridinyl) ethyl]-5,6,7,8-tetrahydro-2-naphthalenyl}acetamide

A solution of N-[8-(2-hydroxyethyl)-5,6,7,8-tetrahydro-2-naphthalenyl]acetamide (0.1 g, 0.44 mmol) in dichloromethane (8 mL) and triethylamine (0.092 mL, 0.66 mmol) was cooled to 0° C. and stirred as methanesulfonyl chloride (0.037 mL, 0.48 mmol) was added. The reaction mixture was stirred for 2 h at 0° C., diluted with ethyl acetate and washed with water, then brine. The organic extracts were dried (MgSO₄), filtered and evaporated in vacuo.

The crude mesylate thus formed was combined with 6-fluoro-3-(1,2,3,6-tetrahydropyridin-4-yl)-1H-indole (0.142 g, 0.66 mmol), potassium carbonate (0.09 g, 0.66 mmol) and potassium iodide (10 mg) in acetonitrile (5 mL) and the suspension heated at reflux with stirring for 18 h. The reaction mixture was cooled, diluted with ethyl acetate and washed with water. The organic extracts were dried (MgSO₄), filtered and evaporated in vacuo. The crude product was purified by flash chromatography on silica, eluting with ethyl acetate then methanol/chloroform (0:100 to 20:80), to yield the required product as a gum. Crystallisation from diethyl ether/methanol gave N-{8-[2-(4-(6-fluoro-1H-indolyl-3-yl)-3,6-dihydro-1(2H)-pyridinyl)ethyl]-5,6,7,8-tetrahydro-2-naphthalenyl}acetamide. M+1=432.

Preparation

### 1-Allyl-1,2,3,4-tetrahydroquinoline-6-carboxylic acid

Method A

### a) Methyl quinoline-6-carboxylate

To a solution of quinoline-6-carboxylic acid (5 g, 28.9 mmol) in dried DMF (50 mL) at room temperature was added solid carbonyl diimidazole (4.92 g, 30.3 mmol) in a single portion. The solution was stirred at room temperature until gas evolution ceased (approx. 1 h), then solid sodium methoxide (3.2 g, 59.2 mmol) added, and the solution stirred for a further 60 min. The mixture was poured into saturated brine (125 mL) and extracted with ethyl acetate (2×125 mL) and chloroform (125 mL). The combined organic extracts were dried (MgSO₄), filtered, and the solvent removed in vacuo to yield the crude product as a yellow solid. This was purified by flash chromatography on silica, eluting with ethyl acetate/hexane (1:1), to give the title compound as a white solid.

### b) Methyl 1,2,3,4-tetrahydroquinoline-6-carboxylate

To a suspension of 10% Pd/C (5 g) in methanol (200 mL) was added methyl quinoline-6-carboxylate (4 g, 21.4 mmol) and solid ammonium formate (35 g, 555 mmol). The suspension was stirred vigorously and heated under reflux under nitrogen for 2 h. The mixture was allowed to cool to room temperature, then the catalyst removed by filtration and washed with further quantities of methanol (2×100 mL). The solvent was removed in vacuo, and the residue dissolved in water (250 mL), which was then saturated with solid sodium hydrogen carbonate. The aqueous solution was extracted with ethyl acetate (3×250 mL), the combined organic extracts dried (MgSO₄), filtered, and the solvent removed in vacuo to yield the title compound as a white solid, suitable for further use without purification.

### c) Methyl 1-allyl-1,2,3,4-tetrahydroquinoline-6-carboxylate

To a solution of methyl 1,2,3,4-tetrahydroquinoline-6-carboxylate (3.88 g, 20.3 mmol) in dried DMF (100 mL) at −10° C. was added sodium hydride (2.5 g 60% dispersion, 62.5 mmol). The suspension was stirred at −10° C. until gas evolution ceased (approx. 1 h), then treated with allyl bromide (5 mL, 57.8 mmol) and allowed to warm to room temperature overnight whilst stirring. The suspension was poured into ice water (1000 mL), and the aqueous mixture extracted with ether (3×250 mL). The combined organic extracts were dried (MgSO₄), filtered, and the solvent removed in vacuo to yield the title compound as an oil which was used without purification.

### d) 1-Allyl-1,2,3,4-tetrahydroquinoline-6-carboxylic acid

To a solution of methyl 1-allyl-1,2,3,4-tetrahydroquinoline-6-carboxylate (5.16 g, 20 mmol) in tetrahydrofuran (100 mL) was added a solution of lithium hydroxide hydrate (2 g, 47.7 mmol) in water (100 mL). The well-stirred mixture was heated under reflux under nitrogen for 18 h, then ethanol (25 mL) and further lithium hydroxide hydrate (2 g, 47.7 mmol) added. The mixture was heated under reflux under nitrogen for a further 24 h, then cooled to room temperature and filtered. The organic solvents were removed in vacuo, and the resulting aqueous solution washed with ether (2×100 mL). It was then acidified to pH 3 by addition of solid citric acid. The resulting precipitate was removed by filtration, washed with water, then dried in vacuo at 60° C. to yield the title compound as an off-white solid.

Method B

### a) 1,2,3,4-Tetrahydroquinoline-6-carboxylic acid

To a suspension of 10% Pd/C (10 g) in methanol (300 mL) was added quinoline-6-carboxylic acid (5.2 g, 30 mmol) and solid ammonium formate (50 g, 793 mmol). The suspension was stirred vigorously and heated under reflux under nitrogen for 2 h. The mixture was allowed to cool to room temperature, then the catalyst removed by filtration and washed with further quantities of methanol (2×250 mL). The solvent was removed in vacuo and the residue dissolved in water (500 mL), which was then saturated with solid sodium hydrogen carbonate. The aqueous solution was extracted with ethyl acetate (4×250 mL), the combined organic extracts dried (MgSO₄), filtered, and the solvent removed in vacuo to yield the title compound as a white solid, suitable for further use without purification.

### b) Allyl 1-Allyl-1,2,3,4-tetrahydroquinoline-6-carboxylate

To a solution of 1,2,3,4-tetrahydroquinoline-6-carboxylic acid (5.3 g, 29.9 mmol) in dried DMF (100 mL) at −10° C. was added sodium hydride (2 g 60% dispersion, 50 mmol). The suspension was stirred at −10° C. until gas evolution ceased (approx. 30 min), then treated with allyl bromide (4 mL, 46.2 mmol) and allowed to warm to room temperature over 2 hours. The suspension was treated with further sodium hydride (2 g 60% dispersion, 50 mmol) and the temperature raised to 65° C. for 2 hours, then treated with further allyl bromide (4 mL, 46.2 mmol). The mixture was stirred at room temperature overnight, then poured into ice water (1000 mL), and the aqueous mixture extracted with ether (3×250 mL) and chloroform (250 mL). The combined organic extracts were dried (MgSO₄), filtered, and the solvent removed in vacuo to yield the title compound as an oil suitable for further use without purification.

### c) 1-Allyl-1,2,3,4-tetrahydroquinoline-6-carboxylic acid

To a solution of allyl 1-allyl-1,2,3,4-tetrahydroquinoline-6-carboxylate (7.7 g, 29.9 mmol) in ethanol (50 mL) was added a solution of lithium hydroxide hydrate (4 g, 95.4 mmol) in water (l50 mL). The well-stirred mixture was heated under reflux under nitrogen for 2.5 h, then cooled to room temperature and filtered. The organic solvents were removed in vacuo, and the resulting aqueous solution washed with ether (2×100 mL). It was then treated with saturated aqueous ammonium chloride (150 mL). The resulting solution was extracted with chloroform (3×50 mL), then acidified to pH 3 by addition of solid citric acid. The resulting dense flocculent precipitate was removed by filtration, washed with water, then dried in vacuo at 60° C. to yield the title compound as an off-white solid.

### EXAMPLE 52

### 1-{2-[4-(6-Fluoro-1H-indol-3-yl)-3,6-dihydro-2H-pyridin-1-yl]ethyl}-N-methyl-1,2,3,4-tetrahydro-quinoline-6-carboxamide

### a) 1-Allyl-N-methyl-1,2,3,4-tetrahydroquinoline-6-carboxamide

To a solution of 1-allyl-1,2,3,4-tetrahydroquinoline-6-carboxylic acid (500 mg, 2.3 mmol) in dried THF (20 mL) was added solid carbonyl diimidazole (800 mg, 4.93 mmol) in a single portion. The solution was stirred at room temperature for 18 h then treated with methylamine in THF (3 mL of 2M), and stirred for a further 4 h. The solvent was removed in vacuo, and the residue dissolved in ethyl acetate (100 mL), washed with water (3×50 mL) then dried (MgSO₄). The drying agent was removed by filtration, and the solvent removed in vacuo. The residue was dissolved in chloroform (100 mL), the solution washed with 0.5M aqueous citric acid (2×50 mL) and dried (K₂CO₃). The drying agent was removed by filtration, and the solvent removed in vacuo to yield the title compound as an off-white solid suitable for further use without purification.

### b) N-Methyl-1-(2-oxoethyl)-1,2,3,4-tetrahydroquinoline-6-carboxamide

To a solution of 1-allyl-N-methyl-1,2,3,4-tetrahydroquinoline-6-carboxamide (400 mg, 1.74 mmol) in THF (20 mL) was added a solution of sodium periodate (750 mg, 3.5 mmol) in water (20 mL). To the well stirred two phase mixture was then added 2 crystals of osmium tetroxide. Stirring was maintained for 2 h at room temperature, then the reaction mixture extracted with chloroform (3×100 mL). The combined organic extracts were dried (MgSO₄), filtered, and the solvent removed in vacuo to yield the title compound as a light tan solid which was used directly for the next step.

### c) 1-{2-[4-(6-Fluoro-1H-indol-3-yl)-3,6-dihydro-2H-pyridin-1-yl]ethyl}-N-methyl-1,2,3,4-tetrahydroquinoline-6-carboxamide

To a solution of N-methyl-1-(2-oxoethyl)-1,2,3,4-tetrahydroquinoline-6-carboxamide (375 mg, 1.61 mmol) in methanol (25 mL) was added 6-fluoro-3-(1,2,3,6-tetrahydropyridin-4-yl)-1H-indole (750 mg, 3.47 mmol) and acetic acid (2 mL). The solution was stirred at room temperature for 2 h then treated with activated, powdered 4 Å molecular sieves and stirred a further 2 h. The solution was then treated with sodium cyanoborohydride (200 mg, 3.18 mmol) and stirred at room temperature overnight. The solids were removed by filtration, the solvent removed in vacuo and the residue treated with aqueous sodium hydroxide (50 mL of 2M). The basic solution was extracted with ethyl acetate (3×100 mL), the combined organic extracts dried (MgSO₄), filtered, and the solvent removed in vacuo to yield the crude title compound as a yellow solid (420 mg, 60%). This was purified by flash chromatography on silica, eluting with ethyl acetate, then a methanol/chloroform gradient (0:100 to 20:80) to give the title compound as a pale yellow solid (mp 121-125° C.). M+1=433.2.

### EXAMPLE 53

### 1-{2-[4-(6-Fluoro-1H-indol-3-yl)-3,6-dihydro-2H-pyridin-1-yl]ethyl}-1,2,3,4-tetrahydroquinoline-6-carboxamide

### a) 1-Allyl-1,2,3,4-tetrahydroquinoline-6-carboxamide

To a solution of 1-allyl-1,2,3,4-tetrahydroquinoline-6-carboxylic acid (1.013 g, 4.66 mmol) in dried THF (40 mL) was added solid carbonyl diimidazole (1.62 mg, 10 mmol) in a single portion. The solution was stirred at room temperature for 18 hours then treated with ammonia in dioxan (30 mL of 0.5M), and stirred a further 24 hours. The solvent was removed in vacuo, and the residue dissolved in ethyl acetate (100 mL). The solution was washed with water (3×50 mL), then dried (MgSO₄), filtered, and the solvent removed in vacuo. The residue was dissolved in chloroform (100 mL), the solution washed with 0.5M aqueous citric acid (2×50 mL) and dried (K₂CO₃). The drying agent was removed by filtration, and the solvent removed in vacuo to yield the title compound as an off-white solid suitable for further use without purification.

### b) 1-(2-Hydroxyethyl)-1,2,3,4-tetrahydroquinoline-6-carboxamide

To a solution of 1-allyl-1,2,3,4-tetrahydroquinoline-6-carboxamide (907 mg, 4.19 mmol) in THF (35 mL) was added a solution of sodium periodate (1.8 g, 8.4 mmol) in water (40 mL). To the well stirred, two phase mixture was then added 2 crystals of osmium tetroxide. Stirring was maintained for 2 h at room temperature, then the reaction mixture was treated with a solution of sodium borohydride (2 g, 52.9 mmol) in ethanol (25 mL). The mixture was stirred at room temperature for 24 h, filtered and extracted with chloroform (3×150 mL). The combined organic extracts were dried (MgSO₄), filtered, and the solvent removed in vacuo to yield the title compound as an off-white solid which was recrystallised from chloroform and ether.

### c) Methanesulfonic acid 2-(6-carbamoyl-3,4-dihydro-2H-quinolin-1-yl)ethyl ester

To a solution of 1-(2-hydroxyethyl)-1,2,3,4-tetrahydroquinoline-6-carboxamide (330 mg, 1.5 mmol) in dried acetonitrile (10 mL) was added triethylamine (1 mL). The solution was cooled to 0° C., then treated with methanesulfonyl chloride (125□L, 1.62 mmol), and maintained at −10° C. overnight. The solution was then treated with further methanesulfonyl chloride (125□L, 1.62 mmol) and stirred at room temperature for 2 h. The solvents were removed in vacuo and the residue dissolved in chloroform (50 mL), washed with saturated aqueous sodium hydrogen carbonate (3×50 mL) and dried (MgSO₄). The drying agent was removed by filtration, and the solvent removed in vacuo to yield the title compound as a pinkish solid which was used directly for the next step.

### d) 1-{2-[4-(6-Fluoro-1H-indol-3-yl)-3,6-dihydro-2H-pyridin-1-yl]ethyl}-1,2,3,4-tetrahydroquinoline-6-carboxamide

To a solution of methanesulfonic acid 2-(6-carbamoyl-3,4-dihydro-2H-quinolin-1-yl)ethyl ester (393 mg, 1.32 mmol) in dried acetonitrile (50 mL) was added potassium iodide (250 mg, 1.5 mmol), potassium carbonate (1 g, 7.2 mmol) and 6-fluoro-3-(1,2,3,6-tetrahydropyridin-4-yl)-1H-indole (650 mg, 3 mmol). The mixture was heated under reflux under nitrogen for 48 h, then cooled to room temperature. The insoluble solids were removed by filtration and the solvent removed in vacuo. The residue was suspended in chloroform (35 mL), filtered and the solvent removed in vacuo. The residue was purified by flash chromatography on silica, eluting with ethyl acetate, then by preparative HPLC eluting with. acetontrile/water/aqueous ammonia (80:20:0.2) on a KR100-5 C18 reverse phase column, to give the title compound as a yellow solid (mp 130-133° C.). M+1=419.2.

### EXAMPLE 54

### 1-{2-[4-(6-Fluoro-1H-indol-3-yl)-3,6-dihydro-2H-pyridin-1-yl]ethyl}-N-(2-hydroxyethyl)-1,2,3,4-tetrahydroquinoline-6-carboxamide

### a) 1-Allyl-N-(2-hydroxyethyl)-1,2,3,4-tetrahydroquinoline-6-carboxamide

To a solution of 1-allyl-1,2,3,4-tetrahydroquinoline-6-carboxylic acid (650 mg, 2.99 mmol) in dried THF (20 mL) was added solid carbonyl diimidazole (1 g, 6.15 mmol) in a single portion. The solution was stirred at room temperature for 24 h then treated with ethanolamine (600□L, 9.94 mmol), and stirred for a further 24 h. The solvent was removed in vacuo, and the residue dissolved in ethyl acetate (100 mL). The solution was washed with water (3×50 mL), dried (MgSO₄), filtered, and the solvent removed in vacuo. The residue was then dissolved in chloroform (100 mL), the solution washed with 0.5M aqueous citric acid (2×50 mL) and dried (K₂CO₃). The drying agent was removed by filtration, and the solvent removed in vacuo to yield the title compound as an off-white solid suitable for further use without purification.

### b) 1-Allyl-N-[2-(tert-butyldimethylsilanyloxy)ethyl]-1,2,3,4-tetrahydroquinoline-6-carboxamide

To a solution of 1-allyl-N-(2-hydroxyethyl)-1,2,3,4-tetrahydroquinoline-6-carboxamide (1.135 g, 4.36 mmol) in dried dichloromethane (50 mL) was added triethylamine (2.5 mL), dimethylaminopyridine (50 mg, 0.41 mmol) and tert-butyldimethylchlorosilane (750 mg, 4.98 mmol). The mixture was stirred at room temperature for 18 h, then washed with aqueous citric acid (2×200 mL of 0.5M) and dried (K₂CO₃). The drying agent was removed by filtration, and the solvent removed in vacuo to yield the title compound as an oil suitable for further use without purification.

### c) 1-(2-Oxo-ethyl)-N-[2-(tert-butyldimethylsilanyloxy)ethyl)-1,2,3,4-tetrahydroquinoline-6-carboxamide

To a solution of 1-allyl-N-[2-(tert-butyldimethylsilanyloxy)ethyl]-1,2,3,4-tetrahydroquinoline-6-carboxamide (1.63 g, 4.35 mmol) in THF (65 mL) was added a solution of sodium periodate (2 g, 9.35 mmol) in water (35 mL). To the well-stirred two phase mixture was then added 2 crystals of osmium tetroxide. Stirring was maintained for 18 h at room temperature, then the reaction mixture extracted with chloroform (3×150 mL). The combined organic extracts were dried (MgSO₄), filtered, and the solvent removed in vacuo to yield the title compound as an off-white solid which was used directly for the next step.

### d) 1-{2-[4-(6-Fluoro-1H-indol-3-yl)-3,6-dihydro-2H-pyridin-1-yl]-ethyl}-1,2,3,4-tetrahydroquinoline-6-carboxylic acid (2-hydroxyethyl)amide

To a solution of 1-(2-oxo-ethyl)-N-[2-(tert-butyldimethylsilanyloxy)ethyl]-1,2,3,4-tetrahydro-quinoline-6-carboxamide (1.6 g, 4.25 mmol) in methanol (50 mL) was added 6-fluoro-3-(1,2,3,6-tetrahydropyridin-4-yl)-1H-indole (1.5 g, 6.94 mmol), acetic acid (2.5 mL) and sodium cyanoborohydride (300 mg, 4.77 mmol) and stirred at room temperature overnight. The solids were then removed by filtration, the solvent removed in vacuo and the residue treated with aqueous sodium hydroxide (1000 mL of 2M). The basic solution was extracted with chloroform (3×100 mL) and ethyl acetate (100 mL), the combined organic extracts dried (MgSO₄), filtered, and the solvent removed in vacuo to yield the crude title compound as a pasty brown solid (1.77 g, 90%). This was purified by flash chromatography on silica, eluting with ethyl acetate, then a methanol/chloroform gradient (0:100 to 10:90) to give the title compound as a yellow solid (300 mg). This was further purified by preparative HPLC eluting with acetontrile/water/aqueous ammonia (70:30:0.2) on a KR100-5 C18 reverse phase column, to give the title compound as a yellow solid (mp 136-140° C.). M+1=463.3

### EXAMPLE 55

### (R)-1-{2-[4-(6-Fluorobenzo[b]thiophen-3-yl)-2-methylpiperazin-1-yl]-ethyl}-1,2,3,4-tetrahydro-quinoline-6-carboxylic acid amide

### a) Methyl (3,4-dihydro-2H-quinolin-1-yl)acetate

To a solution of 1,2,3,4-tetrahydroquinoline (8 g, 60 mmol) in dried acetonitrile (250 mL) were added methyl bromoacetate (6 mL, 63.4 mmol), potassium carbonate (9 g, 65.1 mmol) and potassium iodide (10 g, 60.2 mmol). The mixture was stirred and heated under reflux overnight, cooled to room temperature and the solids removed by filtration. The solvent was removed in vacuo, the residue dissolved in ether (200 mL) and the insoluble solids removed by filtration. The solvent was then removed in vacuo to give the title compound as an almost colourless oil suitable for further use without purification.

### b) 2-(3,4-Dihydro-2H-quinolin-1-yl)ethanol

To a solution of methyl (3,4-dihydro-2H-quinolin-1-yl)acetate (12.39, 59.9 mmol) in dried THF (250 mL) was added lithium borohydride (5 g, 230 mmol). The suspension was stirred at room temperature for 84 h, then the solvent removed in vacuo and the residue treated with water (500 mL). The aqueous solution was extracted with ethyl acetate (3×250 mL) and the combined organic extracts dried (MgSO₄). The drying agent was removed by filtration, and the solvent removed in vacuo to yield the title compound as a colourless oil suitable for further use without purification.

### c) 2-(6-Iodo-3,4-dihydro-2H-quinolin-1-yl)ethanol

To a solution of 2-(3,4-dihydro-2H-quinolin-1-yl)ethanol (5 g, 28.2 mmol) in dried DMF (150 mL) was added N-iodosuccinimide (7 g, 31.1 mmol) in 0.5 g portion over one hour. The solution was stirred at room temperature overnight, then poured into water (1500 mL) and the aqueous solution extracted with ether (3×250 mL). The combined organic extracts were dried (MgSO₄), filtered and the solvent removed in vacuo to yield the crude title compound as a yellow oil. This was purified by flash chromatography on silica eluting with an ethyl acetate/hexane gradient (0:100 to 30:70) to give the title compound as a pale pink oil.

### d) 1-(2-Hydroxyethyl)-1,2,3,4-tetrahydroquinoline-6-carbonitrile

To a solution of 2-(6-iodo-3,4-dihydro-2H-quinolin-1-yl)ethanol (2.44 g, 8.05 mmol) in dried acetonitrile (75 mL) was added sodium cyanide (800 mg, 16.32 mmol), copper iodide (165 mg, 0.87 mmol and tetrakis-triphenylphosphine palladium (0). The mixture was thoroughly purged and degassed with nitrogen then heated under reflux for 2 h. The solution was then cooled to room temperature, diluted with ethyl acetate (250 mL) and the insoluble solids removed by filtration. Solvent was removed in vacuo from the filtrate, and the residue purified by flash chromatography on silica, eluting with an ethyl acetate/hexane gradient 10:90 to 50:50), to give the title compound as a colourless oil.

### e) Methanesulfonic acid 2-(6-cyano-3,4-dihydro-2H-quinolin-1-yl)-ethyl ester

To a solution of 1-(2-hydroxyethyl)-l,2,3,4-tetrahydroquinoline-6-carbonitrile (1.352 g, 6.68 mmol) in dried chloroform (50 mL) was added triethylamine (1.86 mL, 13.3 mmol) and methanesulfonyl chloride (2.309 g, 20.2 mmol). The solution was stirred at room temperature for 24 h then poured into water (250 mL). The aqueous solution was extracted with chloroform (3×50 mL), the combined organic extracts dried (MgSO₄), filtered and the solvent removed in vacuo to yield the crude title compound as a brown oil. This was purified by flash chromatography on silica, eluting with an ethyl acetate/hexane gradient (20:100 to 80:20) to give the title compound as a light brown oil.

### f) (R)-1-{2-[4-(6-Fluorobenzo[b]thiophen-3-yl)-2-methyl-piperazin-1-yl)ethyl}-1,2,3,4-tetrahydro-quinoline-6-carbonitrile

To a solution of methanesulfonic acid 2-(6-cyano-3,4-dihydro-2H-quinolin-1-yl)ethyl ester (148 mg, 0.53 mmol) in dried acetonitrile (10 mL) was added potassium iodide (88 mg, 0.53 mmol), potassium carbonate (146 mg, 1.06 mmol) and (R)-[4-(6-fluorobenzo[b]thiophen-3-yl)-2-methylpiperazine (120 mg, 0.48 mmol). The solution was stirred under reflux under nitrogen for 36 h, then poured into water (250 mL). The aqueous solution was extracted with ethyl acetate (2×125 mL) and the combined organic extracts dried (MgSO₄). The drying agent removed by filtration and the solvent removed in vacuo to yield the crude title compound as a brown oil. This was purified by flash chromatography on silica, eluting with ethyl acetate/hexane (50:50) to give the title compound as a pale yellow oil.

### g) (R)-1-{2-[4-(6-Fluorobenzo[b]thiophen-3-yl)-2-methylpiperazin-1-yl]ethyl}-1,2,3,4-tetrahydro-quinoline-6-carboxamide

To a solution of (R)-1-{2-[4-(6-fluorobenzo[b]thiophen-3-yl)-2-methyl-piperazin-1-yl]ethyl}-1,2,3,4-tetrahydroquinoline-6-carbonitrile (59 mg, 0.13 mmol) in boron trifluoride-acetic acid complex (2 mL) was added water (15□L). The solution was heated in an air bath for 2 minutes, until effervescence was observed, then cooled to room temperature and poured into water (50 mL). The water was extracted with ethyl acetate (3×25 mL), the combined organic extracts were washed with aqueous sodium hydroxide (2×10 mL) and water (10 mL), then dried (MgSO₄). The drying agent removed by filtration and the solvent removed in vacuo to yield the crude title compound as a cream solid, which was purified by flash chromatography on silica, eluting with chloroform, then ethyl acetate, then methanol to give the pure title compound as an oil. M+1=453.3.

### EXAMPLE 56

### 1-[2-(4-(6-Fluoro-1H-indol-3-yl)-3,6-dihydro-1(2H)-pyridinyl)ethyl]-2-oxo-1,2,3,4-tetrahydro-6-quinolinecarboxamide

### a) 6-Bromo-3,4-dihydro-2(1H)-quinolinone

To a solution of 3,4-dihydro-2(1H)-quinolinone (30 g, 204 mmol) in dried DMF (250 mL) at 0° C. was slowly added N-bromosuccinimide (38 g, 1.05 eq) via a dropping funnel. The reaction mixture was stirred at room temperature overnight, then poured into cold water (3.5L) and the precipitate formed filtered off and dried in vacuo at 45° C. to give the title compound as a white solid.

### b) 2-Oxo-1,2,3,4-tetrahydro-6-quinolinecarboxylic acid

To dried THF (200 mL) under nitrogen was added 6-bromo-3,4-dihydro-2(1H)-quinolinone (5 g, 22.1 mmol). The solution was cooled to −78° C. and n-butyllithium (2.5M in THF) (29 mL, 3.3 eq) added, then stirred at −78° C. for 30 minutes. Nitrogen gas was passed through dry ice and into the reaction vessel for 10 minutes and then the reaction was allowed to warm to room temperature over 1 h. The mixture was quenched with saturated ammonium chloride solution and all non-acidic material was extracted into ethyl acetate (2×). The aqueous layer was acidified with 2M HCl, and the resultant precipitate filtered, washed and dried in vacuo at 45° C. to give the title compound as a white solid.

### c) Allyl 1-allyl-2-oxo-1,2,3,4-tetrahydro-6-quinolinecarboxylate

To dried DMF (40 mL) was added 2-oxo-1,2,3,4-tetrahydro-6-quinolinecarboxylic acid (3.5 g, 18.3 mmol). The reaction mixture was stirred at room temperature as sodium hydride (60% dispersion in oil) (2.2 g, 3 eq) was added, then left for 20 min. Allyl bromide was added (6.65 g, 3.5 eq) and the reaction stirred for a further 2 h. The mixture was poured into cold water and extracted with chloroform (3×), dried (MgSO₄) and concentrated in vacuo to afford a brown oil (3.5 g). The crude product was purified by flash chromatography on silica, eluting with chloroform/ethyl acetate (100:0 to 0:100), then ethyl acetate/acetone (50:50 to 0:100), to give the title compound as a pale yellow oil.

### d) 1-Allyl-2-oxo-1,2,3,4-tetrahydro-6-quinolinecarboxylic acid

To THF (30 mL) and water (30 mL) was added allyl 1-allyl-2-oxo-1,2,3,4-tetrahydro-6-quinolinecarboxylate (2.36 g, 8.7 mmol) and lithium hydroxide (0.21 g, 1 eq). The reaction mixture was stirred under nitrogen and heated under reflux for 24 h. The mixture was poured into saturated sodium bicarbonate solution and extracted with ethyl ether (2×). The aqueous layer was acidified with 2M HCl and the resultant precipitate filtered off, washed and dried in vacuo to yield the title compound as a white solid.

### e) 1-Allyl-3,4-dihydro-2(1H)-quinolinone-6-carboxamide

To dried DMF (40 mL) was added 1-allyl-2-oxo-1,2,3,4-tetrahydro-6-quinolinecarboxylic acid (0.38 g, 1.67 mmol) and carbonyldiimidazole (0.6 g, 2 eq). The reaction was stirred at room temperature overnight, then aqueous ammonia (0.880) (25 mL) was added and the reaction stirred for a further 24 h. The mixture was poured into cold water and extracted with chloroform (2×), dried (MgSO₄) and concentrated in vacuo to afford the title compound as a white solid (contaminated with imidazole).

### f) 2-Oxo-1-(2-oxoethyl)-1,2,3,4-tetrahydro-6-quinolinecarboxamide

To methanol (50 mL) was added 1-allyl-3,4-dihydro-2 (1H)-quinolinone-6-amide (0.37 g, 1.63 mmol) and the solution cooled to −78° C. Ozone was gently bubbled through the stirred reaction mixture until a blue colour persisted. The solution was purged with nitrogen and then stirred with dimethylsulfide (4 mL) for 2 h, allowing to warm to room temperature. The solvent was removed in vacuo and the crude oil purified by flash chromatography on silica, eluting with methanol/chloroform (8:92). The title compound was isolated as an off-white solid.

### g) 1-[2-(4-(6-Fluoro-1H-indol-3-yl)-3,6-dihydro-1(2H)-pyridinyl)ethyl]-2-oxo-1,2,3,4-tetrahydro-6-quinolinecarboxamide

2-oxo-1-(2-oxoethyl)-1,2,3,4-tetrahydro-6-quinolinecarboxamide (77 mg, 0.3 mmol) and 6-fluoro-1H-indol-3-ylpiperidine (13 mg, 2 eq) was stirred in methanol for 1 h. Sodium borohydride (3 mg, 1.3 eq) was added and then acetic acid (0.3 mL). The reaction mixture was stirred at room temperature for 18 h, then poured into saturated sodium bicarbonate solution and extracted with chloroform (2×). The combined organic extracts were dried (MgSO₄), filtered and evaporated in vacuo. The crude yellow oil was purified by preparative HPLC, eluting with acetontrile/water/aqueous ammonia (50:50:0.2) on a KR100-5 C18 reverse phase column, to yield the title compound. M+1=433.

### EXAMPLE 57

### 1-[2-(4-(6-Fluoro-1H-indol-3-yl)-3,6-dihydro-1(2H)-pyridinyl)ethyl]-N-methyl-2-oxo-1,2,3,4-tetrahydro-6-quinolinecarboxamide

### a) 1-Allyl-N-methyl-2-oxo-1,2,3,4-tetrahydro-6-quinolinecarboxamide

To a stirred solution of I-allyl-2-oxo-1,2,3,4-tetrahydro-6-quinolinecarboxylic acid (3.0 g, 12.9 mmol) in dry THF (150 mL) was added carbonyldiimidazole (4.2 g, 2 eq). After 1 hour methylamine (2 eq, 0.8 g) (solution in THF) was added. The reaction mixture was stirred under a dry atmosphere for 3 days then added to saturated bicarbonate solution and extracted with chloroform (2×). The combined organic extracts were dried (MgSO₄), filtered and concentrated in vacuo to give the title compound as a yellow oil.

### b) N-Methyl-2-oxo-1-(2-oxoethyl)-1,2,3,4-tetrahydro-6-quinolinecarboxamide

To a solution of 1-allyl-N-methyl-2-oxo-1,2,3,4-tetrahydro-6-quinolinecarboxamide (4.05 g, 16.6 mmol) in THF (50 mL) and water (50 mL) was added sodium periodate (7.1 g, 2 eq) and stirred until dissolved. Osmium tetraoxide was added (one crystal) and the mixture stirred at room temperature overnight. The solvent was removed in vacuo, water added and extracted with chloroform (3×). The combined organic extracts were dried (MgSO₄), filtered and concentrated in vacuo to give a yellow oil. Trituration with diethyl ether gave the title compound as a white solid.

### c) 1-[2-(4-(6-Fluoro-1H-indol-3-yl)-3,6-dihydro-1(2H)-pyridinyl)ethyl]-N-methyl-2-oxo-1,2,3,4-tetrahydro-6-quinolinecarboxamide

N-methyl-2-oxo-1-(2-oxoethyl)-1,2,3,4-tetrahydro-6-quinolinecarboxamide (0.321 g, 1.3 mmol) and 6-fluoro-1H-indol-3-ylpiperidine (337 mg, 1.2 eq) was stirred at room temperature in methanol (5 mL) for 1 h. Sodium cyanoborohydride (90 mg, 1.1 eq), then acetic acid (1 mL) was added. The reaction mixture was stirred at room temperature overnight. The solvent was removed in vacuo and the resultant oil purified by flash chromatography on silica, eluting with methanol/chloroform (10:90) to give the title compound as a yellow solid. M+1=447.

Preparation

### (1S)-2-[6-Aminocarbonyl)-3,4-dihydro-1H-2-benzopyran-1-yl]ethyl methanesulfonate

Prepared as described above for the racemic 2-[6-aminocarbonyl)-3,4-dihydro-1H-2-benzopyran-1-yl]ethyl methanesulfonate, but commencing with (1S)-2-(6-bromo-3,4-dihydro-1H-2-benzopyran-1-yl)ethanol (prepared according to the procedure of TenBrink et al., J. Med. Chem., 1996, 39, 2435-2437).

### EXAMPLE 58

### (1S)-1-{2-[(2R)-4-(6-Fluoro-1-naphthyl)-2-methylpiperazinyl]ethyl}-3,4-dihydro-1H-2-benzopyran-6-carboxamide

### a) 6-Fluoro-3,4-dihydro-1-naphthalenyl trifluoromethanesulfonate

To a stirred solution of 6-fluoro-3,4-dihydro-1(2H)-naphthalenone (0.50 g, 3 mmol) in dry THF (25 mL) at −78° C. under nitrogen was added lithium bis(trimethylsilyl)amide (1M in THF) (3.6 mL, 3.6 mmol) over 5 min. The solution was stirred for 1 h, then N-phenyltrifluoromethanesulfonimide (1.3 g, 3.6 mmol) was added in one portion and the reaction mixture allowed to warm to room temperature. Stirring was continued for 2 h, then the solvent was removed in vacuo. The residue was dissolved in ethyl acetate and washed with 2M sodium hydroxide, water, and then brine. The organic extracts were dried (MgSO₄), and concentrated in vacuo. The resultant red oil was purified by column chromatography on silica, eluting with ethyl acetate/hexane (1:9), to yield 6-fluoro-3,4-dihydro-1-naphthalenyl trifluoromethane sulfonate as a colourless oil.

### b) 6-Fluoro-1-naphthyl trifluoromethanesulfonate

To a solution of 6-fluoro-3,4-dihydro-1-naphthalenyl trifluoromethane sulfonate (0.77 g, 2.8 mmol) in dioxan (15 mL) was added 2,3-dichloro-5,6-dicyano-1,4-benzoquinone (0.95 g, 4.2 mmol) and the reaction mixture heated under reflux for 18 h. The solvent was removed in vacuo and the crude product purified by column chromatography on silica, eluting with hexane, to yield 6-fluoro-1-naphthyl trifluoromethanesulfonate as a white solid.

### c) (3R)-1-(6-Fluoro-1-naphthyl)-3-methylpiperazine

To a solution of 6-fluoro-1-naphthyl trifluoromethanesulfonate (0.29 g, 1 mmol) in toluene (2 mL) under nitrogen was added (2R)-methylpiperazine (0.10 g, 1.2 mmol), (R)-2,2′-bis(diphenylphosphino)-1,1′-binaphthyl (47 mg, 0.075 mmol), palladium (II) acetate (11 mg, 0.05 mmol) and cesium carbonate (0.46 g, 1.4 mmol). The resulting suspension was heated at 110° C. for 16 h. Upon cooling, the mixture was filtered through a short celite pad (washing with ethyl acetate), the filtrate concentrated in vacuo and the crude product purified by flash column chromatography on silica gel, eluting with dichloromethane/methanol (7:3), to yield (3R)-1-(6-fluoro-1-naphthyl)-3-methylpiperazine as a brown oil.

### d) (1S)-1-{2-[(2R)-4-(6-Fluoro-1-naphthyl)-2-methylpiperazinyl]ethyl)-3,4-dihydro-1H-2-benzopyran-6-carboxamide

(3R)-1-(6-Fluoro-1-naphthyl)-3-methylpiperazine was coupled with (1S)-2-[6-aminocarbonyl)-3,4-dihydro-1H-2-benzopyran-1-yl]ethyl methanesulfonate, as described for Example 1 c), to yield (1S)-1-{2-[(2R)-4-(6-fluoro-1-naphthyl)-2-methylpiperazinyl]ethyl}-3,4-dihydro-1H-2-benzopyran-6-carboxamide. M+H=448.

The following examples were prepared by substituting (2R)-methylpiperazine in the above example with alternatively 2-substituted piperazines (prepared according-to the procedure of Mickelson et al., J. Org. Chem., 1995, 60, 4177-4183):

### EXAMPLE 59

### (1S)-1-{2-[(2R)-4-(6-Fluoro-1-naphthyl)-2-ethylpiperazinyl]ethyl)-3,4-dihydro-1H-2-benzopyran-6-carboxamide

Prepared with (2R)-ethylpiperazine. M+H=462.

### EXAMPLE 60

### (1S)-1-{2-[(2S)-4-(6-Fluoro-1-naphthyl)-2-ethylpiperazinyl]ethyl}-3,4-dihydro-1H-2-benzopyran-6-carboxamide

Prepared with (2S)-ethylpiperazine. M+H=462.

### EXAMPLE 61

### (1S)-1-{2-[(2R)-4-(6-Fluoro-1-naphthyl)-2-propylpiperazinyl]ethyl}-3,4-dihydro-1H-2-benzopyran-6-carboxamide

Prepared with (2R)-propylpiperazine. M+H=476.

### EXAMPLE 62

### (1S)-1-{2-[(4-(6-Fluoro-1-naphthyl)-2-(methoxymethyl)piperazinyl]ethyl}-3,4-dihydro-1H-2-benzopyran-6-carboxamide

### a) 1,4-Bis(tert-butoxycarbonyl)-2-piperazinecarboxylic acid

To a solution of piperazine-2-carboxylic acid dihydrochloride (5 g, 24.6 mmol) in 2M sodium hydroxide (40 mL) and ethanol (40 mL) was added di-tert-butyl dicarbonate (11.82 g, 54.1 mmol) and the reaction mixture stirred for 3 days. The organic solvent was removed in vacuo, the aqueous phase basified with 2M sodium hydroxide and extracted with diethyl ether to remove excess di-tert-butyl dicarbonate. The aqueous layer was adjusted to pH 3-4 and extracted with ethyl acetate. The combined organic extracts were dried (MgSO₄), filtered and evaporated to yield 1,4-bis(tert-butoxycarbonyl)-2-piperazinecarboxylic acid as a white solid.

### b) Di(tert-butyl) 2-(hydroxymethyl)-1,4-piperazinedicarboxylate

To a stirred solution of 1,4-bis(tert-butoxycarbonyl)-2-piperazinecarboxylic acid (2 g, 6.1 mmol) in dry THF (50 mL) at 0° C. was added borane dimethyl sulfide (1.52 mL, 18.9 mmol), and the reaction mixture allowed to warm to room temperature. The reaction was monitored by tlc and when all starting material was consumed, the mixture was cooled in ice-water and quenched by careful addition of water. The product was extracted into ethyl acetate, the combined organic extracts washed with brine, dried (MgSO₄), filtered and evaporated in vacuo to yield di(tert-butyl) 2-(hydroxymethyl)-1,4-piperazinedicarboxylate as a white solid.

### c) Di(tert-butyl) 2-(methoxymethyl)-1,4-piperazinedicarboxylate

To a stirred solution of di(tert-butyl) 2-(hydroxymethyl)-1,4-piperazinedicarboxylate (0.47 g, 1.5 mmol) in DMF (10 mL) at 0° C. under nitrogen was added sodium hydride (60% dispersion in oil) (89 mg, 2.25 mmol) in portions. The resultant suspension was allowed to warm to room temperature. After 30 min iodomethane (0.186 mL, 3 mmol) was added and the reaction mixture left to stir for 18 h. The crude mixture was extracted from water into ethyl acetate, the combined organic extracts dried (MgSO₄), filtered and evaporated in vacuo, to yield di(tert-butyl) 2-(methoxymethyl)-1,4-piperazinedicarboxylate.

### d) 2-(Methoxymethyl)piperazine

To a stirred solution of di(tert-butyl) 2-(methoxymethyl)-1,4-piperazine dicarboxylate (0.50 g, 1.5 mmol) in dichloromethane (10 mL) at 0° C. was added trifluoroacetic acid (3 mL). The reaction mixture was allowed to warm to room temperature and stirred for 3 h. The mixture was concentrated in vacuo and triturated with diethyl ether to yield the trifluoracetic acid salt of the required product as a white solid. The free base, 2-(methoxymethyl)piperazine, was liberated by elution in methanol through an ion exchange column.

### e) 1-(6-Fluoro-1-naphthyl)-3-(methoxymethyl)piperazine

2-(Methoxymethyl)piperazine was coupled with 6-fluoro-1-naphthyl trifluoromethanesulfonate, as described for Example 58 c), to yield 1-(6-fluoro-1-naphthyl)-3-(methoxymethyl)piperazine.

### f) (1S)-1-{2-[4-(6-Fluoro-1-naphthyl)-2-(methoxymethyl)piperazinyl)ethyl}-3,4-dihydro-1H-2-benzopyran-6-carboxamide

1-(6-Fluoro-1-naphthyl)-3-(methoxymethyl)piperazine was coupled with (2S)-2-[6-(aminocarbonyl)-3,4-dihydro-1H-2-benzopyran-1-yl]ethyl methanesulfonate, as described for Example 58 d), to yield (1S)-1-{2-[4-(6-fluoro-1-naphthyl)-2-(methoxymethyl)piperazinyl]ethyl}-3,4-dihydro-1H-2-benzopyran-6-carboxamide. M+H=478.

### EXAMPLE 63

### (1S)-1-{2-[(2R)-4-(6-Cyano-1-naphthyl)-2-methylpiperazinyl]ethyl)-3,4-dihydro-1H-2-benzopyran-6-carboxamide

### a) 5-Bromo-2-naphthonitrile

To a solution of 5-bromo-2-naphthoic acid (4.3 g, 17 mmol)) in dry pyridine (75 mL) at 0° C. was added methanesulfonyl chloride (1.4 mL, 18 mmol). After stirring at 0° C. for 1 h, ammonia gas was bubbled through the solution for 10 min, whilst maintaining the temperature below 5° C. During the gas addition the solution became viscous, so additional dry pyridine (˜30 mL) was added. Excess ammonia was removed in vacuo, the solution again cooled to 0° C., then treated with additional methanesulfonyl chloride (12.5 mL) and allowed to warm to room temperature overnight. The solution was poured onto ice cold water, the mixture stirred for 30 min and the brown precipitate collected by filtration, washed on the sinter with ice cold water, then dried in vacuo. The crude product was dissolved in hot chloroform (˜35 mL) and insoluble material filtered off. The chloroform was removed and the residue dissolved in a minimum volume of ether at reflux. Hexane was added until the solution remained turbid at reflux, the solution filtered rapidly into a pre-heated flask, and allowed to cool slowly to room temperature. The precipitate was collected by filtration, washed with hexane, and dried in vacuo, to yield 5-bromo-2-naphthonitrile. Further crops were obtained by cooling the filtrate at −18° C. overnight.

### b) (3R)-1-(6-Cyano-1-naphthyl)-3-methylpiperazine

To a solution of 5-bromo-2-naphthonitrile (0.47 g, 2 mmol) in dry toluene (30 mL) was added tris(dibenzylideneacetone)dipalladium (0) (40 mg), (R)-2,2′-bis(diphenylphosphino)-1,1′-binaphthyl (82 mg), (2R)-methylpiperazine (0.24 g, 2.4 mmol) and sodium tert-butoxide (0.27 g, 2.8 mmol). The solution was evacuated until bubbling started, then the atmosphere replaced with nitrogen. This purging and evacuation procedure was repeated for 15 min, then the mixture heated under reflux for 8 h. The reaction mixture was cooled to room temperature, diluted with ethyl acetate and filtered through celite. The filtrate was washed with aqueous ammonia, dried (MgSO₄), filtered and evaporated in vacuo. The residue was dissolved in methanol (10 mL) and applied to an activated SCX cartridge (10 g). The cartridge was washed with methanol (100 mL), then the product isolated by elution with 2M ammonia in methanol (50 mL). The solvent was removed in vacuo and further purified by flash chromatography on silica, eluting with acetone, to yield (3R)-1-(6-cyano-1-naphthyl)-3-methylpiperazine.

### c) (1S)-1-{2-[(2R)-4-(6-Cyano-1-naphthyl)-2-methylpiperazinyl]ethyl)-3,4-dihydro-1H-2-benzopyran-6-carboxamide

(3R)-1-(6-Cyano-1-naphthyl)-3-methylpiperazine was coupled with (1S)-2-[6-aminocarbonyl)-3,4-dihydro-1H-2-benzopyran-1-yl]ethyl methanesulfonate, as described for Example 58 d), to yield (1S)-1-{2-[(2R)-4-(6-cyano-1-naphthyl)-2-methylpiperazinyl]ethyl)-3,4-dihydro-1H-2-benzopyran-6-carboxamide. M+H=455.

The following examples were prepared by substituting (2R)-methylpiperazine in the above example with alternatively 2-substituted piperazines:

### EXAMPLE 64

### (1S)-1-{2-[(2R)-4-(6-Cyano-1-naphthyl)-2-ethylpiperazinyl]ethyl}-3,4-dihydro-1H-2-benzopyran-6-carboxamide

Prepared using (2R)-ethylpiperazine. M+H=469.

### EXAMPLE 65

### (1S)-1-{2-[(2S)-4-(6-Cyano-1-naphthyl)-2-ethylpiperazinyl]ethyl}-3,4-dihydro-1H-2-benzopyran-6-carboxamide

Prepared using (2S)-ethylpiperazine. M+H=469.

### EXAMPLE 66

### (1S)-1-{2-[4-(6-cyano-1-naphthyl)piperazinyl]ethyl}-3,4-dihydro-1H-2-benzopyran-6-carboxamide

Prepared using piperazine. M+H=441.

### EXAMPLE 67

### (1S)-1-{2-[4-(6-Cyano-1-naphthyl)hexahydro-1H-1,4-diazepin-1-yl]ethyl}-3,4-dihydro-1H-2-benzopyran-6-carboxamide

Prepared using homopiperazine. M+H=455.

The following examples were prepared as described for Example 63, substituting 5-bromo-2-naphthonitrile with alternative substituted bromonaphthalenes:

### EXAMPLE 68

### (1S)-1-{2-[(2R)-4-(4-Fluoro-1-naphthyl)-2-methylpiperazinyl]ethyl}-3,4-dihydro-1H-2-benzopyran-6-carboxamide

Prepared from 1-fluoro-4-bromonaphthalene and (2R)-methylpiperazine. M+H=448.

### EXAMPLE 69

### (1S)-1-{2-[(2R)-4-(4-Methyl-1-naphthyl)-2-methylpiperazinyl]ethyl}-3,4-dihydro-1H-2-benzopyran-6-carboxamide

Prepared from 1-bromo-4-methylnaphthalene and (2R)-methylpiperazine. M+H=444.

### EXAMPLE 70

### (1S)-1-{2-[(2R)-4-(2-Naphthyl)-2-methylpiperazinyl]ethyl}-3,4-dihydro-1H-2-benzopyran-6-carboxamide

Prepared from 2-bromonaphthalene and (2R)-methylpiperazine. M+H=430.

### EXAMPLE 71

### (1S)-1-{2-[4-(2-Naphthyl)piperazinyl]ethyl}-3,4-dihydro-1H-2-benzopyran-6-carboxamide

Prepared from 2-bromonaphthalene and piperazine. M+H=416.

### EXAMPLE 72

### (1S)-1-{2-[(2R)-4-(4-Chloro-1-naphthyl)-2-methylpiperazinyl]ethyl}-3,4-dihydro-1H-2-benzopyran-6-carboxamide

### a) 4-Chloro-1-naphthyl trifluoromethanesulfonate

To 4-chloro-1-naphthol (0.60 g, 3.36 mmol) in dry THF (40 mL) at 0° C. was added sodium tert-butoxide (0.355 g, 3.7 mmol) and the solution stirred for 10 min. N-phenyltrifluoromethanesulfonimide (1.32 g, 3.7 mmol) was added and the reaction mixture allowed to warm to room temperature over 2.5 h. Water was added and the organic solvent removed in vacuo. The residue was extracted into ethyl acetate, the combined organic extracts washed with saturated aqueous sodium hydrogen carbonate, dried (MgSO₄) and evaporated in vacuo. The crude product was purified by column chromatography on silica, eluting with ethyl acetate/hexane (1:4), to yield 4-chloro-1-naphthyl trifluoromethanesulfonate as a colourless oil.

### b) (3R)-1-(4-Chloro-1-naphthyl)-3-methylpiperazine

4-Chloro-1-naphthyl trifluoromethanesulfonate was coupled with (2R)-methylpiperazine as described for Example 58 c), to yield (3R)-1-(4-chloro-1-naphthyl)-3-methylpiperazine.

### c) (1S)-1-{2-[(2R)-4-(4-Chloro-1-naphthyl)-2-methylpiperazinyl]ethyl}-3,4-dihydro-1H-2-benzopyran-6-carboxamide

(3R)-1-(4-Chloro-1-naphthyl)-3-methylpiperazine was coupled with (1S)-2-[6-aminocarbonyl)-3,4-dihydro-1H-2-benzopyran-1-yl]ethyl methanesulfonate as described for Example 58 d), to yield (1S)-1-{2-[(2R)-4-(4-chloro-1-naphthyl)-2-methylpiperazinyl]ethyl}-3,4-dihydro-1H-2-benzopyran-6-carboxamide. M+H=464.

### EXAMPLE 73

### (1S)-1-{2-[(2R)-4-(4-Cyano-1-naphthyl)-2-methylpiperazinyl]ethyl}-3,4-dihydro-1H-2-benzopyran-6-carboxamide

### a) 4-Bromo-1-naphthonitrile

4-Amino-1-naphthonitrile (0.81 g, 4.8 mmol) was suspended in conc. hydrochloric acid (24 mL), sonicated for 5 min and cooled to 0° C. A solution of sodium nitrite (0.5 g, 7.2 mmol) in water (4 mL) was added dropwise with stirring, maintaining the temperature below 5° C. After stirring for 45 min at 0° C., the reaction mixture was added dropwise to copper(I)bromide (3.47 g, 24.2 mmol) in water (10 mL) at 0° C., then stirred at room temperature for 1 day. Water was added and extracted with diethyl ether. The combined organic extracts were washed with saturated aqueous sodium hydrogen carbonate, then dried (MgSO₄), filtered and evaporated in vacuo. The crude product was purified by flash chromatography on silica, eluting with ethyl acetate/hexane (1:4), to yield 4-bromo-1-naphthonitrile as an oil.

### b) (3R)-1-(4-Cyano-1-naphthyl)-3-methylpiperazine

4-Bromo-1-naphthonitrile was coupled with (2R)-methyl piperazine as described for Example 63 b), to yield (3R)-1-(4-cyano-1-naphthyl)-3-methylpiperazine.

### c) (1S)-1-{2-[(2R)-4-(4-Cyano-1-naphthyl)-2-methylpiperazinyl]ethyl}-3,4-dihydro-1H-2-benzopyran-6-carboxamide

(3R)-1-(4-Cyano-1-naphthyl)-3-methylpiperazine was coupled with (1S)-2-[6-aminocarbonyl)-3,4-dihydro-1H-2-benzopyran-1-yl]ethyl methanesulfonate as described for Example 58 d), to yield (1S)-1-{2-[(2R)-4-(4-cyano-1-naphthyl)-2-methylpiperazinyl]ethyl}-3,4-dihydro-1H-2-benzopyran-6-carboxamide. M+H=455.

### EXAMPLE 74

### (1S)-1-{2-[(2R)-4-(4,6-Difluoro-1-naphthyl)-2-methylpiperazinyl]ethyl}-3,4-dihydro-1H-2-benzopyran-6-carboxamide

### a) (R)-1-(6-Fluoro-4-iodo-1-naphthyl)-3-methylpiperazine

To a stirred solution of (R)-1-(6-fluoro-1-naphthyl)-3-methylpiperazine (0.626 g, 2.56 mmol) in dichloromethane (25 mL) at −25° C. was added bis(pyridine)iodonium(I)tetrafluoroborate (1.05 g, 2.82 mmol) and tetrafluoroboric acid (54% solution in ether) (0.78 mL, 5.64 mmol). After 30 min the reaction was quenched by addition of 10% aqueous sodium carbonate. The phases were separated and the aqueous phase extracted with dichloromethane. The combined organic extracts were dried (Na₂SO₄) and evaporated to dryness. The residue was purified by column chromatography on silica gel, eluting with dichloromethane/methanol (9:1), to yield (R)-1-(6-fluoro-4-iodo-1-naphthyl)-3-methylpiperazine as a brown solid.

### b) Benzyl (R)-4-(6-Fluoro-4-iodo-1-naphthyl)-2-methylpiperazine-1-carboxylate

To a solution of (R)-1-(6-fluoro-4-iodo-1-naphthyl)-3-methylpiperazine (0.25 g, 0.675 mmol) and triethylamine (0.14 mL, 1.01 mmol) in dichloromethane (3 mL) at 0° C. under nitrogen was added benzyl chloroformate (0.12 mL, 0.81 mmol). The reaction mixture was stirred for 3 h, then the solvent removed in vacuo. The crude product was purified by column chromatography on silica gel, eluting with hexane/ethyl acetate (8:2), to yield benzyl (R)-4-(6-fluoro-4-iodo-1-naphthyl)-2-methylpiperazine-1-carboxylate as a yellow solid.

### c) Benzyl (R)-4-(4,6-difluoro-1-naphthyl)-2-methylpiperazine-1-carboxylate

To a stirred solution of benzyl (R)-4-(6-fluoro-4-iodo-1-naphthyl)-2-methylpiperazine-1-carboxylate (0.30 g, 0.595 mmol) in dry tetrahydrofuran (6 mL) at −78° C. under nitrogen was added tert-butillithium (1.5M solution in pentane) (0.87 mL, 1.31 mmol). The reaction mixture was stirred at this temperature for 15 min, then N-fluorobenzenesulfonimide (0.225 g, 0.714 mmol) added in one portion. The reaction was warmed to 0° C. over 2 h, then quenched with water. The mixture was extracted with dichloromethane, and the combined organic extracts dried (Na₂SO₄) and evaporated to dryness. The residue was purified by column chromatography on silica gel, eluting with hexane/ethyl acetate (8:2) to yield the required product contaminated with the monofluorinated material (approx. 1:1 ratio). This crude product was further purified by HPLC on a Kromasil Si60 column (20×250 mm), eluting with hexane/acetone (95:5), to yield benzyl (R)-4-(4,6-difluoro-1-naphthyl)-2-methylpiperazine-1-carboxylate.

### d) (R)-1-(4,6-Difluoro-1-naphthyl)-3-methylpiperazine

A mixture of (R)-4-(4,6-difluoro-1-naphthyl)-2-methylpiperazine-1-carboxylate (45 mg, 0.113 mmol) and 10% palladium over charcoal (50 mg) in methanol (2 mL) was stirred under a hydrogen atmosphere for 1 h. The solvent was removed in vacuo and the residue purified by column chromatography on silica gel, eluting with dichloromethane/methanol (9:1), to yield (R)-1-(4,6-difluoro-1-naphthyl)-3-methylpiperazine.

### e) (1S)-1-{2-[(2R)-4-(4,6-Difluoro-1-naphthyl)-2-methylpiperazinyl]ethyl}-3,4-dihydro-1H-2-benzopyran-6-carboxamide

(R)-1-(4,6-Difluoro-1-naphthyl)-3-methylpiperazine was coupled with (1S)-2-[6-aminocarbonyl)-3,4-dihydro-1H-2-benzopyran-1-yl]ethyl methanesulfonate as described for Example 58 d), to yield (1S)-1-{2-[(2R)-4-(4,6-difluoro-1-naphthyl)-2-methylpiperazinyl]ethyl}-3,4-dihydro-1H-2-benzopyran-6-carboxamide. M+H=466.

### EXAMPLE 75

### (1S)-1-{2-[(2R)-4-(4-Cyano-6-fluoro-1-naphthyl)-2-methylpiperazinyl]ethyl}-3,4-dihydro-1H-2-benzopyran-6-carboxamide

### a) (3R)-1-(4-Cyano-6-fluoro-1-naphthyl)-3-methylpiperazine

A mixture of (3R)-1-(6-fluoro-4-iodo-1-naphthyl)-3-methylpiperazine (0.26 g, 0.70 mmol), potassium cyanide (50 mg, 0.74 mmol), copper (I) iodide (13 mg, 0.067 mmol) and tetrakis(triphenylphosphine)palladium (0) (39 mg, 0.034 mmol) in tetrahydrofuran (5 mL) was heated at 100° C. under nitrogen in a sealed tube for 20 h. The solvent was removed in vacuo and the residue purified by column chromatography on silica gel, eluting with dichloromethane/methanol (9:1), to yield (3R)-1-(4-cyano-6-fluoro-1-naphthyl)-3-methylpiperazine as a brown solid.

### b) (1S)-1-{2-[(2R)-4-(4-Cyano-6-fluoro-1-naphthyl)-2-methylpiperazinyl]ethyl}-3,4-dihydro-1H-2-benzopyran-6-carboxamide

(3R)-1-(4-Cyano-6-fluoro-1-naphthyl)-3-methylpiperazine was coupled with (1S)-2-[6-aminocarbonyl)-3,4-dihydro-1H-2-benzopyran-1-yl]ethyl methanesulfonate as described for Example 58 d). The crude product was purified by column chromatography on silica gel, eluting with dichloromethane/methanol (92:8), to yield (1S)-1-{2-[(2R)-4-(4-cyano-6-fluoro-1-naphthyl)-2-methylpiperazinyl]ethyl}-3,4-dihydro-1H-2-benzopyran-6-carboxamide as a yellow solid. M+H=473.

### EXAMPLE 76

### (1S)-1-{2-[(2R)-4-(4,5-Dimethyl-1-naphthyl)-2-methylpiperazinyl]ethyl}-3,4-dihydro-1H-2-benzopyran-6-carboxamide

### a) 6-Bromo-1H,3H-naphtho[1,8-cd]pyran

To 6-bromo-1H,3H-naphtho[1,8-cd]pyran-1,3-dione (1.5 g, 5.4 mmol) in ethanol (10 mL) was added sodium borohydride (0.41 g, 10.8 mmol) and the mixture stirred at room temperature for 1 h. The reaction was quenched with 3M hydrochloric acid and extracted into dichloromethane. The combined organic extracts were dried (Na₂SO₄) and concentrated in vacuo. The residue was dissolved in dichloromethane and trifluoroacetic acid (1.05 mL, 13.53 mmol) and triethylsilane (4.3 mL, 27 mmol) added. After stirring for 5 min at room temperature, the solvent was removed in vacuo and the residue purified by column chromatography on silica gel, eluting with dichloromethane, to yield 6-bromo-1H,3H-naphtho[1,8-cd]pyran as a white solid.

### b) 1-Bromo-4,5-bis(bromomethyl)naphthalene

To a solution of 6-bromo-1H,3H-naphtho[1,8-cd]pyran (0.62 g, 2.49 mmol) in dichloromethane (25 mL) was added boron tribromide (1M solution in dichloromethane) (2.74 mL, 2.74 mmol) and the reaction mixture heated at reflux for 30 min. After quenching with water, the organic extract was dried (Na₂SO₄) and concentrated to dryness, to yield 1-bromo-4,5-bis(bromomethyl)naphthalene as a white solid.

### c) 1-Bromo-4,5-dimethylnaphthalene

To a solution of 1-bromo-4,5-bis(bromomethyl)naphthalene (0.10 g, 0.254 mmol) and sodium borohydride (20 mg, 0.51 mmol) in dimethylformamide (2 mL) was added silver nitrate (87 mg, 0.51 mmol). After 5 min the reaction mixture was poured into water and extracted into dichloromethane. The combined organic extracts were dried (Na₂SO₄) and evaporated to dryness, to yield 1-bromo-4,5-dimethylnaphthalene as a pale yellow solid.

### d) (3R)-1-(4,5-Dimethyl-1-naphthyl)-3-methylpiperazine

1-Bromo-4,5-dimethylnaphthalene was coupled with (2R)-methylpiperazine, as described for Example 63 b), to yield (3R)-1-(4,5-dimethyl-1-naphthyl)-3-methylpiperazine as a brown oil.

### e) (1S)-1-{2-[(2R)-4-(4,5-Dimethyl-1-naphthyl)-2-methylpiperazinyl]ethyl}-3,4-dihydro-1H-2-benzopyran-6-carboxamide

(3R)-1-(4,5-Dimethyl-1-naphthyl)-3-methylpiperazine was coupled with (1S)-2-[6-aminocarbonyl)-3,4-dihydro-1H-2-benzopyran-1-yl]ethyl methanesulfonate as described for Example 58 d), to yield (1S)-1-{2-[(2R)-4-(4,5-dimethyl-1-naphthyl)-2-methylpiperazinyl]ethyl}-3,4-dihydro-1H-2-benzopyran-6-carboxamide. M+H=458.

### EXAMPLE 77

### (1S)-1-{2-[(2R)-4-(6-Fluoro-2-naphthyl)-2-methylpiperazinyl]ethyl)-3,4-dihydro-1H-2-benzopyran-6-carboxamide

### a) [(6-Bromo-2-naphthyl)oxy](tert-butyl)dimethylsilane

To a solution of 6-bromo-2-naphthol (0.50 g, 2.2 mmol) in dry DMF (6 mL) under nitrogen was added tert-butyldimethylsilyl chloride (0.506 g, 3.4 mmol) and imidazole (0.229 g, 3.4 mmol) and the mixture stirred at room temperature overnight. Water and diethyl ether were added and the layers were separated. The organic layer was washed with brine, dried (MgSO₄), filtered and concentrated in vacuo. The crude product was purified by flash chromatography on silica, eluting with hexane/ethyl acetate (3:1), to yield the title compound.

### b) [(6-Fluoro-2-naphthyl)oxy](tert-butyl)dimethylsilane

[(6-Bromo-2-naphthyl)oxy](tert-butyl)dimethylsilane (0.682 g, 2.0 mmol) was dissolved in dry THF (20 mL) under nitrogen and cooled to −78° C. Butyllithium (1.6M in hexane) (1.4 mL, 2.2 mmol) was added dropwise and the mixture stirred at this temperature for 20 min. N-Fluoro-bis-phenylsulfonimide (0.765 g, 2.4 mmol) was added and the reaction stirred at room temperature for 4 h. Water and dichloromethane were added and the layers separated. The organic layer was washed with brine, dried (MgSO₄), filtered and concentrated in vacuo. The crude product was purified by flash chromatography on silica, eluting with hexane/ethyl acetate (3:1), to yield the title compound.

### c) 6-Fluoro-2-naphthol

[(6-Fluoro-2-naphthyl)oxy](tert-butyl)dimethylsilane (0.10 g, 0.4 mmol) was dissolved in THF (2 mL). Tetrabutylammonium fluoride (1M in THF) (0.4 mL, 0.4 mmol) was added and the mixture stirred at room temperature for 30 min. The solvent was removed in vacuo and the residue purified by flash chromatography on silica, eluting with hexane/ethyl acetate (2:1), to yield the title compound as a white solid.

### d) 6-Fluoro-2-naphthyl trifluoromethanesulfonate

6-Fluoro-2-naphthol (56 mg, 0.3 mmol) was dissolved in dry THF (4 mL) under nitrogen and potassium tert-butoxide (37 mg, 0.33 mmol) added in one portion. After stirring for 10 min, N-phenyl-bis-trifluoromethylsulfonimide (118 mg, 0.33 mmol) was added and the mixture stirred at room temperature for 1 h. Water and hexane were added and the layers separated.

The organic layer was washed with 10% aqueous sodium carbonate, dried (MgSO₄), filtered and concentrated in vacuo. The crude product was purified by column chromatography on silica, eluting with hexane/ethyl acetate (2:1), to yield the title compound.

### e) (3R)-1-(6-Fluoro-2-naphthyl)-3-methylpiperazine

In an oven-dried flask provided with a reflux condenser, (2R)-methylpiperazine (45.7 mg, 0.5 mmol), palladium acetate (4.3 mg, 0.02 mmol), (±)-2,2′-bis(diphenylphosphino)-1,1′-binaphthyl (17.7 mg, 0.03 mmol) and cesium carbonate (173 g, 0.5 mmol) were suspended in dry toluene (0.8 mL). To this mixture was added 6-fluoro-2-naphthyl trifluoromethanesulfonate (112 mg, 0.4 mmol) in dry toluene (0.8 mL) by cannula and the reaction degassed by three cycles of vacuum-nitrogen. The reaction mixture was heated under reflux overnight, then cooled to room temperature and the solvent removed in vacuo. The residue was purified by flash chromatography on silica, eluting with dichloromethane/methanol (9:1), to yield the title compound.

### f) (1S)-1-{2-[(2R)-4-(6-Fluoro-2-naphthyl)-2-methylpiperazinyl]ethyl}-3,4-dihydro-1H-2-benzopyran-6-carboxamide

(3R)-1-(6-Fluoro-2-naphthyl)-3-methylpiperazine was reacted with (1S)-2-[6-aminocarbonyl)-3,4-dihydro-1H-2-benzopyran-1-yl]ethyl methanesulfonate as described for Example 58 d), to yield (1S)-1-{2-[(2R)-4-(6-fluoro-2-naphthyl)-2-methylpiperazinyl]ethyl}-3,4-dihydro-1H-2-benzopyran-6-carboxamide. M+H=448.

### EXAMPLE 78

### (1S)-1-{2-[4-(6-Fluoro-2-naphthyl)piperazinyl]ethyl}-3,4-dihydro-1H-2-benzopyran-6-carboxamide

Prepared as described above for Example 77, substituting piperazine for (2R)-methylpiperazine, to yield (1S)-1-{2-[4-(6-fluoro-2-naphthyl)piperazinyl)ethyl}-3,4-dihydro-1H-2-benzopyran-6-carboxamide. M+H=434.

### EXAMPLE 79

### (1S)-1-{2-[(2R)-4-(5-Fluoro-2-naphthyl)-2-methylpiperazinyl]ethyl}-3,4-dihydro-1H-2-benzopyran-6-carboxamide

### a) 5-Amino-2-naphthyl trifluoromethanesulfonate

To a solution of 5-amino-2-naphthol (1.433 g, 9.0 mmol) in dry THF (24 mL) under nitrogen at 0° C. was added sodium tert-butoxide (0.952 g, 9.90 mmol) and the resulting solution stirred for 1 h at 0° C. N-Phenyl-bis-trifluoromethylsulfonimide (3.54 g, 9.90 mmol) was added, the cooling bath removed, and the mixture stirred for 2 h. The resulting solution was diluted with ethyl acetate and washed with water, dilute aqueous sodium hydrogen carbonate, and brine. The organic phase was dried (MgSO₄), filtered, concentrated in vacuo, and the residue purified by flash chromatography on silica gel, eluting with hexane/ethyl acetate (3:1), to yield the title intermediate as a pale red solid.

### b) 5-Iodo-2-naphthyl trifluoromethanesulfonate

A suspension of 5-amino-2-naphthyl trifluoromethanesulfonate (9.50 g, 32.6 mmol) in conc. aqueous hydrochloric acid (160 mL) and water (23 mL) was sonicated for 5 min, then cooled to 0° C. and rapidly stirred. To this suspension was added dropwise a solution of sodium nitrite (3.38 g, 48.9 mmol) in water (40 mL), over a period of 20 min. The resulting brown mixture was stirred at 0° C. for 1 h. In a separate flask, potassium iodide (27.1 g, 163 mmol) was dissolved in water (55 mL), chilled to 0° C. in an ice bath, and rapidly stirred. To the KI solution was added dropwise the diazonium salt mixture, over a period of 45 min. The resulting brown mixture was stirred at 0° C. for 30 min, then allowed to warm to ambient temperature with stirring overnight. The mixture was diluted with water and the product extracted into diethyl ether. The ether extract was washed with water and brine, then dried (MgSO₄), filtered, concentrated in vacuo, and the residue purified by flash chromatography on silica gel, eluting with hexane/dichloromethane (9:1), to yield the title intermediate as a pale off-white solid.

### c) 5-Fluoro-2-naphthyl trifluoromethanesulfonate

To a stirred solution of 5-iodo-2-naphthyl trifluoromethanesulfonate (1.01 g, 2.50 mmol) in dry THF (20 mL) under nitrogen at −78° C. was added a solution of tert-butyllithium (1.7M in pentane) (2.94 mL, 5.00 mmol) via syringe over a period of 1 min. The resulting brown solution was stirred at −78° C. for 10 min, then N-fluoro-N-(phenylsulfonyl)benzenesulfonamide (0.867 g, 2.75 mmol) (recrystallized from diethyl ether prior to use) was added in one portion. The brown mixture was allowed to warm slowly to ambient temperature with stirring for 16 h. The reaction mixture was diluted with water, brine, and ethyl acetate, then the phases were partitioned. The organic phase was further washed with water and brine, then dried (MgSO₄), filtered, concentrated in vacuo, and the brown residue partially purified by flash chromatography on silica, eluting with hexane/toluene (9:1). The impure fractions were combined and further purified by HPLC (Kromasil Si60 silica gel column), eluting with hexane/acetone (98:2), to yield the title intermediate as a colourless oil.

### d) (2R)-4-(5-Fluoro-2-naphthyl)-2-methyl-1-(trifluoroacetyl)piperazine

To a solution of (2R)-methyl-1-trifluoroacetylpiperazine (0.21 g, 1.07 mmol) in dry toluene (5 mL) was added palladium acetate (10 mg, 0.045 mmol), (±)-2,2′-bis(diphenylphosphino)-1,1′-binaphthyl (39 mg, 0.063 mmol), cesium carbonate (0.407 g, 1.25 mmol), and then 5-fluoro-2-naphthyl trifluoromethanesulfonate (0.263 mg, 0.894 mmol) as a solution in toluene (5 mL) via cannula. The resultant mixture was degassed with three vacuum evacuation/nitrogen purge cycles, then heated to reflux under nitrogen overnight with stirring. The mixture was allowed to cool, filtered through a plug of celite, rinsing with ethyl acetate, and the filtrate washed with brine/water. The organic phase was dried (MgSO₄), filtered, concentrated in vacuo, and the oily brown residue purified by flash chromatography on silica, eluting with hexane/ethyl acetate (7:1), to yield the title intermediate as an off-white solid.

### e) (3R)-1-(5-Fluoro-2-naphthyl)-3-methylpiperazine

To a stirred solution of (2R)-4-(5-fluoro-2-naphthyl)-2-methyl-1-(trifluoroacetyl)piperazine (69 mg, 0.203 mmol) in dry methanol (4 mL) under nitrogen at ambient temperature was added sodium borohydride (30.7 mg, 0.812 mmol.) and the mixture stirred for 1 h. Two additional portions of sodium borohydride (2×30 mg) were added at 1 h intervals and stirring continued for another 1 h. The resultant pale green mixture was concentrated to a pale green solid, water added, and the product extracted into ethyl acetate. The combined organic extracts were washed with brine, then dried (MgSO₄), filtered, concentrated in vacuo, and the residue purified by flash chromatography on silica, eluting with dichloromethane/methanol (95:5, then 88:12) to yield the title intermediate as a tan solid.

### f) (1S)-1-{2-[(2R)-4-(5-Fluoro-2-naphthyl)-2-methylpiperazinyl]ethyl}-3,4-dihydro-1H-2-benzopyran-6-carboxamide

(3R)-1-(5-Fluoro-2-naphthyl)-3-methylpiperazine was coupled with (2S)-2-[6-(aminocarbonyl)-3,4-dihydro-1H-2-benzopyran-1-yl]ethyl methanesulfonate as described for Example 58 d) to yield the title compound as an amorphous tan solid. M+H=448.

### EXAMPLE 80

### (1S)-1-{2-[(2R)-4-(8-Fluoro-2-naphthyl)-2-methylpiperazinyl]ethyl}-3,4-dihydro-1H-2-benzopyran-6-carboxamide

Prepared as described above for Example 79, substituting 5-amino-2-naphthol with 8-amino-2-naphthol, to yield (1S)-1-{2-[(2R)-4-(8-fluoro-2-naphthyl)-2-methylpiperazinyl]ethyl}-3,4-dihydro-1H-2-benzopyran-6-carboxamide. M+H=448.

### EXAMPLE 81

### (1S)-1-{2-[(2R)-4-(5-Chloro-1-naphthyl)-2-methylpiperazinyl]ethyl}-3,4-dihydro-1H-2-benzopyran-6-carboxamide

### a) 5-Amino-1-naphthyl trifluoromethanesulfonate

Prepared from 5-amino-1-naphthol as described for Example 77 d).

### b) 5-Chloro-1-naphthyl trifluoromethanesulfonate

5-Amino-1-naphthyl trifluoromethanesulfonate (0.48 g, 1.6 mmol) was suspended in 37% hydrochloric acid (8 mL) and water (2 mL), and the suspension sonicated for 5 min before cooling to 0° C. A solution of sodium nitrite (0.171 g, 2.5 mmol) in water (2 mL) was added dropwise, maintaining the reaction temperature below 5° C. The reaction was stirred at 0° C. for 1.5 h, before adding dropwise to a solution of copper (I) chloride (0.816 g, 8.2 mmol) in water (4 mL) cooled to 0° C. The reaction was stirred at 0° C. for 30 min and at room temperature overnight. The reaction mixture was partitioned between water and diethyl ether, the organic layer washed with brine, dried (MgSO₄), filtered and concentrated in vacuo. The crude product was purified by column chromatography on silica, eluting with hexane, to yield the title compound as a colourless oil.

### c) (2R)-4-(5-Chloro-2-naphthyl)-2-methyl-1-(trifluoroacetyl)piperazine

Prepared from 5-chloro-1-naphthyl trifluoromethanesulfonate and (2R)-2-methyl-1-trifluoroacetylpiperazine as described for Example 79 d).

### d) (3R)-1-(5-Chloro-1-naphthyl)-3-methylpiperazine

Prepared from (2R)-4-(5-chloro-2-naphthyl)-2-methyl-1-(trifluoroacetyl)-piperazine by deprotection with sodium borohydride, as described for Example 79 e).

### e) (1S)-1-{2-[(2R)-4-(5-Chloro-i-naphthyl)-2-methylpiperazinyl]ethyl}-3,4-dihydro-1H-2-benzopyran-6-carboxamide

(3R)-1-(5-Chloro-1-naphthyl)-3-methylpiperazine was reacted with (1S)-2-[6-aminocarbonyl)-3,4-dihydro-1H-2-benzopyran-1-yl]ethyl methanesulfonate as described for Example 58 d), to yield (1S)-1-{2-[(2R)-4-(5-chloro-1-naphthyl)-2-methylpiperazinyl]ethyl}-3,4-dihydro-1H-2-benzopyran-6-carboxamide. M+H=464.

The following examples were prepared by substituting 5-chloro-1-naphthol in the above example with alternatively substituted chloronaphthols:

### EXAMPLE 82

### (1S)-1-{2-[(2R)-4-(6-Chloro-2-naphthyl)-2-methylpiperazinyl]ethyl}-3,4-dihydro-H-2-benzopyran-6-carboxamide

Prepared from 6-chloro-2-naphthol. M+H=464.

### EXAMPLE 83

### (1S)-1-{2-[(2R)-4-(7-Chloro-2-naphthyl)-2-methylpiperazinyl]ethyl)-3,4-dihydro-1H-2-benzopyran-6-carboxamide

Prepared from 7-chloro-2-naphthol. M+H=464.

### EXAMPLE 84

### (1S)-1-{2-[(2R)-4-(8-Chloro-2-naphthyl)-2-methylpiperazinyl]ethyl}-3,4-dihydro-1H-2-benzopyran-6-carboxamide

Prepared from 8-chloro-2-naphthol. M+H=464.

### EXAMPLE 85

### (1S)-1-[2-[4-(8-Chloro-2-naphthyl)piperazinyl]ethyl}-3,4-dihydro-1H-2-benzopyran-6-carboxamide

Prepared as described for Example 81, substituting piperazine for (2R)-methylpiperazine. M+H=450.

### EXAMPLE 86

### (1S)-1-{2-[(2R)-4-(7-Cyano-2-naphthyl)-2-methylpiperazinyl]ethyl}-3,4-dihydro-1H-2-benzopyran-6-carboxamide

### a) 7-Methoxy-2-naphthyl trifluoromethanesulfonate

Prepared from 7-methoxy-2-naphthol as described for Example 77 d).

### b) 7-Hydroxy-2-naphthyl trifluoromethanesulfonate

7-Methoxy-2-naphthyl trifluoromethanesulfonate (1.08 g, 3.5 mmol) was dissolved in dry dichloromethane (35 mL) under nitrogen, cooled to −78° C., and stirred as boron tribromide (1M in dichloromethane) (4.2 mL, 4.2 mmol) was added dropwise. The cooling bath was removed and the reaction stirred at room temperature overnight. Water was added slowly and the layers separated. The aqueous layer was extracted with dichloromethane. The combined organic layers were washed with brine, dried (MgSO₄), filtered and concentrated in vacuo to yield the title compound.

### c) 7-Hydroxy-2-naphthonitrile

7-Hydroxy-2-naphthyl trifluoromethanesulfonate (1.05 g, 3.5 mmol), zinc cyanide (0.844 g, 7 mmol) and tetrakis(triphenylphosphine)palladium(0) (0.166 g, 0.14 mmol) were placed in a round-bottom flask provided with a condenser under nitrogen, and dry DMF (17 mL) added. The mixture was degassed by three cycles of vacuum-nitrogen and heated at 120° C. for 2 h. The reaction mixture was allowed to cool to room temperature and partitioned between ethyl acetate and water. The aqueous layer was further extracted with ethyl acetate. The combined organic layers were washed with water and brine, dried (MgSO₄), filtered and concentrated in vacuo. The residue was purified by column chromatography to yield the title compound as a white solid.

### d) 7-Cyano-2-naphthyl trifluoromethanesulfonate

Prepared from 7-hydroxy-2-naphthonitrile as described for Example 77 d).

### e) (2R)-4-(7-Cyano-2-naphthyl)-2-methyl-1-(trifluoroacetyl)piperazine

Prepared from 7-cyano-2-naphthyl trifluoromethanesulfonate and (2R)-2-methyl-1-trifluoroacetylpiperazine as described for Example 79 d).

### f) (3R)-1-(7-Cyano-2-naphthyl)-3-methylpiperazine

To a solution of (2R)-4-(7-cyano-2-naphthyl)-2-methyl-1-(trifluoroacetyl)piperazine (0.473 g, 1.4 mmol) in methanol (15 mL) was added sodium borohydride (0.206 g, 5.5 mmol) in one portion and the reaction stirred at room temperature until the starting material was consumed as shown by TLC (2-4 h). The solvent was removed in vacuo and the residue partitioned between water and ethyl acetate. The aqueous layer was extracted with ethyl acetate, and the combined organic layers washed with brine, dried (MgSO₄), filtered and concentrated in vacuo. The residue was purified by column chromatography on silica, eluting with ethyl acetate/triethylamine (95:5), to yield the title compound as a yellow solid.

### g) (1S)-1-{2-[(2R)-4-(7-Cyano-2-naphthyl)-2-methylpiperazinyl]ethyl}-3,4-dihydro-1H-2-benzopyran-6-carboxamide

(3R)-1-(7-cyano-2-naphthyl)-3-methylpiperazine was reacted with (1S)-2-[6-aminocarbonyl)-3,4-dihydro-1H-2-benzopyran-1-yl]ethyl methanesulfonate as described for Example 58 d), to yield (1S)-1-{2-[(2R)-4-(7-cyano-2-naphthyl)-2-methylpiperazinyl]ethyl}-3,4-dihydro-1H-2-benzopyran-6-carboxamide. M+H=455.

### EXAMPLE 87

### (1S)-1-{2-[(2R)-4-(5-Cyano-2-naphthyl)-2-methylpiperazinyl]ethyl}-3,4-dihydro-1H-2-benzopyran-6-carboxamide

### a) 5-Cyano-2-naphthyl trifluoromethanesulfonate

To a stirred solution of 5-iodo-2-naphthyl trifluoromethanesulfonate (0.569 g, 1.415 mmol) in dry THF (15 mL) under nitrogen at ambient temperature was added copper (I) iodide (26.9 mg, 0.142 mmol), tetrakis(triphenylphosphine)palladium(0) (98.1 mg, 0.085 mmol), and sodium cyanide (139 mg, 2.83 mmol). The resultant mixture was heated under reflux for 5 ½ h. The mixture was allowed to cool to ambient temperature, diluted with ethyl acetate and washed with brine/water. The organic phase was dried (MgSO₄), filtered, concentrated in vacuo, and the residue purified by flash chromatography on silica, eluting with hexane/ethyl acetate (5:1), to yield the title intermediate as a white solid.

### b) (2R)-4-(5-Cyano-2-naphthyl)-2-methyl-1-(trifluoroacetyl)piperazine

To a solution of (2R)-2-methyl-1-trifluoroacetylpiperazine (257 mg, 1.313 mmol) in dry toluene (5 mL) was added palladium acetate (12.3 mg, 0.055 mmol), (±)-BINAP (47.7 mg, 0.077 mmol), cesium carbonate (499 mg, 1.532 mmol), and then 5-cyano-2-naphthyl trifluoromethanesulfonate (329 mg, 1.09 mmol) as a solution in toluene (5 mL) via cannula. The resultant mixture was degassed with three vacuum evacuation/N₂ purge cycles, then heated to reflux under nitrogen overnight with stirring. The mixture was allowed to cool, filtered through a plug of celite while rinsing with ethyl acetate (˜150 mL), and the filtrate washed with brine/water. The organic phase was dried (MgSO₄), filtered, concentrated, and the oily brown residue purified by flash chromatography on silica gel, eluting with hexanes/ethyl acetate (6:1, one step gradient to 5:1), to yield the title intermediate as a pale yellow solid.

### c) (3R)-1-(5-Cyano-2-naphthyl)-3-methylpiperazine

To a stirred pale yellow solution of (2R)-4-(5-cyano-2-naphthyl)7-2-methyl-1-(trifluoroacetyl)piperazine (283 mg, 0.816 mmol) in dry methanol (15 mL) under nitrogen at ambient temperature was added sodium borohydride (77 mg, 2.04 mmol) and the mixture stirred for 2 h. Two additional portions of sodium borohydride (50 mg each) were added at 1 h intervals, followed by stirring for an additional 1 h. The resultant opaque pale yellow solution was concentrated and the residue partitioned between water and ethyl acetate. The aqueous phase was further extracted withethyl acetate. The combined organic extracts washed with brine, dried (MgSO₄), filtered, concentrated, and the residue purified by flash chromatography on silica gel, eluting with dichloromethane/methanol (95:5, one step gradient to 9:1), to yield the title intermediate as a thick pale yellow oil.

### d) (1S)-1-{2-[(2R)-4-(5-Cyano-2-naphthyl)-2-methylpiperazinyl]ethyl}-3,4-dihydro-1H-2-benzopyran-6-carboxamide

(2R)-4-(5-Cyano-2-naphthyl)-2-methylpiperazine (185 mg, 0.735 mmol), potassium carbonate (305 mg, 2.205 mmol), potassium iodide (122 mg, 0.735 mmol), and (1S)-2-[6-aminocarbonyl)-3,4-dihydro-1H-2-benzopyran-1-yl]ethyl methane-sulfonate (242 mg, 0.809 mmol) were suspended in dry acetonitrile (10 mL) under nitrogen and heated to reflux overnight with stirring. The pale yellow mixture was allowed to cool to ambient temperature, then partitioned between ethyl acetate and water. The organic phase was further washed with water and brine, then dried (MgSO₄), filtered, concentrated, and the pale yellow oily residue partially purified by flash chromatography on silica gel, eluting with dichloromethane/methanol (95:5 one step gradient to 93:7). The combined impure fractions were further purified by flash chromatography on silica gel, eluting with ethyl acetate/methanol (9:1), to yield the title compound as a pale yellow solid. M+H=455.

### EXAMPLE 88

### (1S)-1-{2-[(2R)-4-(5-Cyano-1-benzothien-3-yl)-2-methylpiperazinyl]ethyl}-3,4-dihydro-1H-2-benzopyran-6-carboxamide

### a) Ethyl 3-amino-5-formyl-1-benzothiophene-2-carboxylate

A mixture of ethyl thioglycolate (1.2 g, 10 mmol), 2-fluoro-5-formyl-benzonitrile (1.49 g, 10 mmol), and triethylamine (3.04 g, 30 mmol) in DMSO (10 mL) was heated at 100° C. under-nitrogen for 3 h. After cooling to room temperature, water (60 mL) was added with stirring to give a yellow precipitate, which was filtered and dried in vacuo at 50° C. to give ethyl 3-amino-5-formyl-1-benzothiophene-2-carboxylate.

### b) Ethyl 3-amino-5-[(E)-(hydroxyimino)methyl]-1-benzothiophene-2-carboxylate

To an ice-cold solution of hydroxylamine hydrochloride (0.31 g, 4.46 mmol) in anhydrous acetonitrile (40 mL) was added triethylamine (0.45 g, 4.45 mmol) and ethyl 3-amino-5-formyl-1-benzothiophene-2-carboxylate (1 g, 4.021 mmol). The mixture was heated under reflux for 3 h under nitrogen. On cooling, ethyl 3-amino-5-[(E)-(hydroxyimino)methyl]-1-benzothiophene-2-carboxylate was given as a yellow solid.

### c) Ethyl 3-amino-5-cyano-1-benzothiophene-2-carboxylate

Trifluoroacetic anhydride (0.32 g, 1.5 mmol) was added to a mixture of ethyl 3-amino-5-[(E)-(hydroxyimino)methyl3-1-benzothiophene-2-carboxylate (0.4 g, 1.5 mmol) and triethylamine (0.34 g, 3.4 mmol) in acetonitrile (5 mL). After heating under ref lux for 1 day under nitrogen, additional triethylamine (0.64 g, 6.8 mmol) and trifluoroacetic anhydride (0.84 g, 4 mmol) were added and the suspension heated under reflux for a further 1 day. The solid was filtered off to give ethyl 3-amino-5-cyano-1-benzothiophene-2-carboxylate as a yellow solid.

### d) 3-[(3R)-3-Methyl-4-(trifluoroacetyl)piperazinyl]-1-benzothiophene-5-carbonitrile

Ethyl 3-amino-5-cyano-1-benzothiophene-2-carboxylate (0.1769, 0.715 mmol) and (R)-2-methyl-1-trifluoroacetylpiperazine (0.420 g, 2.14 mmol) were heated under nitrogen at 200° C. for four days. After cooling to room temperature, the black product was dissolved in a mixture of dichloromethane, ethanol and ethyl acetate. Excess water was added and the mixture extracted with ethyl acetate. The combined organic extracts were dried (MgSO₄) and evaporated to give a black oil. The oil was purified by flash chromatography on silica, eluting with cyclohexane/ethyl acetate (4:1), to yield 3-[(3R)-3-methyl-4-(trifluoroacetyl)piperazinyl]-1-benzothiophene-5-carbo-nitrile as an oil.

### e) 3-[(3R)-3-Methylpiperazinyl)-1-benzothiophene-5-carbonitrile

Sodium borohydride (83 mg, 2.16 mmol) was added in two portions over 20 min to a solution of 3-[(3R)-3-methyl-4-(trifluoroacetyl)piperazinyl]-1-benzothiophene-5-carbonitrile (83 mg, 0.235 mmol) in ethanol (6 mL) under nitrogen. After stirring overnight, excess water was added and the product extracted into ethyl acetate. The combined organic extracts were dried (MgSO₄) and evaporated to give 3-[(3R)-3-methylpiperazinyl]-1-benzothiophene-5-carbonitrile as a colourless oil.

### f) (1S)-1(2-[(2R)-4-(5-Cyano-1-benzothien-3-yl)-2-methylpiperazinyl]ethyl}-3,4-dihydro-1H-2-benzopyran-6-carboxamide

3-[(3R)-3-Methyl-4-(trifluoroacetyl)piperazinyl]-1-benzothiophene-5-carbonitrile was coupled with (1S)-2-[6-aminocarbonyl)-3,4-dihydro-1H-2-benzopyran-1-yl]ethyl methanesulfonate, as described for Example 58 d), to yield (1S)-1-{2-[(2R)-4-(5-cyano-1-benzothien-3-yl)-2-methylpiperazinyl]ethyl}-3,4-dihydro-1H-2-benzopyran-6-carboxamide. M+H=461.

### EXAMPLE 89

### (1S)-1-{2-[(2R)-4-(6-Cyano-1-benzothien-3-yl)-2-methylpiperazinyl]ethyl}-3,4-dihydro-1H-2-benzopyran-6-carboxamide

### a) 3-Bromo-1-benzothiophene-6-carbonitrile

To a solution of 1-benzothiophene-6-carbonitrile (2.13 g, 13.4 mmol) in dry DMF (20 mL) at −10° C. was added freshly recrystallised N-bromosuccinimide (2.38 g, 13.4 mmol). The solution was allowed to warm to room temperature and stirred over the weekend. The mixture was diluted with water and extracted into diethyl ether, and the organic extract washed with water, then brine. The extracts were dried (MgSO₄), filtered and evaporated in vacuo. The crude product was purified by flash chromatography on silica, eluting with ethyl acetate/hexane (1:9), to yield the title compound as a white solid.

### b) (3R)-1-(6-Cyano-1-benzothien-3-yl)-3-methylpiperazine

3-Bromo-1-benzothiophene-6-carbonitrile was coupled with (2R)-methylpiperazine, as described for Example 63 b), to yield (3R)-1-(6-cyano-1-benzothien-3-yl)-3-methylpiperazine.

### c) (1S)-1-{2-[(2R)-4-(6-Cyano-1-benzothien-3-yl)-2-methylpiperazinyl]ethyl}-3,4-dihydro-1H-2-benzopyran-6-carboxamide

(3R)-1-(6-Cyano-1-benzothien-3-yl)-3-methylpiperazine was coupled with (1S)-2-[6-aminocarbonyl)-3,4-dihydro-1H-2-benzopyran-1-yl]ethyl methane-sulfonate, as described for Example 58 d), to yield (1S)-1-(2-[(2R)-4-(6-cyano-1-benzothien-3-yl)-2-methylpiperazinyl]ethyl}-3,4-dihydro-1H-2-benzopyran-6-carboxamide. M+H=461.

### EXAMPLE 90

### (1S)-1-{2-[(2R)-4-(6-Cyano-1-benzothien-3-yl)-2-ethylpiperazinyl]ethyl}-3,4-dihydro-1H-2-benzopyran-6-carboxamide

Prepared as described for Example 89, substituting (2R)-methylpiperazine with (2R)-ethylpiperazine, to yield (1S)-1-{2-[(2R)-4-(6-cyano-1-benzothien-3-yl)-2-ethylpiperazinyl]ethyl}-3,4-dihydro-1H-2-benzopyran-6-carboxamide. M+H=475.

### EXAMPLE 91

### (1S)-1-{2-[(2S)-4-(6-Cyano-1-benzothien-3-yl)-2-ethylpiperazinyl]ethyl}-3,4-dihydro-1H-2-benzopyran-6-carboxamide

Prepared as described for-Example 89, substituting (2R)-methylpiperazine with (2S)-ethylpiperazine, to yield (1S)-1-{2-[(2S)-4-(6-cyano-1-benzothien-3-yl)-2-ethylpiperazinyl]ethyl}-3,4-dihydro-1H-2-benzopyran-6-carboxamide. M+H=475.

The following examples were prepared by substituting 6-cyano-1-benzothiophene in Example 89 with alternatively substituted benzothiophenes:

### EXAMPLE 92

### (1S)-1-{2-[(2R)-4-(6-Chloro-1-benzothien-3-yl)-2-methylpiperazinyl]ethyl}-3,4-dihydro-1H-2-benzopyran-6-carboxamide

Prepared from 6-chloro-1-benzothiophene. M+H=470.

### EXAMPLE 93

### (1S)-1-{2-[(2R)-4-(7-Fluoro-1-benzothien-3-yl)-2-methylpiperazinyl]ethyl}-3,4-dihydro-1H-2-benzopyran-6-carboxamide

Prepared from 7-fluoro-1-benzothiophene. M+H=454.

### EXAMPLE 94

### (1S)-1-{2-[(2R)-4-(7-Chloro-1-benzothien-3-yl)-2-methylpiperazinyl]ethyl}-3,4-dihydro-1H-2-benzopyran-6-carboxamide)

Prepared from 7-chloro-1-benzothiophene. M+H=470.

### EXAMPLE 95

### (1S)-1-{2-[(2R)-4-(6,7-Dichloro-1-benzothien-3-yl)-2-methylpiperazinyl]ethyl}-3,4-dihydro-1H-2-benzopyran-6-carboxamide

Prepared from 6,7-dichloro-1-benzothiophene. M+H=504.

The following examples were prepared as described for Example 89, substituting 3-bromo-1-benzothiophene-6-carbonitrile with alternative bromo-1-benzothiophenes:

### EXAMPLE 96

### (1S)-1-{2-[(2R)-4-(7-Fluoro-1-benzothien-4-yl)-2-methylpiperazinyl]ethyl}-3,4-dihydro-1H-2-benzopyran-6-carboxamide

Prepared from 4-bromo-7-fluoro-1-benzothiophene. M+H=454.

### EXAMPLE 97

### (1S)-1-{2-[(2R)-4-(1-Benzothien-5-yl)-2-methylpiperazinyl]ethyl}-3,4-dihydro-1H-2-benzopyran-6-carboxamide

Prepared from 5-bromo-1-benzothiophene. M+H=436.

### EXAMPLE 98

### (1S)-1-{2-[(2R)-4-(7-Methyl-1-benzothien-5-yl)-2-methylpiperazinyl]ethyl}-3,4-dihydro-1H-2-benzopyran-6-carboxamide

Prepared from 5-bromo-7-methyl-1-benzothiophene. M+H=450.

### EXAMPLE 99

### (1S)-1-{2-[(2R)-4-(7-Chloro-1-benzothien-5-yl)-2-methylpiperazinyl]ethyl}-3,4-dihydro-1H-2-benzopyran-6-carboxamide

Prepared from 5-bromo-7-chloro-1-benzothiophene. M+H=470.

### EXAMPLE 100

### (1S)-1-{2-[(2R)-4-(3-Methyl-1-benzothien-5-yl)-2-methylpiperazinyl]ethyl}-3,4-dihydro-1H-2-benzopyran-6-carboxamide

Prepared from 5-bromo-3-methyl-1-benzothiophene. M+H=450.

### EXAMPLE 101

### (1S)-1-{2-[(2R)-4-(1-Benzothien-6-yl)-2-methylpiperazinyl]ethyl}-3,4-dihydro-1H-2-benzopyran-6-carboxamide

Prepared from 6-bromo-1-benzothiophene. M+H=436.

### EXAMPLE 102

### (1S)-1-{2-[(2R)-4-(4-Chloro-1-benzothien-6-yl)-2-methylpiperazinyl]ethyl}-3,4-dihydro-1H-2-benzopyran-6-carboxamide

Prepared from 6-bromo-4-chloro-1-benzothiophene. M+H=470.

### EXAMPLE 103

### (1S)-1-{2-[(2R)-4-(3-Methyl-1-benzothien-6-yl)-2-methylpiperazinyl]ethyl}-3,4-dihydro-1H-2-benzopyran-6-carboxamide

Prepared from 6-bromo-3-methyl-1-benzothiophene. M+H=450.

### EXAMPLE 104

### (1S)-1-{2-[(2R)-4-(Benzothien-6-yl)-2-ethylpiperazinyl]ethyl}-3,4-dihydro-1H-2-benzopyran-6-carboxamide

Prepared from 6-bromo-1-benzothiophene and (2R)-ethylpiperazine. M+H=450.

### EXAMPLE 105

### (1S)-1-{2-[(2R)-4-(2-Cyano-1-benzothien-7-yl)-2-methylpiperazinyl]ethyl}-3,4-dihydro-1H-2-benzopyran-6-carboxamide

### a) 7-Bromo-1-benzothiophene-2-carboxaldehyde

To a solution of diisopropylamine (0.7 mL, 4.93 mmol) in dry tetrahydrofuran (10 mL) under nitrogen at 0° C. was added n-butyllithium (1.6M in hexanes) (3.1 mL, 4.93 mmol) over 5 min. After 10 min, this solution was added to 7-bromo-1-benzothiophene (1.0 g, 4.69 mmol) in dry tetrahydrofuran (10 mL) at −78° C. The reaction mixture was maintained at this temperature for 1 h, then dimethylformamide (0.55 mL, 7.03 mmol) added dropwise. After 10 min, the reaction was quenched by addition of acetic acid (2 mL) and water (25 mL). The solution was extracted into diethyl ether, washed with water, dried (Na₂SO₄), filtered and evaporated in vacuo, to yield 7-bromo-1-benzothiophene-2-carbaldehyde as a white solid.

### b) 7-Bromo-1-benzothiophene-2-carbonitrile

To a solution of 7-bromo-1-benzothiophene-2-carbaldehyde (1.0 g, 4.15 mmol) in 30% aqueous ammonia (40 mL) and tetrahydrofuran (4 mL) at room temperature was added iodine (1.05 g, 4.15 mmol). The mixture was stirred at this temperature for 7 h, then quenched with 5% aqueous solution of sodium thiosulfate (5 mL) and extracted with diethyl ether. The organic extracts were dried (MgSO₄), filtered and evaporated in vacuo. The crude product was purified by column chromatography on silica, eluting with hexane/ethyl acetate (9:1), to yield 7-bromo-1-benzothiophene-2-carbonitrile as a yellow solid.

### c) (3R)-7-(3-Methylpiperazin-1-yl)-1-benzothiophene-2-carbonitrile

A mixture of (2R)-methylpiperazine (0.265 mg, 2.63 mmol), (R)-2,2′-bis(diphenylphosphino)-1,1′-binaphthyl (102 mg, 0.075 mmol), palladium (II) acetate (25 mg, 0.05 mmol), cesium carbonate (0.998 g, 1.4 mmol) and 7-bromo-1-benzothiophene-2-carbonitrile (0.522 g, 2.19 mmol) in toluene (10 mL) was heated at 110° C. for 20 h. After cooling, the mixture was filtered through a short celite pad, eluting with ethyl acetate. The filtrate was concentrated in vacuo and the crude product purified by flash column chromatography on silica, eluting with dichloromethane/methanol (9:1), to yield (3R)-7-(3-methylpiperazin-1-yl)-1-benzothiophene-2-carbonitrile as a brown oil.

### d) (1S)-1-{2-[(2R)-4-(2-Cyano-1-benzothien-7-yl)-2-methylpiperazinyl]ethyl}-3,4-dihydro-1H-2-benzopyran-6-carboxamide

(3R)-7-(3-Methylpiperazin-1-yl)-1-benzothiophene-2-carbonitrile was coupled with (1S)-2-[6-aminocarbonyl)-3,4-dihydro-1H-2-benzopyran-1-yl]ethyl methane-sulfonate, as described for Example 58 d), to yield (1S)-1-{2-[(2R)-4-(2-cyano-1-benzothien-7-yl)-2-methylpiperazinyl]ethyl}-3,4-dihydro-1H-2-benzopyran-6-carboxamide. M+H=461.

### EXAMPLE 106

### (1S)-1-{2-[(2R)-4-(2-Cyano-4-fluoro-1-benzothien-4-yl)-2-methylpiperazinyl]ethyl}-3,4-dihydro-1H-2-benzopyran-6-carboxamide

### a) 3-Bromo-2,6-difluorobenzaldehyde

To a solution of 1-bromo-2,4-difluorobenzene (1.36 g, 7 mmol) in dry THF (20 mL) at −78° C. under nitrogen was added lithium diisopropylamide (2M in THF) (3.5 mL, 7 mmol) and stirred for 1h. Dimethylformamide (0.545 mL, 7 mmol) was added and stirred at −78° C. for 30 min. The reaction was quenched with acetic acid and extracted from water into dichloromethane. The organic extracts were washed with 1M hydrochloric acid, aqueous sodium hydrogen carbonate and brine, dried (MgSO₄), filtered and evaporated in vacuo. The crude product was purified by flash chromatography on silica, eluting with ethyl acetate/hexane (3:17), to yield the title compound as a yellow solid.

### b) Ethyl 7-bromo-4-fluoro-1-benzothiophene-2-carboxylate

Ethyl thioglycolate (0.355 mL, 3.24 mmol) in acetonitrile (2 mL) was cooled to 0° C. and stirred as triethylamine (0.615 mL, 4.4 mmol) was added. This solution was then added to a stirred solution of 3-bromo-2,6-difluorobenzaldehyde (0.65 g, 2.94 g) in acetonitrile (10 mL) and DMSO (3 mL) at 0° C. under nitrogen. The reaction mixture was allowed to warm to room temperature and stirred for 18 h. The mixture was diluted with water and extracted into ethyl acetate. The organic extracts were washed with 1M hydrochloric acid, then brine, dried (MgSO₄), filtered and evaporated in vacuo. This yielded the title compound as a yellow solid, which was used in the next step without further purification.

### c) 7-Bromo-4-fluoro-1-benzothiophene-2-carboxylic acid

Ethyl 7-bromo-4-fluoro-1-benzothiophene-2-carboxylate (4.44 g, 15 mmol) in 2M aqueous sodium hydroxide (20 mL) and ethanol (3 mL) was heated under reflux for 2 h. The mixture was diluted with water, acidified with 2M hydrochloric acid and extracted into ethyl acetate. The combined organic extracts were washed with brine, dried (MgSO₄), filtered and evaporated in vacuo to yield the title compound as a yellow solid.

### d) 7-Bromo-4-fluoro-1-benzothiophene-2-carboxamide

To a solution of 7-bromo-4-fluoro-1-benzothiophene-2-carboxylic acid (0.425 g, 1.54 mmol) in dry THF (10 mL) under nitrogen was added carbonyl diimidazole (0.275 g, 1.7 mmol) and triethylamine (0.65 mL, 4.6 mmol). The solution was stirred for 1 h after which a white precipitate formed. Dichloromethane (5 mL) was added and stirring continued for 18 h. Ammonia gas was bubbled through the solution for 3 h. The reaction mixture was extracted from water into ethyl acetate, the combined organic extracts washed with aqueous sodium hydrogen carbonate, then brine, dried (MgSO₄), filtered and evaporated in vacuo, to yield the title compound as a white solid.

### e) 7-Bromo-4-fluoro-1-benzothiophene-2-carbonitrile

To a stirred solution of 7-bromo-4-fluoro-1-benzothiophene-2-carboxamide (0.2 g, 0.73 mmol) in dry pyridine (10 mL) at 0° C. was added methanesulfonyl chloride (1 mL) under nitrogen. The mixture was left to stir over the weekend, then poured into water, and the resultant precipitate filtered off. Drying in a vacuum oven yielded the title compound as a brown solid.

### f) (3R)-4-Fluoro-7-(3-methylpiperazin-1-yl)-1-benzothiophene-2-carbonitrile

7-Bromo-4-fluoro-1-benzothiophene-2-carbonitrile was coupled with (2R)-methyl piperazine as described for Example 63 b), to yield (3R)-4-fluoro-7-(3-methylpiperazin-1-yl)-1-benzothiophene-2-carbonitrile

### g) (1S)-1-{2-[(2R)-4-(2-Cyano-4-fluoro-1-benzothien-4-yl)-2-methylpiperazinyl]ethyl}-3,4-dihydro-1H-2-benzopyran-6-carboxamide

(3R)-4-Fluoro-7-(3-methylpiperazin-1-yl)-1-benzothiophene-2-carbonitrile was coupled with (1S)-2-[6-aminocarbonyl)-3,4-dihydro-1H-2-benzopyran-1-yl]ethyl methanesulfonate, as described for Example 1 c), to yield (1S)-1-{2-[(2R)-4-(2-cyano-4-fluoro-1-benzothien-4-yl)-2-methylpiperazinyl]ethyl}-3,4-dihydro-1H-2-benzopyran-6-carboxamide. M+H=479.

### EXAMPLE 107

### (1S)-1-{2-[(2R)-4-(2-Cyano-7-fluoro-1-benzothien-4-yl)-2-methylpiperazinyl]ethyl}-3,4-dihydro-1H-2-benzopyran-6-carboxamide

Prepared as described for Example 106, substituting 1-bromo-3,4-difluorobenzene for 1-bromo-2,4-difluorobenzene as starting material, to yield (1S)-1-{2-[(2R)-4-(2-Cyano-7-fluoro-1-benzothien-4-yl)-2-methylpiperazinyl]ethyl}-3,4-dihydro-1H-2-benzopyran-6-carboxamide. M+H=479.

### EXAMPLE 108

### (1S)-1-{2-[(2R)-4-(3-Chloro-1-benzothien-5-yl)-2-methylpiperazinyl]ethyl}-3,4-dihydro-1H-2-benzopyran-6-carboxamide

### a) 5-Bromo-2-trimethylsilyl-1-benzothiophene

To a solution of 5-bromo-1-benzothiophene (0.50 g, 2.35 mmol) in dry THF (15 mL) at −78° C. under nitrogen was added chlorotrimethylsilane (0.6 mL, 4.73 mmol). To the mixture was then added freshly prepared lithium diisopropylamide (2.6 mmol) in THF (6 mL), and the reaction stirred for 2 h at −78° C. The reaction was quenched by pouring onto saturated aqueous ammonium chloride and extracted into diethyl ether. The combined organic extracts were washed with brine, dried (MgSO₄), filtered and evaporated in vacuo to yield the title compound as a brown oil.

### b) 5-Bromo-3-chloro-2-trimethylsilyl-1-benzothiophene

To a solution of 5-bromo-2-trimethylsilyl-1-benzothiophene (0.35 g, 1.23 mmol) in dry DMF (10 mL) under nitrogen was added N-chlorosuccinimide (0.174 g, 1.30 mmol) and stirred at 75° C. for 3 h. After allowing to cool to room temperature, the DMF was removed by evaporation under high vacuum and the residue extracted from water into ethyl acetate. The combined organic extracts were washed with brine, dried (MgSO₄), filtered and evaporated in vacuo. The crude product was purified by filtration through a pad of silica, eluting with hexane, to yield the title compound as off-white needles.

### c) 5-Bromo-3-chloro-1-benzothiophene

To a solution of 5-bromo-3-chloro-2-trimethylsilyl-1-benzothiophene (0.37 g, 1.16 mmol) in THF (6 mL) was added tetrabutylammonium fluoride (1M in THF) (1.27 mL, 1.27 mmol) and the reaction stirred at room temperature for 3 h. The reaction was quenched by the addition of water and extracted with ethyl acetate. The combined organic extracts were washed with 0.5% hydrochloric acid, then brine, dried (MgSO₄), filtered and evaporated in vacuo. The crude product was purified by filtration through a pad of silica, eluting with ethyl acetate/hexane (1:9), to yield the title compound as a pink solid.

### d) (3R)-1-(3-Chloro-1-benzothien-5-yl)-3-methylpiperazine

5-Bromo-3-chloro-1-benzothiophene was coupled with (2R)-methylpiperazine, as described for Example 63 b), to yield (3R)-1-(3-chloro-1-benzothien-5-yl)-3-methylpiperazine.

### e) (1S)-1-{2-[(2R)-4-(3-Chloro-1-benzothien-5-yl)-2-methylpiperazinyl]ethyl}-3,4-dihydro-1H-2-benzopyran-6-carboxamide

(3R)-1-(3-Chloro-1-benzothien-5-yl)-3-methylpiperazine was coupled with (1S)-2-[6-aminocarbonyl)-3,4-dihydro-1H-2-benzopyran-1-yl]ethyl methanesulfonate, as described for Example 1 c), to yield (1S)-1-{2-[(2R)-4-(3-chloro-1-benzothien-5-yl)-2-methylpiperazinyl]ethyl}-3,4-dihydro-1H-2-benzopyran-6-carboxamide. M+H=470.

### EXAMPLE 109

### (1S)-1-{2-[(2R)-4-(3-Chloro-1-benzothien-6-yl)-2-methylpiperazinyl]ethyl}-3,4-dihydro-1H-2-benzopyran-6-carboxamide

Prepared as described for Example 108, substituting 6-bromo-1-benzothiophene for 5-bromo-1-benzothiophene as starting material, to yield (1S)-1-{2-[(2R)-4-(3-chloro-1-benzothien-6-yl)-2-methylpiperazinyl]ethyl}-3,4-dihydro-1H-2-benzopyran-6-carboxamide. M+H=470.

### EXAMPLE 110

### (1S)-1-{2-[(2R)-4-(3-Cyano-1-benzothien-6-yl)-2-methylpiperazinyl]ethyl}-3,4-dihydro-1H-2-benzopyran-6-carboxamide

### a) 6-Bromo-1-benzothiophene-3-carboxaldehyde

To a solution of 6-bromo-3-methyl-1-benzothiophene (2.0 g, 8.8 mmol) in carbon tetrachloride (50 mL) was added N-bromosuccinimide (3.14 g, 17.6 mmol) and the mixture heated under reflux for 2.5 h while irradiating with a 1 kW flood lamp. The reaction mixture was cooled to room temperature and the solvent removed in vacuo. The residue was taken up in toluene (40 mL), silica gel (20 g) added and the mixture heated under reflux for 18 h. After cooling to room temperature, the silica was removed by filtration, washing with additional toluene and the combined organic fractions evaporated in vacuo. The crude product was purified by flash chromatography on silica, eluting with ethyl acetate/petroleum ether (0:100 to 100:0), to yield the title compound as off-white crystals.

### b) 6-Bromo-1-benzothiophene-3-carbaldehyde oxime

A mixture of 6-bromo-1-benzothiophene-3-carboxaldehyde (0.76 g, 3.2 mmol) and hydroxylamine hydrochloride (0.66 g, 3.5 mmol) in water (1 mL), pyridine (6 mL) and ethanol (60 mL) was heated under reflux for 30 min. The reaction mixture was cooled and the solvent removed in vacuo. The residue was extracted from water into ethyl acetate, and the combined organic extracts washed with brine, dried (MgSO₄), filtered and evaporated. The crude product was purified by flash chromatography on silica, eluting with ethyl acetate/hexane (5:95 to 100:0), to yield the title compound as a mixture of geometric isomers (trans/cis approx. 9:1).

### c) 6-Bromo-1-benzothiophene-3-carbonitrile

To a solution of 6-bromo-1-benzothiophene-3-carbaldehyde oxime (0.67 g, 2.6 mmol) in dry chloroform (55 mL) and triethylamine (0.35 mL, 6.8 mmol), cooled to 0° C., was added methanesulfonyl chloride (0.284 mL, 3.67 mmol). The reaction was allowed to warm to room temperature and stirred for 5 h. The reaction was quenched by addition of water and extracted with ethyl acetate. The combined organic extracts were washed with 2M hydrochloric acid, then brine, dried (MgSO₄), filtered and evaporated in vacuo. The crude product was purified by flash chromatography on silica, eluting with ethyl acetate/hexane (1:9), to yield the title compound.

### d) (3R)-6-(3-Methylpiperazin-1-yl)-1-benzothiophene-3-carbonitrile

6-Bromo-1-benzothiophene-3-carbonitrile was coupled with (2R)-methylpiperazine, as described for Example 63 b), to yield (3R)-6-(3-methylpiperazin-1-yl)-1-benzothiophene-3-carbonitrile.

### e) (1S)-1-{2-[(2R)-4-(3-Cyano-1-benzothien-6-yl)-2-methylpiperazinyl]ethyl)-3,4-dihydro-1H-2-benzopyran-6-carboxamide

(3R)-6-(3-Methylpiperazin-1-yl)-1-benzothiophene-3-carbonitrile was coupled with (1S)-2-[6-aminocarbonyl)-3,4-dihydro-1H-2-benzopyran-1-yl]ethyl methane-sulfonate, as described for Example 1 c.), to yield (1S)-1-{2-[(2R)-4-(3-Cyano-1-benzothien-6-yl)-2-methylpiperazinyl]ethyl)-3,4-dihydro-1H-2-benzopyran-6-carboxamide. M+H=461.

### EXAMPLE 111

### (1S)-1-{2-[(2R)-4-(3-Cyano-1-benzothien-5-yl)-2-methylpiperazinyl]ethyl}-3,4-dihydro-1H-2-benzopyran-6-carboxamide

Prepared as described for Example 110, substituting 5-bromo-2-trimethylsilyl-1-benzothiophene for 6-bromo-2-trimethylsilyl-1-benzothiophene as starting material, to yield (1S)-1-{2-[(2R)-4-(3-cyano-1-benzothien-5-yl)-2-methylpiperazinyl]ethyl}-3,4-dihydro-1H-2-benzopyran-6-carboxamide. M+H=461.

### EXAMPLE 112

### (1S)-1-{2-[(2R)-4-(2-Chloro-1-benzothien-6-yl)-2-methylpiperazinyl]ethyl}-3,4-dihydro-1H-2-benzopyran-6-carboxamide

### a) tert-Butyl (2R)-4-(2-chloro-1-benzothien-6-yl)-2-methyl-1-piperazinecarboxylate

To a solution of tert-butyl (2R)-4-(1-benzothien-6-yl)-2-methyl-1-piperazinecarboxylate (1.0 g, 3 mmol) in dry THF (35 mL) at −78° C. under nitrogen was added tert-butyllithium (1.5M solution in pentane) (4 mL, 6 mmol). The solution was stirred for 1 h, then N-chlorosuccinimide (1.0 g, 7.5 mmol) in THF was added and stirred for an additional 1 h. The mixture was allowed to warm to room temperature and stirred overnight. The reaction was diluted with water and extracted into ethyl acetate. The combined organic extracts were washed with brine, dried (MgSO₄), filtered and evaporated in vacuo. The crude product was purified by flash chromatography on silica to yield the title compound.

### b) (3R)-1-(2-Chloro-1-benzothien-6-yl)-3-methylpiperazine

tert-Butyl (2R)-4-(2-chloro-1-benzothien-6-yl)-2-methyl-1-piperazine-carboxylate was deprotected with trifluoroacetic acid, as described for Example 62 d), to yield (3R)-1-(2-chloro-1-benzothien-6-yl)-3-methylpiperazine.

### c) (1S)-1-{2-[(2R)-4-(2-Chloro-1-benzothien-6-yl)-2-methylpiperazinyl]ethyl)-3,4-dihydro-1H-2-benzopyran-6-carboxamide

(3R)-1-(2-Chloro-1-benzothien-6-yl)-3-methylpiperazine was coupled with (1S)-2-[6-aminocarbonyl)-3,4-dihydro-1H-2-benzopyran-1-yl]ethyl methane-sulfonate, as described for Example 1 c), to yield (1s)-1-{2-[(2R)-4-(2-chloro-1-benzothien-6-yl)-2-methylpiperazinyl]ethyl}-3,4-dihydro-1H-2-benzopyran-6-carboxamide. M+H=470.

### EXAMPLE 113

### (1S)-1-{2-[(2R)-4-(5-Cyanothieno[3,2-b]thien-3-yl)-2-methylpiperazinyl]ethyl}-3,4-dihydro-1H-2-benzopyran-6-carboxamide

### a) 3,4-Dibromo-2-thiophenecarbaldehyde

To a solution of 3,4-dibromothiophene (5.77 g, 23.8 mmol) in dry tetrahydrofuran (50 mL) under nitrogen at −78° C. was added lithium diisopropylamide (2M in THF) (11.9 mL, 23.8 mmol) over 5 min. The reaction mixture was maintained at this temperature for 1 h, then dimethylformamide (1.85 mL, 23.8 mmol) added dropwise. After 10 min, the reaction was quenched by addition of acetic acid and water. The solution was extracted into diethyl ether, washed with water, dried (Na₂SO₄), filtered and evaporated in vacuo, to yield 3,4-dibromo-2-thiophenecarbaldehyde.

### b) Ethyl 6-bromothieno[3,2-b]thiophene-2-carboxylate

To a solution of 3,4-dibromo-2-thiophenecarbaldehyde (5.96 g, 22 mmol) in DMSO (14 mL) and acetonitrile (50 mL) at 0° C. was added ethyl thioglycolate (2.7 mL, 24 mmol) and triethylamine (4.6 mL) in acetonitrile (15 mL). The mixture was allowed to warm to room temperature and stirred overnight. The reaction was diluted with water and extracted into dichloromethane. The organic extracts were washed with water, dried (MgSO₄), filtered and evaporated in vacuo. The crude product was purified by flash chromatography on silica, eluting with ethyl acetate/hexane (1:9), to yield ethyl 6-bromothieno[3,2-b]thiophene-2-carboxylate.

### c) 6-Bromothieno[3,2-b]thiophene-2-carboxylic acid

Ethyl 6-bromothieno[3,2-b]thiophene-2-carboxylate (4.44 g, 15.3 mmol) in ethanol (3 mL) and 2M aqueous sodium hydroxide (20 mL) was heated under reflux for 2 h. The mixture was cooled to room temperature, acidified with 2M hydrochloric acid, and extracted into ethyl acetate. The combined organic extracts were washed with brine, dried (MgSO₄), filtered and evaporated in vacuo, to yield the title compound as a yellow solid.

### d) 6-Bromothieno[3,2-b]thiophene-2-carboxamide

To a solution of 6-bromothieno[3,2-b]thiophene-2-carboxylic acid (0.46 g, 1.75 mmol) in dry THF (10 mL) under nitrogen was added carbonyl diimidazole (0.31 g, 1.92 mmol) and triethylamine (0.73 mL, 5.2 mmol), and the resultant orange solution stirred overnight at room temperature. Ammonia gas was then bubbled through the reaction for 3 h. The mixture was diluted with water and extracted into ethyl acetate. The combined organic extracts were washed with brine, dried (MgSO₄), filtered and evaporated in vacuo, to yield the title compound as a tan-solid.

### e) 6-Bromothieno[3,2-b]thiophene-2-carbonitrile

6-Bromothieno[3,2-b]thiophene-2-carboxamide was reacted with methane-sulfonyl chloride, as described in Example 106 e), to yield the title compound.

### f) 6-[(3S)-3-Methylpiperazinyl]thieno[3,2-b]thiophene-2-carbonitrile

6-Bromothieno[3,2-b]thiophene-2-carbonitrile was coupled with (2R)-methylpiperazine, as described for Example 63 b), to yield 6-[(3S)-3-methylpiperazinyl]thieno[3,2-b]thiophene-2-carbonitrile.

### g) (1S)-1-(2-[(2R)-4-(5-Cyanothieno[3,2-b]thien-3-yl)-2-methylpiperazinyl]ethyl}-3,4-dihydro-1H-2-benzopyran-6-carboxamide

6-[(3S)-3-Methylpiperazinyl]thieno[3,2-b]thiophene-2-carbonitrile was coupled with (1S)-2-[6-aminocarbonyl)-3,4-dihydro-1H-2-benzopyran-1-yl]ethyl methanesulfonate, as described for Example 1 c), to yield (1S)-1-{2-[(2R)-4-(5-cyanothieno[3,2-b]thien-3-yl)-2-methylpiperazinyl]ethyl}-3,4-dihydro-1H-2-benzopyran-6-carboxamide. M+H=467.

### EXAMPLE 114

### (1s)-1-{2-[4-(6-Fluoro-1-naphthyl)-1-piperidinyl]ethyl}-3,4-dihydro-1H-2-benzopyran-6-carboxamide

### a) tert-Butyl 4-(4,4,5,5-tetramethyl-1,3,2-dioxaborolan-2-yl)-3,6-dihydro-1(2H)-pyridinecarboxylate

Synthesised according to the procedure described by Paul R. Eastwood in Tetrahedron Letters, 2000, 41, 3705-3708.

### b) tert-Butyl 4-(6-fluoro-1-naphthyl)-3,6-dihydro-1(2H)-pyridinecarboxylate

To a nitrogen flushed flask containing tert-butyl 4-(4,4,5,5-tetramethyl-1,3,2-dioxaborolan-2-yl)-3,6-dihydro-1(2H)-pyridinecarboxylate (0.10 g, 0.32 mmol), 6-fluoro-1-naphthyl trifluoromethanesulfonate (0.10 g, 0.34 mmol), potassium carbonate (0.13 g, 0.97 mmol) and 1,1′-bis(diphenylphosphino)ferrocenedichloropalladium(II) (0.016 mg, 0.02 mmol) was added dry DMF (3 mL). The flask was evacuated and flushed with nitrogen three times. The mixture was heated to 80° C. and stirred overnight at this temperature. Purification of the reaction mixture by flash chromatography on silica, eluting with ethyl acetate/cyclohexane (1:10), yielded tert-butyl 4-(6-fluoro-1-naphthyl)-3,6-dihydro-1(2H)-pyridinecarboxylate as a colourless oil.

### c) tert-Butyl 4-(6-fluoro-1-naphthyl)-1-piperidinecarboxylate

To 10% palladium on carbon (0.14 g) under nitrogen was added methanol (1 mL). To this was added tert-butyl 4-(6-fluoro-1-naphthyl)-3,6-dihydro-1(2H)-pyridinecarboxylate (0.14 g, 0.43 mmol) in methanol (2 mL) followed by ammonium formate (1.08 g, 17.2 mmol). The mixture was heated for 2 days at 65° C. After this time, the catalyst was removed by filtration through celite. The celite was washed with methanol. The resulting solution was concentrated in vacuo to give a solid, which was dissolved in ethyl acetate. This solution was washed with water, dried (MgSO₄) and concentrated in vacuo to yield tert-butyl 4-(6-fluoro-1-naphthyl)-1-piperidinecarboxylate as an off-white solid.

### d) 4-(6-Fluoro-1-naphthyl)piperidine

To a suspension of tert-butyl 4-(6-fluoro-1-naphthyl)-1-piperidinecarboxylate (0.14 g, 0.42 mmol) in dichloromethane (2 mL), at 0° C., was added trifluoroacetic acid (0.097 g, 0.85 mmol). After stirring for 1 hour at 0° C. additional trifluoroacetic acid (0.45 mL) was added. The reaction mixture was allowed to warm to room temperature for 1.5 hours. The reaction mixture was then poured into 2N sodium hydroxide solution (50 mL) and extracted with dichloromethane. The combined organic extracts were dried (MgSO₄) and concentrated in vacuo to yield 1-(6-fluoro-1-naphthyl)piperidine as a pale brown coloured oil.

### e) (1S)-1-{2-[6-Fluoro-1-naphthyl)-1-piperidinyl]ethyl}-3,4-dihydro-1H-2-benzopyran-6-carboxamide

To 1-(6-fluoro-1-naphthyl)piperidine (0.070 g, 0.31 mmol) in dry acetonitrile (10 mL) was added (1S)-2-[6-aminocarbonyl)-3,4-dihydro-1H-2-benzopyran-1-yl]ethyl methanesulfonate (0.091 g, 0.305 mmol), potassium carbonate (0.065 g, 0.46 mmol) and potassium iodide (0.051 g, 0.31 mmol). The mixture was heated under reflux overnight. Solids were then removed by filtration and washed with methanol. The resulting solution was adsorbed onto silica and purified by flash chromatography on silica. Elution with methanol/ethyl acetate (1:5) yielded (1S)-1-{2-[4-(6-fluoro-1-naphthyl)-1-piperidinyl]ethyl}-3,4-dihydro-1H-2-benzopyran-6-carboxamide as a pale yellow foam. M+H=433.

### EXAMPLE 115

### (1S)-1-{2-[4-(6-Cyano-1-naphthyl)-1-piperidinyl]ethyl}-3,4-dihydro-1H-2-benzopyran-6-carboxamide

### a) 5-Bromo-2-naphthamide

5-Bromo-2-naphthoic acid (2.5 g, 10 mmol) in thionyl chloride (20 mL) was heated under reflux for 4 h. The excess thionyl chloride was removed by evaporation in vacuo and the residue taken up in THF (20 mL). The resultant solution was cooled to 0° C. and stirred as 0.5M ammonia in dioxan (50 mL) was added over 5 min. The reaction mixture was allowed to warm to room temperature and stirred for 2 h. The mixture was diluted with ethyl acetate and washed with water, then brine. The organic extracts were dried (MgSO₄), filtered and evaporated in vacuo. The crude product was recrystallised from ethyl acetate/hexane to yield 5-bromo-2-naphthamide as a white solid.

### b) tert-Butyl 4-[6-(aminocarbonyl)-1-naphthyl]-3,6-dihydro-1(2H)-pyridinecarboxylate

To a nitrogen flushed flask containing tert-butyl 4-(4,4,5,5-tetramethyl-1,3,2-dioxaborolan-2-yl)-3,6-dihydro-1(2H)-pyridinecarboxylate (0.208 g, 0.67 mmol), 5-bromo-2-naphthamide (0.177 g, 0.71 mmol), potassium carbonate (0.28 g, 2.02 mmol) and 1,1′-bis(diphenylphosphino)ferrocenedichloropalladium(II) (0.033 mg, 0.04 mmol) was added dry DMF (5 mL).The mixture was then stirred overnight at 70-80° C. Water (50 mL) was added to the reaction mixture and extracted with diethyl ether. The combined organic extracts were dried (Na₂SO₄) and concentrated in vacuo to give a brown oil. The oil was purified by flash chromatography on silica, eluting with an ethyl acetate/hexane gradient (1:4 to 3:4), to yield tert-butyl 4-[6-(aminocarbonyl)-1-naphthyl]-3,6-dihydro-1(2H)-pyridinecarboxylate as a yellow oil.

### c) tert-Butyl 4-[6-(aminocarbonyl)-1-naphthyl]-1-piperidinecarboxylate

To 10% palladium on carbon (0.10 g) under nitrogen was added a solution of tert-butyl 4-[6-(aminocarbonyl)-1-naphthyl]-3,6-dihydro-1(2H)-pyridinecarboxylate (0.196 g, 0.56 mmol) in methanol (50 mL). The solution was hydrogenated overnight at 60 psi. The catalyst was removed by filtration through celite and solvent removed in vacuo to give a pale orange oil. The oil was purified by flash chromatography on silica, eluting with ethyl acetate/hexane (3:2), to yield tert-butyl 4-[6-(aminocarbonyl)-1-naphthyl]-1-piperidinecarboxylate as a colourless glass.

### c) tert-Butyl 4-(6-cyano-1-naphthyl)-1-piperidinecarboxylate

To a solution of tert-butyl 4-[6-(aminocarbonyl)-1-naphthyl]-1-piperidinecarboxylate (0.103 g, 0.29 mmol) in pyridine (4 mL) was added, at 0° C., methanesulfonyl chloride (0.187 mL, 2.42 mmol). The mixture was left to stir at room temperature over the weekend. The reaction mixture was then poured into water (100 ml) and extracted with ethyl acetate. The combined organic extracts were washed with brine, dried (Na₂SO₄) and concentrated in vacuo to give an orange oil. The compound was dissolved in methanol and washed through an SCX-2 column with further methanol. Concentration of these washings in vacuo yielded tert-butyl 4-(6-cyano-1-naphthyl)-1-piperidinecarboxylate as a yellow oil.

### d) 5-(4-Piperidinyl)-2-naphthonitrile

To a solution of tert-butyl 4-(6-cyano-1-naphthyl)-1-piperidinecarboxylate (0.079 g, 0.23 mmol) in dry dichloromethane (0.5 mL) at 0° C. was added trifluoroacetic acid (0.035 mL, 0.47 mmol). The mixture was then allowed to stir overnight at room temperature. To the reaction mixture was added ice (10 g) and this mixture made basic to pH 10-11 with potassium carbonate. The mixture was extracted with dichloromethane, and the combined organic extracts dried (Na₂SO₄) and concentrated in vacuo to yield an orange oil. The oil was taken up in methanol and loaded onto an SCX-2 column. The column was washed with methanol, then basic material eluted with a 2N solution of ammonia in methanol. Concentration in vacuo of the ammonia solution gave 5-(4-piperidinyl)-2-naphthonitrile as an orange oil.

### e) (1S)-1-(2-[4-(6-Cyano-1-naphthyl)-1-piperidinyl]ethyl}-3,4-dihydro-1H-2-benzopyran-6-carboxamide

5-(4-Piperidinyl)-2-naphthonitrile was coupled with (1S)-2-[6-aminocarbonyl)-3,4-dihydro-1H-2-benzopyran-1-yl]ethyl methanesulfonate, as described for Experiment 58 d), to yield (1S)-1-{2-[4-(6-cyano-1-naphthyl)-1-piperidinyl]ethyl)-3,4-dihydro-1H-2-benzopyran-6-carboxamide as a white solid. M+H=440.

### EXAMPLE 116

### (1S)-1-{2-[(2R)-4-(6-Cyano-1-naphthyl)-2-methylpiperazinyl]ethyl)-N-methyl-3,4-dihydro-1H-2-benzopyran-6-carboxamide

Prepared as described for Example 17, coupling the intermediate (1S)-2-[6-(N-methylaminocarbonyl)-3,4-dihydro-1H-2-benzopyran-1-yl]ethyl methanesulfonate with (3R)-1-(6-cyano-1-naphthyl)-3-methylpiperazine. M+H=469.

### EXAMPLE 117

### (1S)-1-{2-[(2R)-4-(6-Cyano-1-naphthyl)-2-methylpiperazinyl]ethyl}-N,N-dimethyl-3,4-dihydro-1H-2-benzopyran-6-carboxamide

Prepared as described for Example 17, coupling the intermediate (1S)-2-[6-(N,N-dimethylaminocarbonyl)-3,4-dihydro-1H-2-benzopyran-1-yl]ethyl methane-sulfonate with (3R)-1-(6-cyano-1-naphthyl)-3-methylpiperazine. M+H=483.

### EXAMPLE 118

### (2R)-4-(6-Fluoro-1-naphthyl)-2-methyl-1-{2-[(1S)-6-(1-methyl-1H-1,2,4-triazol-3-yl)-3,4-dihydro-1H-2-benzopyran-1-yl]ethyl}piperazine

### a) (1S)—N-[(Z)-(Dimethylamino)methylidene)-1-{2-[(2R)-4-(6-fluoro-1-naphthyl)-2-methylpiperazinyl]ethyl}-3,4-dihydro-1H-2-benzopyran-6-carboxamide

Prepared from (1S)-1-{2-[(2R)-4-(6-fluoro-1-naphthyl)-2-methylpiperazinyl]-ethyl}-3,4-dihydro-1H-2-benzopyran-6-carboxamide, according to the method described for Example 16 a).

### b) (2R)-4-(6-Fluoro-1-naphthyl)-2-methyl-1-{2-[(1S)-6-(1-methyl-1H-1,2,4-triazol-3-yl)-3,4-dihydro-1H-2-benzopyran-1-yl]ethyl}piperazine

Prepared from (1S)—N-[(Z)-(dimethylamino)methylidene]-1-{2-[(2R)-4-(6-fluoro-1-naphthyl)-2-methylpiperazinyl]ethyl}-3,4-dihydro-1H-2-benzopyran-6-carboxamide, according to the method described for Example 16 b), to yield the title compound. M+H=486.

### EXAMPLE 119

### (2R)-4-(6-Cyano-1-naphthyl)-2-methyl-1-{2-[(1S)-6-(1H-1,2,4-triazol-3-yl)-3,4-dihydro-1H-2-benzopyran-1-yl]ethyl}piperazine

Prepared as described for Example 118, substituting hydrazine for methylhydrazine, to yield the title compound. M+H=472.

### EXAMPLE 120

### (2R)-4-(6-Cyano-1-naphthyl)-2-methyl-1-{2-[(1S)-6-(3-pyridinyl)-3,4-dihydro-1H-2-benzopyran-1-yl]ethyl}piperazine

### a) 3-[(1S)-1-(2-{[tert-Butyl(dimethyl)silyl]oxy}ethyl)-3,4-dihydro-1H-2-benzopyran-6-yl]pyridine

{2-[(1S)-6-Bromo-3,4-dihydro-1H-2-benzopyran-1-yl]ethoxy}(tert-butyl)dimethylsilane (0.362 g, 0.975 mmol) dissolved in dry toluene (15 mL) was degassed by alternate evacuation and flushing with nitrogen. 3-(1,3,2-Dioxaborinan-2-yl)pyridine (0.275 g, 1.69 mmol), tetrakis(triphenylphosphine) palladium (0.072 g, 0.07 mmol), potassium hydroxide (0.218 g, 3.9 mmol) and tetrabutylammonium bromide (0.17 g, 0.53 mmol) were added and the mixture heated under reflux under nitrogen for 18 h. The reaction was cooled, diluted with dichloromethane and filtered through celite. The filtrate was washed with water, dried (MgSO₄), filtered and evaporated in vacuo to give the title compound, which was used in the next step without further purification.

### b) 2-[(1S)-6-(3-Pyridinyl)-3,4-dihydro-1H-2-benzopyran-1-yl]ethanol

3-[(1S)-1-(2-{[tert-Butyl(dimethyl)silyl]oxy}ethyl)-3,4-dihydro-1H-2-benzopyran-6-yl]pyridine was deprotected with aqueous acetic acid, as described for Example 1 a), to yield 2-[(1S)-6-(3-pyridinyl)-3,4-dihydro-1H-2-benzopyran-1-yl]ethanol.

### c) 2-[(1S)-6-(3-Pyridinyl)-3,4-dihydro-1H-2-benzopyran-1-yl]ethyl methanesulfonate

2-[(1S)-6-(3-Pyridinyl)-3,4-dihydro-1H-2-benzopyran-1-yl]ethanol was reacted with methanesulfonyl chloride, as described for Example 1 b), to yield 2-[(1S)-6-(3-pyridinyl)-3,4-dihydro-1H-2-benzopyran-1-yl]ethyl methanesulfonate.

### c) (2R)-4-(6-Cyano-1-naphthyl)-2-methyl-1-{2-[(1S)-6-(3-pyridinyl)-3,4-dihydro-1H-2-benzopyran-1-yl]ethyl}piperazine

2-[(1S)-6-(3-Pyridinyl)-3,4-dihydro-1H-2-benzopyran-1-yl]ethyl methane-sulfonate was coupled with (3R)-1-(6-cyano-1-naphthyl)-3-methylpiperazine, as described for Example 58 d), to yield (2R)-4-(6-cyano-1-naphthyl)-2-methyl-1-{2-[(1S)-6-(3-pyridinyl)-3,4-dihydro-1H-2-benzopyran-1-yl]ethyl}piperazine. M+H=489.

### EXAMPLE 121

### (2R)-4-(6-Cyano-1-naphthyl)-2-methyl-1-{2-[(1S)-6-(4-pyridinyl)-3,4-dihydro-1H-2-benzopyran-1-yl]ethyl}piperazine

The title compound was prepared as described above for Example 120, from {2-[(1S)-6-bromo-3,4-dihydro-1H-2-benzopyran-1-yl]ethoxy}(tert-butyl)dimethyl-silane and 4-(4,4,5,5-tetramethyl-1,3,2-dioxaborolan-2-yl)pyridine. M+H=489.

### EXAMPLE 122

### (1S)-1-{2-[(2R)-4-(6-Fluoro-1-naphthyl)-2-methylpiperazinyl]ethyl}-3,4-dihydro-1H-2-benzopyran-6-sulfonamide

### a) (1S)-1-(2-{[tert-Butyl(dimethyl)silyl]oxy}ethyl)-3,4-dihydro-1H-2-benzopyran-6-sulfonamide

To a stirred solution of {2-[(1S)-6-bromo-3,4-dihydro-1H-2-benzopyran-1-yl]ethoxy}(tert-butyl)dimethylsilane (1.0 g, 2.7 mmol) in dry tetrahydrofuran (15 mL) under nitrogen at −78° C. was added n-butyllithium (1.6M solution in THF) (1.9 mL, 2.96 mmol). After 45 min, gaseous sulphur dioxide was bubbled through the solution for 20 min. The reaction mixture was allowed to warm to room temperature and stirring continued for 2 h. The solvent was removed in vacuo, the crude product dissolved in dichloromethane (15 mL) and N-chlorosuccinimide (380 mg, 2.83 mmol) added in a single portion. After 1 h the suspension was filtered through a pad of celite and the filtrated evaporated to dryness. The crude product was dissolved in dioxan (10 mL) and gaseous ammonia bubbled through the solution for 10 min. The reaction mixture was then stirred at room temperature for 16 h. The solvent was removed in vacuo and the residue purified by column chromatography on silica gel, eluting with dichloromethane/methanol (95:5), to yield (1S)-1-(2-{[tert-butyl(dimethyl)silyl]oxy}-ethyl)-3,4-dihydro-1H-2-benzopyran-6-sulfonamide as a yellow oil.

### b) (1S)-1-(2-Hydroxyethyl)-3,4-dihydro-1H-2-benzopyran-6-sulfonamide

To a stirred solution of (1S)-1-(2-{[tert-butyl(dimethyl)silyl]oxy}-ethyl)-3,4-dihydro-1H-2-benzopyran-6-sulfonamide (0.95 g, 2.56 mmol) in tetrahydrofuran (5 mL) was added tetrabutylammonium fluoride (1M solution in tetrahydrofuran) (2.8 mL, 2.8 mmol). The reaction mixture was stirred at room temperature for 3 h, then the solvent removed in vacuo. The residue was purified by column chromatography on silica gel, eluting with dichloromethane/methanol (9:1), to yield (1S)-1-(2-hydroxyethyl)-3,4-dihydro-1H-2-benzopyran-6-sulfonamide as a white solid.

### c) (1S)-2-[6-Aminosulfonyl)-3,4-dihydro-1H-2-benzopyran-1-yl]ethyl methanesulfonate

Prepared from (1S)-1-(2-hydroxyethyl)-3,4-dihydro-1H-2-benzopyran-6-sulfonamide, as described for Example 1 b).

### d) (1S)-1-{2-[(2R)-4-(6-Fluoro-1-naphthyl)-2-methylpiperazinyl]ethyl}-3,4-dihydro-1H-2-benzopyran-6-sulfonamide

(1S)-2-[6-Aminosulfonyl)-3,4-dihydro-1H-2-benzopyran-1-yl]ethyl methane-sulfonate was coupled with (3R)-1-(6-1-fluoro-1-naphthyl)-3-methylpiperazine, as described for Example 58 d), to yield the title compound. M+H=484.

The following examples were prepared from (1S)-2-[6-aminosulfonyl)-3,4-dihydro-1H-2-benzopyran-1-yl]ethyl methanesulfonate as described above for Example 131, substituting (3R)-1-(6-fluoro-1-naphthyl)-3-methylpiperazine with alternative 1-aryl-piperazines:

### EXAMPLE 123

### (1S)-1-{2-[(2R)-4-(6-Cyano-1-naphthyl)-2-methylpiperazinyl]ethyl}-3,4-dihydro-1H-2-benzopyran-6-sulfonamide

Prepared from (3R)-1-(6-cyano-1-naphthyl)-3-methylpiperazine. M+H=491.

### EXAMPLE 124

### (1S)-1-{2-[(2R)-4-(6-Fluoro-1-benzothien-3-yl)-2-methylpiperazinyl]ethyl}-3,4-dihydro-1H-2-benzopyran-6-sulfonamide

Prepared from (3R)-1-(6-fluoro-1-benzothien-3-yl)-3-methylpiperazine. M+H=490.

### EXAMPLE 125

### (1S)-1-{2-[(2R)-4-(6-Cyano-1-benzothien-3-yl)-2-methylpiperazinyl]ethyl}-3,4-dihydro-1H-2-benzopyran-6-sulfonamide

Prepared from (3R)-1-(6-cyano-1-benzothien-3-yl)-3-methylpiperazine. M+H=497.

### EXAMPLE 126

### (1S)-1-{2-[(2R)-4-(6-Fluoro-1-naphthyl)-2-methylpiperazinyl]ethyl}-N-methyl-3,4-dihydro-1H-2-benzopyran-6-sulfonamide

### a) (1s)-1-(2-{[tert-Butyl(dimethyl)silyl]oxy}ethyl)-N-methyl-3,4-dihydro-1H-2-benzopyran-6-sulfonamide

Prepared according to the procedure described for the synthesis of (1S)-1-(2-{[tert-butyl(dimethyl)silyl]oxy}ethyl)-3,4-dihydro-1H-2-benzopyran-6-sulfonamide in Example 122, using a 2 M solution of methylamine in tetrahydrofuran instead of gaseous ammonia, to yield the title compound as a yellow oil.

### b) (1S)-1-(2-Hydroxyethyl)-N-methyl-3,4-dihydro-1H-2-benzopyran-6-sulfonamide

Prepared from (1S)-1-(2-{[tert-butyl(dimethyl)silyl]oxy}ethyl)-N-methyl-3,4-dihydro-1H-2-benzopyran-6-sulfonamide, as described for Example 122 b).

### c) (1S)-2-[N-methyl-6-aminosulfonyl)-3,4-dihydro-1H-2-benzopyran-1-yl]ethyl methanesulfonate

Prepared from (1S)-1-(2-hydroxyethyl)-N-methyl-3,4-dihydro-1H-2-benzopyran-6-sulfonamide, as described for Example 122 c).

### e) (1S)-1-{2-[(2R)-4-(6-Fluoro-1-naphthyl)-2-methylpiperazinyl]ethyl}-N-methyl-3,4-dihydro-1H-2-benzopyran-6-sulfonamide

(1S)-2-[N-Methyl-6-aminosulfonyl)-3,4-dihydro-1H-2-benzopyran-1-yl]ethyl methanesulfonate was coupled with (3R)-1-(6-fluoro-1-naphthyl)-3-methylpiperazine, as described for Example 58 d), to yield the title compound. M+H=498.

The following examples were prepared from (1S)-2-[N-methyl-6-aminosulfonyl-3,4-dihydro-1H-2-benzopyran-1-yl]ethyl methanesulfonate as described above for Example 126, substituting (3R)-1-(6-fluoro-1-naphthyl)-3-methylpiperazine with alternative 1-aryl-piperazines:

### EXAMPLE 127

### (1S)-1-{2-[(2R)-4-(6-Cyano-1-naphthyl)-2-methylpiperazinyl]ethyl}-N-methyl-3,4-dihydro-1H-2-benzopyran-6-sulfonamide

Prepared from (3R)-1-(6-cyano-1-naphthyl)-3-methylpiperazine. M+H=505.

### EXAMPLE 128

### (1S)-1-{2-[(2R)-4-(6-Cyano-1-benzothien-3-yl)-2-methylpiperazinyl]ethyl}-N-methyl-3,4-dihydro-1H-2-benzopyran-6-sulfonamide

Prepared from (3R)-1-(6-cyano-1-benzothien-3-yl)-3-methylpiperazine. M+H=511.

### EXAMPLE 129

### (1S)-1-{2-[(2R)-4-(6-Fluoro-1-naphthyl)-2-methylpiperazinyl]ethyl}-N,N-dimethyl-3,4-dihydro-1H-2-benzopyran-6-sulfonamide

### a) (1S)-1-(2-{[tert-Butyl (dimethyl)silyl]oxy}ethyl)-N,N-dimethyl -3,4-dihydro-1H-2-benzopyran-6-sulfonamide

Prepared according to the procedure described for the synthesis of (1S)-1-(2-[tert-butyl(dimethyl)silyl]oxy}ethyl)-3,4-dihydro-1H-2-benzopyran-6-sulfonamide in Example 122, using a 2 M solution of dimethylamine in tetrahydrofuran instead of gaseous ammonia, to yield the title compound as a yellow oil.

### b) (1S)-1-(2-Hydroxyethyl)-N,N-dimethyl-3,4-dihydro-1H-2-benzopyran-6-sulfonamide

Prepared from (1S)-1-(2-{[tert-butyl(dimethyl)silyl]oxy}ethyl)-N,N-dimethyl-3,4-dihydro-1H-2-benzopyran-6-sulfonamide, as described for Example 122 b).

### c) (1S)-2-[N,N-Dimethyl-6-aminosulfonyl)-3,4-dihydro-1H-2-benzopyran-1-yl]ethyl methanesulfonate

Prepared from (1S)-1-(2-hydroxyethyl)-N,N-dimethyl-3,4-dihydro-1H-2-benzopyran-6-sulfonamide, as described for Example 122 c).

### d) (1S)-1-{2-[(2R)-4-(6-Fluoro-1-naphthyl)-2-methylpiperazinyl]ethyl}-N,N-dimethyl-3,4-dihydro-1H-2-benzopyran-6-sulfonamide

(1S)-2-[N,N-Dimethyl-6-aminosulfonyl)-3,4-dihydro-1H-2-benzopyran-1-yl]ethyl methanesulfonate was coupled with (3R)-1-(6-fluoro-1-naphthyl)-3-methylpiperazine, as described for Example 58 d), to yield the title compound. M+H=512.

The following examples were prepared from (1S)-2-[N,N-dimethyl-6-aminosulfonyl-3,4-dihydro-1H-2-benzopyran-1-yl]ethyl methanesulfonate as described above for Example 129, substituting (3R)-1-(6-fluoro-1-naphthyl)-3-methylpiperazine with alternative 1-aryl-piperazines:

### EXAMPLE 130

### (1S)-1-{2-[(2R)-4-(6-Cyano-1-naphthyl)-2-methylpiperazinyl]ethyl}-N,N-dimethyl-3,4-dihydro-1H-2-benzopyran-6-sulfonamide

Prepared from (3R)-1-(6-cyano-1-naphthyl)-3-methylpiperazine. M+H=519.

### EXAMPLE 131

### (1S)-1-{2-[(2R)-4-(6-Fluoro-1-benzothien-3-yl)-2-methylpiperazinyl]ethyl}-N,N-dimethyl-3,4-dihydro-1H-2-benzopyran-6-sulfonamide

Prepared from (3R)-1-(6-fluoro-1-benzothien-3-yl)-3-methylpiperazine. M+H=525.

### EXAMPLE 132

### (1S)-5-Methyl-1-{2-[(2R)-4-(6-fluoro-1-naphthyl)-2-methylpiperazinyl]ethyl}-3,4-dihydro-1H-2-benzopyran-6-carboxamide

### a) (1S)-1-(2-{[tert-Butyl(dimethyl)silyl]oxy}ethyl)-N-(1-methyl-1-phenylethyl)-3,4-dihydro-1H-2-benzopyran-6-carboxamide

A mixture of (1S)-1-(2-{[tert-butyl(dimethyl)silyl]oxy}ethyl)-3,4-dihydro-1H-2-benzopyran-6-carboxylic acid (1.98 g, 5.88 mmol), cumylamine (0.8 g, 5.9 mmol), bromo-tris-pyrrrolidino-phosphonium hexafluorophosphate (3.93 g, 8.81 mmol) and di-iso-propylethylamine (1.84 mL, 8.81 mmol) in dichloromethane (20 mL) was stirred at room temperature for 1 h. The solvent was removed in vacuo and the crude mixture purified by column chromatography on silica gel, eluting with dichloromethane/methanol (95:5), to yield (1S)-1-(2-{[tert-butyl(dimethyl)silyl]-oxy}ethyl)-N-(1-methyl-1-phenylethyl)-3,4-dihydro-1H-2-benzopyran-6-carboxamide as a white solid.

### b) (1S)-1-(2-Hydroxyethyl)-5-methyl-3,4-dihydro-1H-2-benzopyran-6-carboxamide and (1S)-1-(2-Hydroxyethyl)-7-methyl-3,4-dihydro-1H-2-benzopyran-6-carboxamide

To a solution of (1S)-1-(2-{[tert-butyl(dimethyl)silyl]oxy}ethyl)-N-(1-methyl-1-phenylethyl)-3,4-dihydro-1H-2-benzopyran-6-carboxamide (0.95 g, 2.09 mmol) and tetramethylethylenediamine (1.01 mL, 6.7 mmol) in dry tetrahydrofuran (20 mL) at −78° C. under nitrogen was added sec-BuLi (1.6M solution in THF) (5.15 mL, 8.24 mmol). After stirring for 2 h, iodomethane (0.29 mL, 4.6 mmol) was added and the reaction allowed to warm to room temperature. The reaction mixture was stirred for 2 h, then quenched with a saturated aqueous solution of ammonium chloride and extracted into dichloromethane. The organic extracts were dried (Na₂SO₄) and evaporated to dryness. The crude product was dissolved in trifluoroacetic acid (2 mL) and ethanedithiol (0.5 mL) added. The mixture was stirred at room temperature for 16 h, then evaporated to dryness. The residue was dissolved in methanol (5 mL) and potassium carbonate added. After stirring for 1 h, the solvent was removed in vacuo and the crude product purified by column chromatography on silica gel, eluting with dichloromethane/methanol (9:1), to yield a mixture of 5- and 7-methyl benzopyrans. This mixture was subsequently separated by HPLC on a Kromasil Si60 column (4.6×250 mm), eluting with dichloromethane/methanol (95:5), to yield (1S)-1-(2-hydroxyethyl)-5-methyl-3,4-dihydro-1H-2-benzopyran-6-carboxamide and (1S)-1-(2-hydroxyethyl)-7-methyl-3,4-dihydro-1H-2-benzopyran-6-carboxamide as white solids.

### c) (1S)-2-[6-Aminocarbonyl)-5-methyl-3,4-dihydro-1H-2-benzopyran-1-yl]ethyl methanesulfonate

(1S)-1-(2-Hydroxyethyl)-5-methyl-3,4-dihydro-1H-2-benzopyran-6-carboxamide was reacted with methanesulfonyl chloride, as described for Example 1 b), to yield (1S)-2-[6-aminocarbonyl)-5-methyl-3,4-dihydro-1H-2-benzopyran-1-yl]ethyl methanesulfonate.

### d) (1S)-5-Methyl-1-{2-[(2R)-4-(6-fluoro-1-naphthyl)-2-methylpiperazinyl]ethyl}-3,4-dihydro-1H-2-benzopyran-6-carboxamide

(1S)-2-[6-Aminocarbonyl)-5-methyl-3,4-dihydro-1H-2-benzopyran-1-yl]ethyl methanesulfonate was coupled with (3R)-1-(6-fluoro-1-naphthyl)-3-methylpiperazine, as described for Example 1 c), to yield (1S)-5-methyl-1-{2-[(2R)-4-(6-fluoro-1-naphthyl)-2-methylpiperazinyl]ethyl}-3,4-dihydro-1H-2-benzopyran-6-carboxamide. M+H=462.

### EXAMPLE 132

### (1S)-7-Methyl-1-{2-[(2R)-4-(6-fluoro-1-naphthyl)-2-methylpiperazinyl]ethyl}-3,4-dihydro-1H-2-benzopyran-6-carboxamide

Following the procedure described above for Example 131, (1S)-7-methyl-1-{2-[(2R)-4-(6-fluoro-1-naphthyl)-2-methylpiperazinyl]ethyl}-3,4-dihydro-1H-2-benzopyran-6-carboxamide was prepared from (1S)-1-(2-hydroxyethyl)-7-methyl-3,4-dihydro-1H-2-benzopyran-6-carboxamide (from Example 131 b)). M+H=462.

### EXAMPLE 133

### (1S)-7-Fluoro-1-{2-[(2R)-4-(6-fluoro-1-naphthyl)-2-methylpiperazinyl]ethyl}-3,4-dihydro-1H-2-benzopyran-6-carboxamide

### a) (1S)-1-(2-Hydroxyethyl)-7-fluoro-3,4-dihydro-1H-2-benzopyran-6-carboxamide

Following the procedure used for the synthesis of (1S)-1-(2-hydroxyethyl)-7-methyl-3,4-dihydro-1H-2-benzopyran-6-carboxamide and using N-fluorobenzenesulfonimide instead of iodomethane, the title compound (1S)-1-(2-hydroxyethyl)-7-fluoro-3,4-dihydro-1H-2-benzopyran-6-carboxamide was isolated as a white solid.

### b) (1S)-2-[6-Aminocarbonyl)-7-fluoro-3,4-dihydro-1H-2-benzopyran-1-yl]ethyl methanesulfonate

(1S)-1-(2-Hydroxyethyl)-7-fluoro-3,4-dihydro-1H-2-benzopyran-6-carboxamide was reacted with methanesulfonyl chloride, as described for Example 1 b), to yield (1S)-2-[6-aminocarbonyl)-7-fluoro-3,4-dihydro-1H-2-benzopyran-1-yl]ethyl methanesulfonate.

### c) (1S)-7-Fluoro-1-{2-[(2R)-4-(6-fluoro-1-naphthyl)-2-methylpiperazinyl]ethyl}-3,4-dihydro-1H-2-benzopyran-6-carboxamide

(1S)-2-[6-Aminocarbonyl)-7-fluoro-3,4-dihydro-1H-2-benzopyran-1-yl]ethyl methanesulfonate was coupled with (3R)-1-(6-fluoro-1-naphthyl)-3-methylpiperazine, as described for Example 1 c), to yield (1S)-7-fluoro-1-{2-[(2R)-4-(6-fluoro-1-naphthyl)-2-methylpiperazinyl]ethyl}-3, 4-dihydro-1H-2-benzopyran-6-carboxamide as a pale brown solid. M+H=466.

### EXAMPLE 134

### (1S)-5-Chloro-1-{2-[(2R)-4-(6-fluoro-1-naphthyl)-2-methylpiperazinyl]ethyl}-3,4-dihydro-1H-2-benzopyran-6-carboxamide

### a) (1S)-1-(2-{[tert-Butyl(dimethyl)silyl]oxy}ethyl)-3,4-dihydro-1H-2-benzopyran-6-ol

To a stirred solution of {2-[(1S)-6-bromo-3,4-dihydro-1H-2-benzopyran-1-yl]ethoxy}(tert-butyl)dimethylsilane (0.25 g, 0.67 mmol) in dry tetrahydrofuran (2 mL) at −78° C. under nitrogen was added n-butyllithium (1.6M solution in THF) (0.5 mL, 0.8 mmol). After 45 min, trimethylborate (0.23 mL, 2.02 mmol) was added and the reaction allowed to warm to room temperature over 2 h. N-methylmorpholine (0.245 g, 2.02 mmol) was then added and the resulting mixture heated at reflux for 3.5 h. The reaction was diluted with diethyl ether and washed with water. After separation of the phases, the aqueous phase was further extracted with diethyl ether, and the combined organic extracts dried (Na₂SO₄) and evaporated to dryness. The residue was purified by column chromatography on silica gel, eluting with hexane/ethyl acetate (7:3), to yield (1S)-1-(2-{[tert-butyl(dimethyl) silyl]oxy}ethyl)-3,4-dihydro-1H-2-benzopyran-6-ol as a colourless oil.

### b) (1S)-1-(2-{[tert-Butyl(dimethyl)silyl]oxy}ethyl)-5-chloro-3,4-dihydro-1H-2-benzopyran-6-ol

To a stirred solution of (1S)-1-(2-{[tert-butyl(dimethyl)silyl]oxy}ethyl)-3,4-dihydro-1H-2-benzopyran-6-ol (0.26 g, 0.84 mmol) in dry dichloromethane (5 mL) under nitrogen was added potasium tert-butoxide (0.102 g, 0.885 mmol). After 15 min at room temperature, N-chlorosuccinimide (0.118 g, 0.885 mmol) was added and the reaction mixture stirred for an additional 20 h. The reaction was quenched with saturated aqueous solution of ammonium chloride is and the phases separated. The aqueous phase was extracted into dichloromethane and the combined organic extracts dried (Na₂SO₄) and evaporated to dryness. The resulting residue was purified by column chromatography on silica gel, eluting with hexane/ethyl acetate (8:2), to yield (1S)-1-(2-{[tert-butyl(dimethyl)silyl]oxy)ethyl)-5-chloro-3,4-dihydro-1H-2-benzopyran-6-ol as a colourless oil.

### c) (1S)-1-(2-{[tert-Butyl(dimethyl)silyl]oxy}ethyl)-5-chloro-3,4-dihydro-1H-2-benzopyran-6-yl trifluoromethanesulfonate

To a stirred solution of (1S)-1-(2-{[tert-butyl(dimethyl)silyl]oxy)ethyl)-5-chloro-3,4-dihydro-1H-2-benzopyran-6-ol (0.215 g, 0.627 mmol) in dry tetrahydrofuran (5 mL) under nitrogen was added sodium tert-butoxide (79 mg, 0.69 mmol). After 15 min at room temperature, N-phenyltrifluoromethanesulfonimide (0.251 g, 0.689 mmol) was added and the reaction mixture stirred for an additional 20 h. The reaction was quenched by addition of water and the phases separated. The aqueous phase was extracted with ethyl acetate and the combined organic extracts dried (Na₂SO₄) and concentrated to dryness. The resulting crude product was purified by column chromatography on silica gel, eluting with hexane/ethyl acetate (8:2), to yield (1S)-1-(2-{[tert-butyl(dimethyl)silyl]oxy)ethyl)-5-chloro-3,4-dihydro-1H-2-benzopyran-6-yl trifluoromethanesulfonate as a colourless oil.

### d) (1S)-1-(2-{[tert-Butyl(dimethyl)silyl]oxy}ethyl)-5-chloro-3,4-dihydro-1H-2-benzopyran-6-carbonitrile

To a degassed solution of (1S)-1-(2-{[tert-butyl(dimethyl)silyl]oxy}ethyl)-5-chloro-3,4-dihydro-1H-2-benzopyran-6-yl trifluoromethanesulfonate (0.17 g, 0.358 mmol) in dry dimethylformamide (1 mL) under nitrogen was added zinc (II) cyanide (86 mg, 0.716 mmol) and tetrakistriphenylphosphine palladium (0) (42 mg, 0.036 mmol), and the mixture heated at 120° C. for 1h. The solvent was evaporated in vacuo and the resulting residue purified by column chromatography on silica gel, eluting with hexane/ethyl acetate (9:1), to yield (1S)-1-(2-{[tert-butyl(dimethyl)silyl]oxy}ethyl)-5-chloro-3,4-dihydro-1H-2-benzopyran-6-carbonitrile as a colourless oil.

### e) (1S)-5-Chloro-1-(2-hydroxyethyl)-3,4-dihydro-1H-2-benzopyran-6-carboxamide

To a solution of (1S)-1-(2-{[tert-butyl(dimethyl)silyl]oxy}ethyl)-5-chloro-3,4-dihydro-1H-2-benzopyran-6-carbonitrile (85 mg, 0.241 mmol) in dichloromethane (1 mL) was added 2N sodium hydroxide (0.24 mL, 0.482 mmol), hydrogen peroxide (33% aqueous solution) (1.25 mL, 1.2 mmol) and tetrabutylammonium hydrogen sulfate (21 mg, 0.06 mmol). The mixture was sonicated for 12 h (1 h intervals with breaks of 20 min), then quenched with saturated aqueous potassium hydrogen sulfate (1 mL). Dichloromethane and water were added, the phases separated and the organic phase washed with 10% aqueous solution of sodium thiosulfate, dried (Na₂SO₄) and evaporated to dryness. The resultant solid was dissolved in dry tetrahydrofuran (2 mL) and tetrabutylammonium fluoride (1M solution in tetrahydrofuran) (0.24 mL, 0.24 mmol) added. The mixture was stirred at room temperature for 2 h, then the solvent was removed in vacuo and the residue purified by column chromatography on silica gel, eluting with dichloromethane/methanol (9:1), to yield (1S)-5-chloro-1-(2-hydroxyethyl)-3,4-dihydro-1H-2-benzopyran-6-carboxamide as a white solid.

### f) (1S)-2-[6-Aminocarbonyl)-5-chloro-3,4-dihydro-1H-2-benzopyran-1-yl]ethyl methanesulfonate

(1S)-1-(2-Hydroxyethyl)-5-chloro-3,4-dihydro-1H-2-benzopyran-6-carboxamide was reacted with methanesulfonyl chloride, as described for Example 1 b), to yield (1S)-2-[6-aminocarbonyl)-5-chloro-3,4-dihydro-1H-2-benzopyran-1-yl]ethyl methanesulfonate.

### g) (1S)-5-Chloro-1-{2-[(2R)-4-(6-fluoro-1-naphthyl)-2-methylpiperazinyl]ethyl}-3,4-dihydro-1H-2-benzopyran-6-carboxamide

(1S)-2-[6-Aminocarbonyl)-5-chloro-3,4-dihydro-1H-2-benzopyran-1-yl]ethyl methanesulfonate was coupled with (3R)-1-(6-fluoro-1-naphthyl)-3-methylpiperazine, as described for Example 1 c), to yield (1S)-5-chloro-1-{2-[(2R)-4-(6-fluoro-1-naphthyl)-2-methylpiperazinyl]ethyl}-3,4-dihydro-1H-2-benzopyran-6-carboxamide as a white solid. M+H=482.

### EXAMPLE 135

### 1-{2-[(2R)-4-(6-Cyano-1-naphthyl)-2-methylpiperazinyl]ethyl}-1,3-dihydro-2-benzofuran-5-carboxamide

### a) 5-Chloro-1,3-dihydro-2-benzofuran-1-ol

To a solution of 5-chlorophthalide (3.64 g, 21.6 mmol) in dichloromethane (100 mL) at −78° C. was added di-isobutylaluminium hydride (1M in toluene) (23.8 mL, 23.8 mmol) dropwise. After 1 h the reaction mixture was quenched with a saturated solution of sodium tartrate (250 mL), allowed to warm to room temperature and stirred for 1h. The layers were separated and the aqueous layer extracted with dichloromethane. The combined organic layers were dried (MgSO₄), filtered and concentrated in vacuo to yield the title compound as a white solid.

### b) Ethyl (5-chloro-1,3-dihydro-2-benzofuran-1-yl)acetate

5-Chloro-1,3-dihydro-2-benzofuran-1-ol (2.95 g, 17.3 mmol) was dissolved in THF (60 mL) and cooled to 0° C. Triethyl phosphonoacetate (11.7 g, 52.1 mmol) and cesium carbonate (17 g, 52.1 mmol) were added. After 20 min the cold bath was removed and the reaction mixture allowed to stir at room temperature for 3 h, then quenched with a saturated solution of ammonium chloride and extracted with ethyl acetate. The combined organic layers were washed with brine, dried (MgSO₄), filtered and evaporated in vacuo. Purification by column chromatography, eluting with ethyl acetate/hexane (1:4), yielded the title compound as a colourless oil.

### c) 2-(5-Chloro-1,3-dihydro-2-benzofuran-1-yl)ethanol

Ethyl (5-chloro-1,3-dihydro-2-benzofuran-1-yl)acetate (2.79 g, 11.6 mmol) in THF (60 mL) was cooled to −78° C. and di-isobutylaluminium hydride (1M in toluene) (12.7 mL, 12.7 mmol) was added dropwise. After 1 h, the reaction mixture was quenched with a saturated solution of sodium tartrate (150 mL), allowed to warm to room temperature and stirred for 1 h. The layers were separated and the aqueous layer extracted with ethyl acetate. The combined organic layers were dried (MgSO₄), filtered and evaporated in vacuo. The resultant crude intermediate was dissolved in methanol (50 mL), cooled to 0° C., and sodium borohydride (0.48 g, 12.7 mmol) added in portions. The reaction was quenched with saturated sodium hydrogen carbonate and extracted with ethyl acetate. The combined organic layers were washed with brine, dried (MgSO₄), filtered and evaporated in vacuo. The crude product was purified by column chromatography, eluting with ethyl acetate/hexane (1:2), to yield the title compound as a white solid.

### d) tert-Butyl-[2-(5-chloro-1,3-dihydro-2-benzofuran-1-yl)ethoxy]dimethylsilane

Prepared from 2-(5-chloro-1,3-dihydro-2-benzofuran-1-yl)ethanol, as described in Example 77 a), to yield tert-butyl-[2-(5-chloro-1,3-dihydro-2-benzofuran-1-yl)ethoxy]dimethylsilane.

### e) 1-[2-(tert-Butyldimethylsilanoxy)-ethyl]-1,3-dihydro-2-benzofuran-5-carboxamide

A solution of tert-butyl-[2-(5-chloro-1,3-dihydro-2-benzofuran-1-yl)-ethoxy]-dimethylsilane (0.94 g, 1.48 mmol) in dioxan (2 mL) and a solution of tri-tert-butylphosphine (89 mg, 0.44 mmol) in dioxan (0.7 mL) were added dropwise to a round bottom flask charged with tris (dibenzylideneacetone) dipalladium (136 mg, 0.148 mmol) and zinc cyanide (0.208 g, 1.78 mmol). The flask was fitted with a reflux condenser and the resulting red-purple suspension heated at 120° C. under nitrogen. After 16 h the reaction mixture was cooled to room temperature and diluted with ethyl acetate, filtered through a pad of celite and concentrated in vacuo. The crude product was purified by chromatography on silica, eluting with hexane/ethyl acetate (10:1), to yield the title compound contaminated with dibenzylideneacetone. This crude mixture was dissolved in dichloromethane (3 mL), and tetrabutylammonium hydrogen sulfate (0.29 mmol) was added in one portion. Hydrogen peroxide (30% w/v aqueous solution) (5.75 mmol) and sodium hydroxide (2N aqueous solution) (1.15 mL, 2.3 mmol) were added dropwise The resulting reaction mixture was sonicated for 1 h, then quenched with potassium hydrogen sulfate (4 mL), diluted with dichloromethane, and the layers separated. The organic layer was washed with an aqueous solution of sodium sulfite, dried (MgSO₄), filtered and evaporated in vacuo. The crude product was purified by chromatography on silica, eluting with ethyl acetate/hexane (1:1), to yield the title compound as a white solid.

### f) 1-(2-Hydroxyethyl)-1,3-dihydro-2-benzofuran-5-carboxamide

Prepared from 1-[2-(tert-butyldimethylsilanoxy)ethyl]-1,3-dihydro-2-benzofuran-5-carboxamide, as described in Example 1 a).

### g) 1-(2-[(2R)-4-(6-Cyano-1-naphthyl)-2-methylpiperazinyl]ethyl}-1,3-dihydro-2-benzofuran-5-carboxamide

The title compound was prepared from 1-(2-hydroxyethyl)-1,3-dihydro-2-benzofuran-5-carboxamide by initial formation of the methanesulfonate as described for Example 1 b), and condensation of this sulfonate with (3R)-5-(3-methylpiperazin-1-yl)naphthalene-2-carbonitrile, as described for Example 58 d). The mixture of diastereomers was separated by chiral HPLC using a Chiracel OJ column, eluting with hexane/ethanol (1:1) with 0.2% dimethylethylamine. M+H=441.

The following Examples illustrate typical formulations containing the compound of the invention.

### EXAMPLE 136

Tablets each containing 10 mg of active ingredient are made up as follows:

| | |
|---|---|
| Active ingredient | 10 mg |
| Starch | 160 mg |
| Microcrystalline cellulose | 100 mg |
| Polyvinylpyrrolidone (as 10% solution | 13 mg |
| in water) | |
| Sodium carboxymethyl starch | 14 mg |
| Magnesium stearate | 3 mg |
| Total | 300 mg |

The active ingredient, starch and cellulose are mixed thoroughly. The solution of polyvinylpyrrolidone is mixed with the resultant powders and passed through a sieve. The granules so produced are dried and re-passed through a sieve. The sodium carboxymethyl starch and magnesium stearate are then added to the granules which, after mixing, are compressed on a tablet machine to yield tablets each weighing 300 mg.

### EXAMPLE 137

Capsules each containing 20 mg of medicament are made as follows:

| | |
|---|---|
| Active ingredient | 20 mg |
| Dried starch | 178 mg |
| Magnesium stearate | 2 mg |
| Total | 200 mg |

The active ingredient, starch and magnesium stearate are passed through a sieve and filled into hard gelatine capsules in 200 mg quantities.

1. A compound of formula (I) 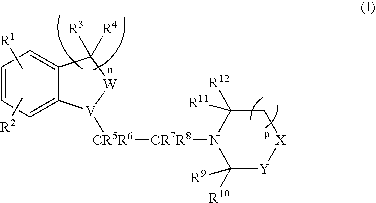
in which
R¹ is 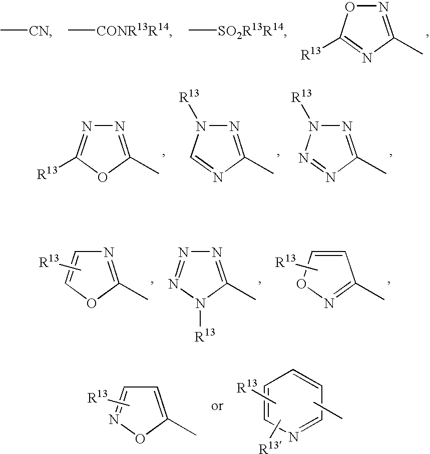
where R¹³ and R¹⁴ are each hydrogen or C₁₋₆ alkyl, or R¹³ and R¹⁴ taken together with the nitrogen atom to which they are attached form a morpholino, pyrrolidino or piperidinyl ring optionally substituted with one or two C₁₋₆ alkyl groups;
R¹³′ is selected from hydrogen, C₁₋₆ alkyl, C₁₋₆ alkoxy, carboxy, hydroxy, cyano, halo, trifluoromethyl, nitro, amino, C₁₋₆ acylamino, C₁₋₆ alkylthio, phenyl or phenoxy;
R² is one of the values defined for R¹, or hydrogen, C₁₋₆ alkyl, C₁₋₆ alkoxy or halo;
R³, R⁴, R⁵, R⁶, R⁷, R⁸, R¹⁰ and R¹² are each hydrogen or C₁₋₆ alkyl;
R⁹ and R¹¹ are each hydrogen, C₁₋₆ alkyl or —(CH₂)_{q}—OR²⁰, wherein R²⁰ is C₁₋₆ alkyl;
n is 1 or 2;
p is 0, 1 or 2;
q is 1 or 2; 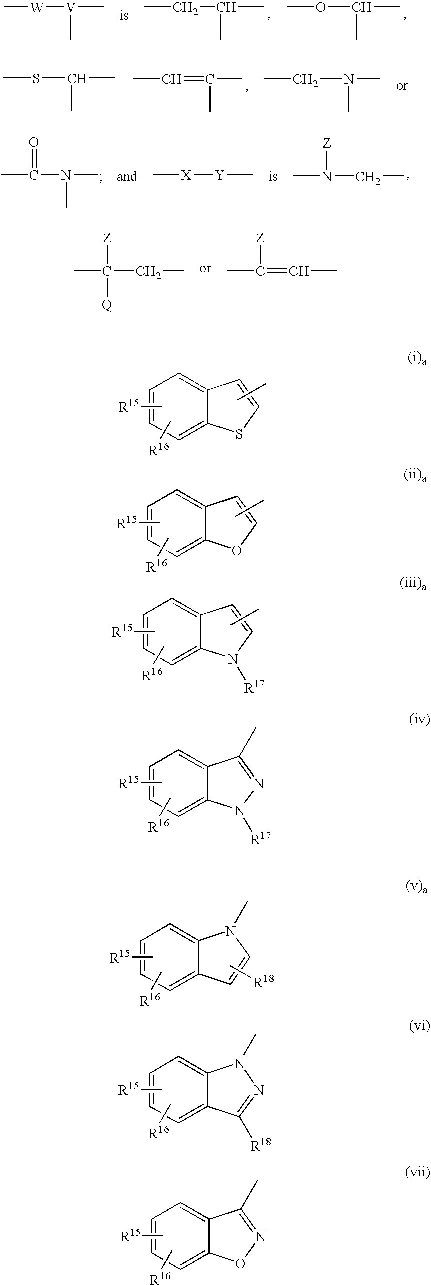 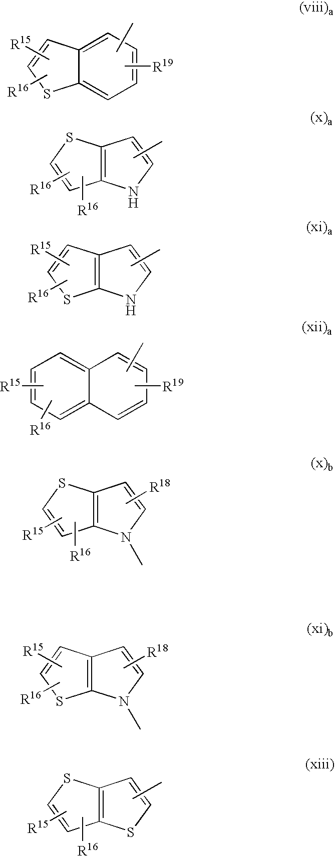
where R¹⁵, R¹⁶ and R¹⁹ are each hydrogen, halo, C₁₋₆ alkyl or C₁₋₆ alkoxy, carboxy-C₁₋₆ alkyl, cyano, halogen, trifluoromethyl, trifluoromethoxy, nitro, amino, C₁-C₆ acylamino and C₁-C₆ alkylthio; and R¹⁷ and R¹⁸ are each hydrogen or C₁₋₆ alkyl;
Q is hydrogen, halo, nitrile, carboxy-C₁₋₆ alkyl, hydroxy, C₁₋₆ alkyl or C₁₋₆ alkoxy;
and pharmaceutically acceptable salts thereof.

2. A compound according to claim 1 wherein R² is hydrogen, C₁₋₆ alkyl, C₁₋₆ alkoxy or halo.

3. A compound according to claims 1 or 2 wherein

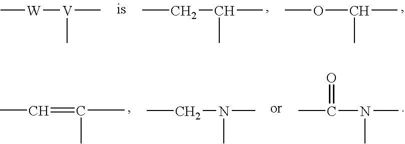

4. A compound according to any one of claims 1 to 3 wherein groups R³ to R¹² are hydrogen, or R³ to R¹⁰ and R¹² are hydrogen and R¹¹ s C₁₋₆ alkyl.

5. A compound according to any one of claims 1 to 4 wherein R¹ is —CONR¹³R¹⁴, and R¹³ and R¹⁴ are each hydrogen or methyl.

6. A compound according to any one of claims 1 to 5 wherein R² is hydrogen.

7. A compound according to any one of claims 1 to 6 wherein R² is hydrogen, chloro, fluoro or methyl.

8. A compound according to any one of claims 1 to 7 wherein p is 0 or 1.

9. A compound according to any one of claims 1 to 8 wherein R¹⁵, R¹⁶ and R¹⁸ are each hydrogen, halo, cyano or methoxy.

10. A compound according to any one of claims 1 to 9 wherein R¹⁷ is hydrogen or C₁₋₆ alkyl.

11. A compound according to any one of claims 1 to 10 wherein R¹⁹ is hydrogen, halo, cyano or C₁₋₆ alkyl.

12. A compound according to any one of claims 1 to 11 wherein R¹¹ is methyl, ethyl or propyl.

13. A compound according to any one of claims 1 to 11 wherein R¹¹ is C₁₋₆ alkyl or —(CH₂)_{q}—OR²⁰, and R²⁰ is C₁₋₆ alkyl.

14. A compound according to claim 13 wherein q is 1.

15. A compound according to any one of claims 1 to 14 wherein Z is

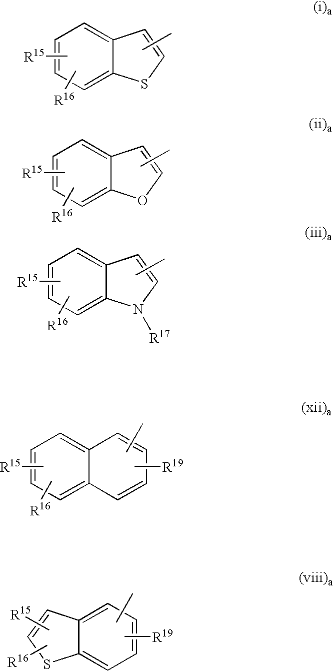

16. A compound according to any one of claims 1 to 15 wherein Z is

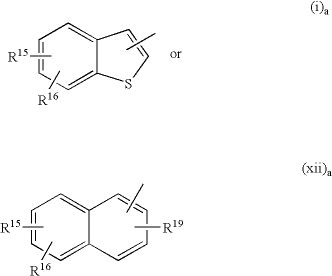

17. A compound of the formula II 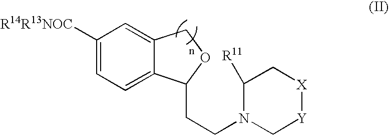
in which n is 1 or 2, R¹³ and R¹⁴ are each hydrogen or C₁₋₆ alkyl, and are preferably both hydrogen, R¹¹ is hydrogen or C₁₋₆ alkyl, preferably methyl, and —X—Y— is 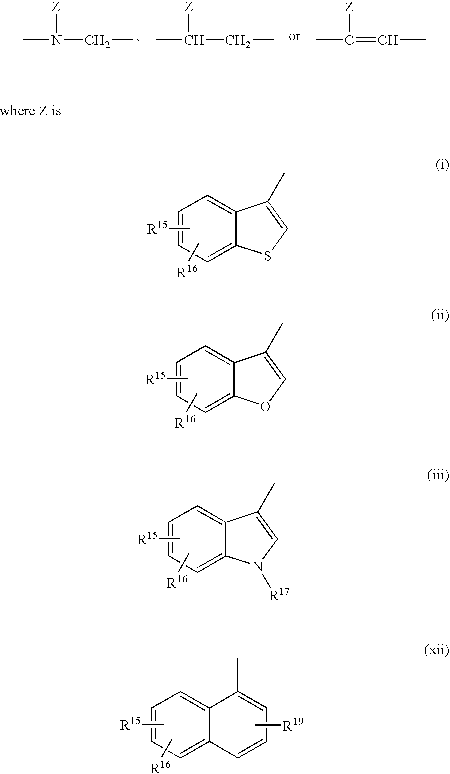
and R¹⁵, R¹⁶ and R¹⁹ are each hydrogen, halo or alkoxy, and R¹⁷ is hydrogen or C₁₋₆ alkyl; and pharmaceutically acceptable salts thereof.

18. A compound of the formula: 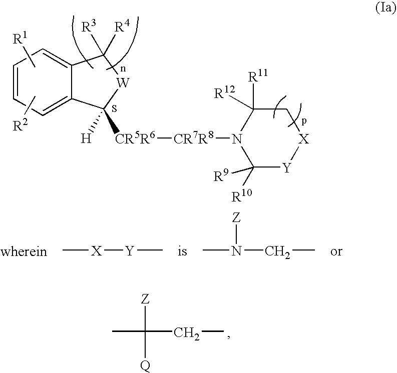
R¹ to R¹², Q, Z, n and p have the values defined for formula I as claimed in any one of claims 1 to 16, —W— is —CH₂—, —O—, or —S—.

19. A compound of formula: 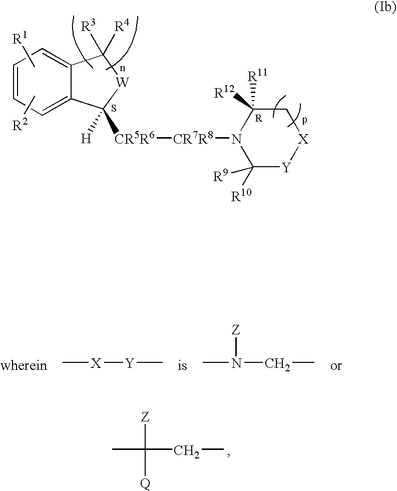
R¹ to R¹², Q, Z, n and p have the values defined for formula I as claimed in any one of claims 1 to 16, —W— is —CH₂—, —O—, or —S—.

20. A compound of formula: 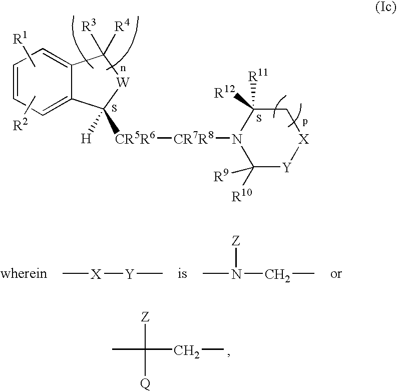
R¹ to R¹², Q, Z, n and p have the values defined for formula I as claimed in any one of claims 1 to 16, —W— is —CH₂—, —O—, or —S—.

21. A compound of formula: 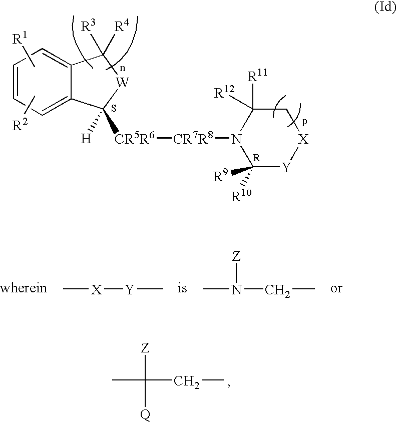
R¹ to R¹², Q, Z, n and p have the values defined for formula I as claimed in any one of claims 1 to 16, —W— is —CH₂—, —O—, or —S—.

22. A compound of formula: 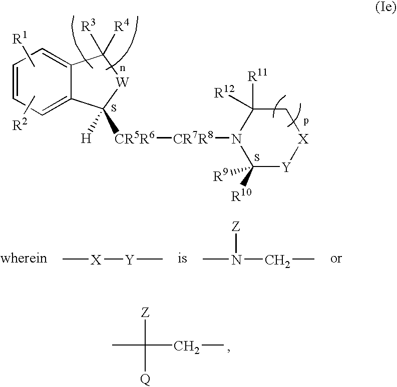
R^{1 to R}¹², Q, Z, n and p have the values defined for formula I as claimed in any one of claims 1 to 16, —W— is —CH₂—, —O—, or —S—.

23. A pharmaceutical formulation comprising a compound of formula I as claimed in any one of claims 1 to 22, or a pharmaceutically acceptable salt thereof, together with a pharmaceutically acceptable carrier, diluent or excipient.

24. A process for preparing a compound of formula I as claimed in any one of claims 1 to 16, or a salt or ester thereof, which comprises reacting a compound having the formula: 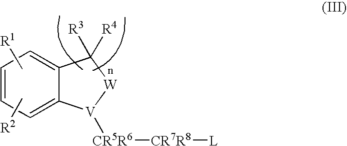
where L is a leaving group, with a compound of the formula: 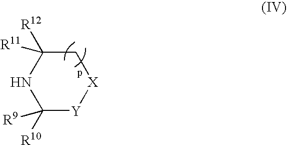
where R¹ to R¹², —W—V—, —X—Y—, m and n have the values defined in claim 1.

25. A compound of formula I as claimed in any one of claims 1 to 22, or a pharmaceutically acceptable salt thereof, for use as a pharmaceutical.

26. A compound of formula I as claimed in any one of claims 1 to 22, or a pharmaceutically acceptable salt thereof, for use in treating a disorder of the central nervous system.

27. Use of a compound of formula I as claimed in any one of claims 1 to 22, or a pharmaceutically acceptable ester thereof, or a pharmaceutically acceptable salt thereof, for the manufacture of a medicament for treating a disorder of the central nervous system.

28. A method of treating an animal, including a human, suffering from or susceptible to a disorder of the central nervous system, which comprises administering a compound according to claim 1, or a pharmaceutically acceptable salt or ester thereof.
